(19) United States
(12) Patent Application Publication
(10) Pub. No.: US 2007/0178148 A1
(43) Pub. Date: Aug. 2, 2007

(54) Preparations of growth hormone

(75) Inventors: Vidadi Yusibov, Havertown, PA (US); Shailaja Rabindran, Newark, DE (US); Marina Skarjinskaia, Newark, DE (US); Oleg Fedorkin, Newark, DE (US); Burt Ensley, Sedona, AZ (US)

Correspondence Address: CHOATE, HALL & STEWART LLP, TWO INTERNATIONAL PLACE, BOSTON, MA, 02110, US

(73) Assignee: Fraunhofer U.S.A. Inc.

(21) Appl. No.: 11/347,872

(22) Filed: Feb. 5, 2006

Related U.S. Application Data
(63) Continuation-in-part of application No. 11/061,980, filed on Feb. 18, 2005.
(63) Continuation-in-part of application No. 10/294,314, filed on Nov. 12, 2002.
(63) Continuation-in-part of application No. 10/770,600, filed on Feb. 3, 2004.
(60) Provisional application No. 60/650,066, filed on Feb. 4, 2005.
(60) Provisional application No. 60/546,339, filed on Feb. 20, 2004.
(60) Provisional application No. 60/444,615, filed on Feb. 3, 2003.

(51) Int. Cl. A61K 38/27; A61K 9/48; A61K 9/20

(52) U.S. Cl. 424/451; 514/12; 424/464

(57) ABSTRACT

The present invention relates to compositions and systems for expressing pharmaceutically active gene products in plants. In particular, the invention provides compositions, systems and methods for the production of human growth hormone in plants. Provided are nucleic acid and protein sequences, expression and vector constructs, host cells and plants capable of expressing human growth hormone, and compositions and kits comprising produced human growth hormone. Additionally provided are methods for production and use of the compositions of the invention. Therapeutic use of the produced human growth hormone is also provided

### CROSS REFERENCE TO RELATED APPLICATIONS

This application claims the benefit of U.S. Provisional Patent Application No. 60/650,066 filed Feb. 4, 2005; this application is a continuation-in-part application of U.S. patent application Ser. No. 11/061,980 filed Feb. 18, 2005, which claims the benefit of U.S. Provisional Patent Application No. 60/546,339 filed Feb. 20, 2004; this application is a continuation-in-part application of U.S. patent application Ser. No. 10/770,600 filed Feb. 3, 2004 which claims the benefit of U.S. Provisional Patent Application No. 60/444,615 filed Feb. 3, 2003; and this application is a continuation-in-part application of U.S. patent application Ser. No. 10/294,314 filed Nov. 12, 2002. Each of the foregoing applications is hereby incorporated herein by reference.

### BACKGROUND OF THE INVENTION

Biotherapeutic proteins and peptides for preventative and therapeutic use are of paramount importance to health and medicine. However, several factors contribute to the high costs of producing pharmaceuticals, and result in the high price of the pharmaceuticals for the consumer. This is particularly true for protein and peptide-based medications. Another contributing factor for some medications is the inability to administer therapeutically effective amounts of the pharmaceutical agent orally. For example, FDA approved protein and peptide pharmaceuticals, such as human growth hormone and insulin, can currently only be administered by injection.

Historically, many pharmaceutical proteins and peptides have been recovered from human or animal sources. More recently, a variety of heterologous expression systems have been developed. Bacterial expression systems are relatively easy to manipulate and the yield of the product is high. However, mammalian proteins often require extensive posttranslational modification for functional activity, which can be a limiting factor in bacterial expression systems. Cell culture systems such as mammalian, human, and insect cell culture systems are often more appropriate for the production of complex proteins, but long lead times, low recovery of the product, possible pathogen transfer, and high capital and production costs present serious concerns. Transgenic animals have been employed for production of heterologous proteins. Unfortunately, this system is limited by the long period of time it takes to generate new and improved products and the risks of pathogen transfer to human subjects. Thus, low quantities of active target product in the source material coupled with immense production costs, and more importantly, safety, have limited the availability of biotherapeutics and vaccines for prevention and treatment of many diseases around the world.

The economic and biochemical limitations to producing pharmaceutical proteins and peptides in prokaryotic and eukaryotic cells have led researchers to examine plants as new hosts for large-scale production of proteins and peptides with the expectation of reduced cost. Although plants are less expensive to grow and harvest in bulk than other prokaryotic and eukaryotic cells, limitations in plant systems remain. For example, expression initially is often typically low, and/or harvesting of material can result in degradation of the transgenically expressed protein even before purification of the protein or direct consumption of the plant containing therapeutic protein. Viral vector systems have proven to be particularly useful, but viruses may infect non-target plants, potentially posing significant environmental risks. Also, many available engineered plant viruses do not express inserted genes at desired levels, and/or in desired target plants or tissues. Furthermore, virus stability can be problematic. Thus, there remains a need for developing improved systems for expressing transgenes in plants, including systems that would allow for greater flexibility and control.

### SUMMARY OF THE INVENTION

The present invention provides a system for producing growth hormone (e.g., human growth hormone (hGH)) in plants, and further provides preparations, including oral preparations, containing such growth hormone. Additionally provided are methods and compositions for delivery of a physiologically significant dose of growth hormone or a pharmaceutically active portion thereof. Provided methods and compositions relate generally to effective delivery of growth hormone by any method. In certain aspects the methods and compositions provide for oral or transmucosal delivery of growth hormone or a pharmaceutically active portion thereof. Human growth hormone or growth hormone from another animal may be used. Methods of use of the provided compositions for treatment of growth hormone deficiency disorder are further provided. The methods provided herein for administration of plant-produced growth hormone or a pharmaceutically active portion thereof may utilize known methods in the art for plant production of transgenic proteins. Additionally, the invention provides methods and compositions for production of growth hormone or a pharmaceutically active portion thereof in plants.

Growth hormone polypeptides may be produced in accordance with the present invention in any of a variety of plant expression systems. For example, the present invention provides, among other things, vector systems for viral infection of plants, or portions of plants; root systems, which can be grown into clonal plants; and sprouted seedling systems. The provided systems and methods, or any methods known in the art may be used in accordance with the present invention. In some embodiments, the methods and plants produce and/or utilize transient expression. In other embodiments, the methods and plants produce and/or utilize transgenic expression. In some embodiments the systems are designed to minimize risk of environmental contamination. In certain embodiments, growth hormone polypeptide(s) is/are produced in edible plants or portions thereof.

Provided are methods of treating a subject with a pharmaceutically active protein produced by the production methods described herein. For example, a pharmaceutically active growth hormone or a pharmaceutically active portion thereof is expressed in a plant or portion thereof by growing a plant or portion thereof in a contained, regulatable environment, wherein the plant or portion thereof contains an expression cassette that is capale of inducing expression of growth hormone or a pharmaceutically active portion thereof in the plant; and administering the plant or portion thereof expressing the pharmaceutically active growth hormone to the subject. In one example a pharmaceutically active growth hormone or a pharmaceutically active portion thereof is expressed in a sprout (e.g., sprouted seedling) by growing a sprout in an environment, wherein the sprout contains an expression cassette that includes promoter capable of inducing expression of the pharmaceutically active protein in the sprouted seedling; and administering the sprouted seedling expressing the pharmaceutically active protein, or an extract thereof to the subject. In another example a pharmaceutically active growth hormone or a pharmaceutically active portion thereof is expressed in a clonal entity (e.g., a clonal root, clonal root line, clonal cell, clonal cell line, clonal plant) by growing a clonal entity in an environment, wherein the clonal entity contains an expression cassette that is capable of inducing expression of the pharmaceutically active growth hormone in the clonal entity; and administering the clonal entity expressing the pharmaceutically active protein or an extract thereof to the subject.

### DESCRIPTION OF THE DRAWING

FIG. 1 presents a schematic diagram of the engineering of a TMV based viral construct containing a polynucleotide encoding growth hormone or a pharmaceutically active portion thereof. The upper portion of the figure shows a diagram of the genomic organization of a TMV based virus construct, D4, and the lower portion shows the same construct following insertion of a polynucleotide encoding growth hormone or a pharmaceutically active portion thereof (e.g., a gene encoding hGH, indicated as “target”). The 126/183 kDa protein is required for replication of the virus. The 30 kD protein is the movement protein (MP) that mediates cell-to-cell movement. Arrows indicate positions of the subgenomic promoters. Transcription of the inserted polynucleotide is under control of the TMV CP subgenomic promoter. The 3′ portion of the construct includes TMV coat protein sequences and untranslated regions. These portions are optional.

FIG. 2 presents a schematic diagram of the engineering of a TMV based viral construct containing a polynucleotide encoding growth hormone. The upper portion of the figure shows a schematic diagram of the genomic organization of a TMV based virus construct, 30B. The lower portion shows the same construct following insertion of a polynucleotide encoding growth hormone or a pharmaceutically active portion thereof (e.g., a gene encoding hGH, indicated as “target”). The 126/183 kDa protein is required for replication of the virus. The 30 kD protein is the movement protein (MP) that mediates cell-to-cell movement. CP is the coat protein that mediates systemic spread. Arrows indicate positions of the subgenomic promoters. Transcription of the inserted polynucleotide is under control of an introduced promoter. CP expression is under control of the endogenous CP promoter. The 3′ portion of the construct includes TMV coat protein sequences and untranslated regions. These portions are optional.

FIG. 3 presents a schematic diagram of the engineering of a TMV based viral construct containing a polynucleotide encoding growth hormone or a pharmaceutically active portion thereof and a gene encoding a marker for detection and/or selection. The upper portion of the figure shows the genomic organization of a TMV based virus construct, D4. The middle portion of the figure shows the same construct after insertion of a gene encoding a detectable marker (GFP) replacing the MP coding sequence. The lower portion of the figure shows the same construct following insertion of a polynucleotide encoding growth hormone or a pharmaceutically active portion thereof (e.g., a gene encoding hGH, indicated as “target”). The 126/183 kDa protein is required for replication of the virus. Arrows indicate positions of the subgenomic promoters. Transcription of the detectable marker is under control of the MP subgenomic promoter. Transcription of the inserted polynucleotide encoding growth hormone or a pharmaceutically active portion thereof is under control of the TMV CP subgenomic promoter. The 3′ portion of the construct includes TMV coat protein sequences and untranslated regions. These portions are optional.

FIG. 4 presents a schematic diagram of the engineering of a TMV based viral construct containing a polynucleotide encoding growth hormone or a pharmaceutically active portion thereof and a gene encoding a marker for detection and/or selection. The upper portion of the figure shows the genomic organization of a TMV based virus construct, D4. The middle portion of the figure shows the same construct after insertion of a gene encoding a selectable marker (gene encoding resistance to kanamycin) replacing the MP coding sequence. The lower portion of the figure shows the same construct following insertion of a polynucleotide encoding growth hormone or a pharmaceutically active portion thereof (e.g., a gene encoding hGH, indicated as “target”). The 126/183 kDa protein is required for replication of the virus. Arrows indicate positions of the subgenomic promoters. Transcription of the selectable marker is under control of the TMV MP subgenomic promoter. Transcription of the inserted polynucleotide encoding growth hormone or a pharmaceutically active portion thereof is under control of the TMV CP subgenomic promoter. The 3′ portion of the construct includes TMV coat protein sequences and untranslated regions. These portions are optional.

FIG. 5 presents a schematic diagram of the engineering of AIMV based viral constructs containing a polynucleotide encoding growth hormone or a pharmaceutically active portion thereof either as an independent open reading frame or as a genetic fusion with AIMV CP coding sequences. The upper portion of the figure shows the genomic organization of RNA3 of AIMV, which includes genes encoding CP and MP as well as containing 5′ and 3′ UTRs and a subgenomic promoter. The left side of the figure shows a construct in which transcription of an mRNA containing separate open reading frames that encode a polypeptide encoding growth hormone or a pharmaceutically active portion thereof (indicated as “target”) and the AIMV CP is under control of the AIMV subgenomic promoter. The right side of the figure shows a construct in which transcription of an mRNA containing a single open reading frame containing a polynucleotide encoding growth hormone or a pharmaceutically active portion thereof and CP coding sequences is under control of the AIMV CP subgenomic promoter. The open reading frame encodes a fusion protein in which a polypeptide encoding growth hormone or a pharmaceutically active portion thereof is fused to CP.

FIG. 6 shows a Western blot analysis to screen clonal root lines each derived from individual plant cells that were infected with a viral vector whose genome contains a gene that encodes human growth hormone (hGH) under control of the TMV CP promoter. Root lines were screened 30 days after separation of the root from the leaf from which it was derived. Root lines demonstrating high levels of expression are indicated with arrows. C— represents control lanes containing no protein. MWM represents molecular weight markers. hGH represents recombinant human growth hormone.

FIG. 7 shows a Western blot analysis demonstrating hGH production in selected clonal root lines derived from plant cells into which a viral vector whose genome contains a gene that encodes hGH under control of the TMV CP promoter was introduced. The analysis was performed following 10 subculturings after separation of the roots from the leaves from which they were derived. C— represents a control lane containing no protein. MWM represents molecular weight markers. hGH represents recombinant human growth hormone.

FIG. 8A shows a clonal plant that was obtained from a clonal root line derived from a plant cell into which a viral vector encoding hGH was introduced. FIG. 8B shows lesion formation in a sensitive host plant that was inoculated with a small leaf sample from the clonal plant, indicating that the clonal plant regenerated from the clonal root line maintains active viral replication. To test if the plant maintains virus replication a small leaf sample was used to inoculate a tobacco variety that is a host for formation of local lesions. Formation of lesions within 2 days of inoculation (see arrows) indicates that the clonal plant line regenerated from a clonal root line maintains active virus replication.

FIG. 9 presents a schematic representation of certain families of viruses that infect plants.

FIG. 10 shows representative examples of tobamovirus genomes.

FIG. 11 is a schematic representation of different strategies for foreign gene expression using plant virus-based vectors.

FIG. 12 is a schematic representation of AIMV and TMV genomes.

FIG. 13 is a picture of a Western blot of human growth hormone (hGH) production in N. benthamiana plants infected with in vitro transcripts of GH.

FIG. 14 is a schematic representation of transformation constructs for expression of recombinant proteins in Brassica juncea.

FIG. 15 is a picture of an immunoblot of transgenic Brassica juncea expressing human growth hormone under control of the HSP18.2 promoter.

FIG. 16 gives a representative list of accession codes for various TMV genome sequences.

FIG. 17 presents accession codes for a variety of AIMV genome sequences.

FIG. 18 presents a schematic diagram of the genomic organization of 125C (FIG. 18A) and D4 following insertion of a polynucleotide of interest (FIG. 18B). The 126/183 kDa protein is required for replication of the virus. The MP is the movement protein that mediates cell-to-cell movement. Arrows indicate positions of the subgenomic promoter. The shaded region represents TMV coat protein sequences that contain a cis element that may be required for optimal replication. The black box represents a polynucleotide of interest, e.g., a foreign gene.

FIG. 19 shows a Western blot of protoplasts infected with in vitro synthesized transcripts of 125C/hGH. Samples were analyzed 24 hours post inoculation. 1 ug of purified hGH was loaded as a standard.

FIG. 20 is a Western blot showing detection of hGH in N. benthamiana plants 11 days post infection (dpi).

FIGS. 21a-21d presents schematics of various D4-related vectors. 126/183 kDa are the replicase proteins, MP is the movement protein required for cell-to-cell movement. Nucleotide numbers represent positions in the wild type TMV genome. C3GFP is the cycle3 mutant of green fluorescent protein (GFP) (Crameri A, Whitehorn E A, Tate E, Stemmer W P, Nat. Biotechnol., 14(3): 315-9, 1996). The asterisk indicates mutated C3GFP in which the NcoI site and the XhoI sites in the ORF have been eliminated by mutation using PCR. PstI-XhoI sites were used to introduce sequences from AIMV RNA3 that include the origin of assembly (OAS).

FIG. 22 depicts results of weight gain as an effect of hGH administration in rats.

FIG. 23. depicts results of weight gain as an effect of oral or subcutaneous administration of hGH in rats. Each colored bar represents a different animal. PBS=saline control (gavage); Com hGH or =commercial hGH (Lilly) administered orally as enterically coated tablets (250 micrograms per animal per day—single daily dose); P1 hGH or =plant-produced hGH, extracted and lyophilized; lyophilized material was formulated as enterically coated tablets (250 micrograms per day per animal—single daily dose); PBS par=saline control subcutaneous injection; Corn hGH par=commercial hGH (Lilly) administered subcutaneously (60 micrograms per day per animal—single daily dose); P1 hGH par=plant-produced hGH, extracted and lyophilized and administered as 60 micrograms per animal per dose per day by subcutaneous injection.

### DEFINITIONS

“Administration” of a pharmaceutically active peptide or protein or a therapeutically active peptide or protein to a subject in need thereof is intended as providing the pharmaceutically active protein to such subject in a manner that retains the therapeutic effectiveness of such protein for a length of time sufficient to provide a desired beneficial effect to such subject. “Oral administration” of a pharmaceutically active peptide or protein means primarily administration by way of the mouth, by eating or ingesting, but also intends to include any administration that provides such peptides or proteins to the subject's stomach or digestive track. Where oral administration is utilized, administration results in contact of the pharmaceutically active protein with the gut mucosa.

Approximately. “Approximately” in reference to a number includes numbers that fall within a range of 5% in either direction (greater than or less than) the number unless otherwise stated or otherwise evident from the context (except where such number would exceed 100% of a possible value). Where ranges are stated, the endpoints are included within the range unless otherwise stated or otherwise evident from the context.

Clonal. For the purpose of the present invention, the term clonal as applied, e.g., to a plant or plant tissue such as a root, leaf, stem, etc., means that the plant or plant tissue was derived from a single ancestral cell. In general, the cells or a clonal plant or plant tissue will be genetically identical with the exception of somatic mutations or other genetic alterations that may arise in descendant cells (e.g., through either natural or artificial introduction of a new gene into a descendant cell, telomere shortening, etc.). Typically the genome of the cells will be at least 95% identical, at least 98% identical, at least 99% identical, at least 99.5% identical, at least 99.9% identical.

“Expression” refers to transcription and/or translation of an endogenous gene or a transgene in plants. “Expression cassette” or “expression vector” refers to a nucleic acid sequence (e.g., DNA sequence, RNA sequence) capable of directing expression of a particular nucleotide sequence in an appropriate host cell. An expression cassette typically includes a promoter operably linked to the nucleotide sequence encoding growth hormone or a pharmaceutically active portion thereof, which is optionally operably linked to 3′ sequences, such as 3′ regulatory sequences or termination signals. It also typically includes sequences required for proper translation of the nucleotide sequence. An expression vector typically comprises an expression cassette and further comprises further sequences which are utilized for maintenance of the expression cassette in a host cell. According to the present invention, the coding region of an expression cassette or an expression vector usually codes for a growth hormone protein or a pharmaceutically active portion thereof, but may also code for a functional RNA of interest, for example. The expression cassette including the nucleotide sequence of interest may be chimeric, meaning that the nucleotide sequence includes more than one nucleic acid sequence of distinct origin that are fused together by recombinant DNA techniques, resulting in a nucleotide sequence that does not occur naturally and that particularly does not occur in the plant to be transformed. The expression cassette may also be one that is naturally occurring but has been obtained in a recombination form useful for heterologous expression. Often, however, the expression cassette is heterologous with respect to the host, i.e., the particular DNA sequence of the expression cassette does not occur naturally in the host cell and must have been introduced into the host cell or an ancestor of the host cell by a transformation event. The expression of the nucleotide sequence in the expression cassette may be under the control of a constitutive promoter or of an inducible promoter that initiates transcription only when the host cell is exposed to some particular stimulus. In the case of a multicellular organism, such as a sprouted seedling, the promoter can also be specific to a particular tissue, organ, or stage of development. A nuclear expression cassette is usually inserted into the nuclear genome of a plant and is capable of directing the expression of a particular nucleotide sequence from the nuclear genome of the plant. A plastid expression cassette is usually inserted into the plastid genome of a plant and is capable of directing the expression of a particularly nucleotide sequence from the plastid genome of the plant. In the case of a plastid expression cassette, for expression of nucleotide sequence from a plastid genome, additional elements, i.e., ribosome binding sites, or 3′ stem-loop structures that impede plastid RNA polyadenylation and subsequent degradation may be required.

A “food” or “food product” is a liquid or solid preparation of sprouted seedlings of the invention that can be ingested by humans or other animals. The terms include preparations of the raw or live sprouted seedlings and sprouted seedlings that may be fed directly to humans and other animals. Materials obtained from a sprouted seedling are intended to include a whole edible sprouted seedling that can be ingested by a human or other animal. The term may also include any processed sprouted seedling together with a nutritional carrier that is fed to humans and other animals. Processing steps include steps commonly used in the food or feed industry. Exemplary steps include, but are not limited to concentration or condensation of the solid matter of the sprouted seedling (e.g., to form, for example, a pellet, production of a paste), drying, or lyophilization, cutting, mashing, or grinding of the plant to various extents, or extraction of the liquid part of the plant to produce a soup, a syrup, or a juice. A processing step can also include cooking, e.g., steaming, the sprouted seedlings. A medicinal food includes a composition that is ingested by a subject for an intended therapeutic effect on the subject. A medical food may be ingested alone or may be administered in combination with a pharmaceutical composition known in the art. A medical food includes the equivalent feedstuff for non-human animals.

Gene: For the purposes of the present invention, the term gene has its meaning as understood in the art. In general, a gene is taken to include gene regulatory sequences (e.g., promoters, enhancers, etc.) and/or intron sequences, in addition to coding sequences (open reading frames). It will further be appreciated that the definition of gene can include nucleic acids that do not encode proteins but rather provide templates for transcription of functional RNA molecules such as tRNAs, rRNAs, microRNAs (miRNAs), short hairpin RNAs (shRNAs), short interfering RNAs, (siRNAs), etc. For the purpose of clarity we note that, as used in the present application, the term “gene” generally refers to a nucleic acid that includes a portion that encodes a protein; the term may optionally encompass regulatory sequences such as promoters, enhancers, terminators, etc. This definition is not intended to exclude application of the term “gene” to non-protein coding expression units but rather to clarify that, in most cases, the term as used in this document refers to a protein coding nucleic acid.

Gene product or expression product: A gene product or expression product is, in general, an RNA transcribed from a gene or polynucleotide, or a polypeptide encoded by an RNA transcribed from the gene or polynucleotide. Expression of a gene or a polynucleotide refers to (i) transcription of RNA from the gene or polynucleotide; (ii) translation of RNA transcribed from the gene or polynucleotide, or both (i) and (ii). Other steps such as processing, translocation, etc., may also take place in the course of expression or thereafter.

“Growth hormone:” As used herein, “growth hormone” is intended to encompass biologically active growth hormone protein, or a pharmaceutically active portion thereof, which is produced in a plant or portion thereof. Growth hormone proteins may be naturally-occurring growth hormone proteins, or may be designed or engineered proteins. The sequence of biologically active growth hormone of various species are well known in the art and may be utilized and adapted accordingly for use in the present invention. For example, human growth hormone (SWISSPROT accession no: P01241) is well known and accessible in the art, and has been characterized to identify functional fragments and variants which retain biological activity of naturally active human growth hormone. For example, encoded protein may be full length growth hormone (e.g., human growth hormone) which consists of the naturally occurring growth hormone protein sequence. Growth hormone proteins also may be a protein fragment of full length growth hormone which retains functional activity of full length growth hormone. Furthermore, growth hormone proteins of use in the present invention include a modified amino acid sequence of full length growth hormone, which is at least 85%, at least 90%, at least 95%, at least 99% or more identical to the naturally occurring growth hormone protein sequence, and wherein the variant protein retains functional activity of full length, pharmaceutically active growth hormone

“Heterologous sequences,” as used herein, means of different natural origin or of synthetic origin. For example, if a host cell is transformed with a nucleic acid sequence that does not occur in the untransformed host cell, that nucleic acid sequence is said to be heterologous with respect to the host cell. The transforming nucleic acid may include a heterologous promoter, heterologous coding sequence, or heterologous termination sequence. Alternatively, the transforming nucleic acid may be completely heterologous or may include any possible combination of heterologous and endogenous nucleic acid sequences. Similarly, heterologous refers to a nucleotide sequence derived from and inserted into the same natural, original cell type, but which is present in a non-natural state, e.g., a different copy number, or under the control of different regulatory elements.

The term “inducible promoter,” means a promoter that is activated by the presence or absence of a particular stimulus that increases promoter activity directly or indirectly. Some non-limiting examples of such stimuli include heat, light, developmental regulatory factors, wounding, hormones, and chemicals, e.g., small molecules. One example of a light-inducible promoter is the ribulose-5-phosphate carboxylase promoter. Chemically-inducible promoters also include receptor-mediated systems, e.g., those derived from other organisms, such as steroid-dependent gene expression, the Lac repressor system and the expression system utilizing the USP receptor from Drosophila mediated by juvenile growth hormone and its agonists, described in WO 97/13864, incorporated herein by reference, as well as systems utilizing combinations of receptors, e.g., as described in WO 96/27673, also incorporated herein by reference. Additional chemically inducible promoters include elicitor-induced promoters, safener-induced promoters as well as the alcA/alcR gene activation system that is inducible by certain alcohols and ketones (WO 93/21334; Caddick et al. (1998) Nat. Biotechnol. 16:177-180, the contents of which are incorporated herein by reference). Wond inducible promoters include promoters for proteinase inhibitors, e.g., proteinase inhibitor 11 promoter from potato, and other plant-derived promoters involved in the wound response pathway, such as promoters for polyphenyl oxidases, LAP, and TD. See, e.g., Gatz “Chemical Control of Gene Expression,” Ann. Rev. Plant Physiol. Plant Mol. Biol. (1997) 48:89-108, incorporated herein by reference. Other inducible promoters include plant-derived promoters, such as the promoters in the systemic acquired resistance pathway, for example, PR promoters.

Isolated: As used herein, the term “isolated” refers to a compound or entity that is 1) separated from at least some of the components with which it is normally associated (e.g., purified); 2) synthesized in vitro; and/or 3) produced or prepared by a process that involves the hand of man.

A “marker gene” is a gene encoding a selectable or screenable trait.

Naturally: The term “naturally” or “naturally-occurring”, as used herein, refers to processes, events, or things that occur in their relevant form in nature. By contrast, “not-naturally-occuring”, “artificial”, or “synthetic” refers to processes, events, or things whose existence or form involves the hand of man.

Operably linked. As used herein, operably linked refers to a relationship between two nucleic acids or two polypeptides wherein the expression of one of the nucleic acids or polypeptides is controlled by, regulated by, modulated by, etc., the other nucleic acid or polypeptide. For example, the transcription of a nucleic acid sequence is directed by an operably linked promoter sequence; post-translational processing of a nucleic acid is directed by an operably linked processing sequence; the translation of a nucleic acid sequence is directed by an operably linked translational regulatory sequence; the transport or localization of a nucleic acid or polypeptide is directed by an operably linked transport or localization sequence; and the post-translational processing of a polypeptide is directed by an operably linked processing sequence. A nucleic acid or polypeptide sequence that is operably linked to a second nucleic acid or polypeptide sequence is covalently linked, either directly or indirectly, to such a sequence, although any effective three-dimensional association is acceptable. It is noted that a single nucleic acid or polypeptide sequence can be operably linked to multiple other sequences. For example, a single promoter can direct transcription of multiple RNA species.

Percent (%) identity. In reference to polynucleotides, “percent (%) identity” is defined as the percentage of nucleotide residues in a polynucleotide sequence that are identical with the nucleotide residues in the specific nucleic acid sequence with which comparison is being made, after aligning the sequences and introducing gaps, as known in the art, to achieve the maximum percent sequence identity. In reference to polypeptides, “percent (%) identity” is defined as the percentage of amino acid residues in a polypeptide sequence that are identical with the amino acid residues in the specific polypeptide sequence with which comparison is being made, after aligning the sequences and introducing gaps, as known in the art, to achieve the maximum percent sequence identity.

Alignment can be performed in various ways known to those of skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. US Publication No. 20030211568 describes a number of suitable methods.

A “pharmaceutically active protein” aids or contributes to the condition of a subject in a positive manner when administered to a subject in a therapeutically effective amount. A pharmaceutically active protein may have healing curative or palliative properties against a disorder or disease and can be administered to ameliorate relieve, alleviate, delay onset of, reverse or lessen symptoms or severity of a disease or disorder. A pharmaceutically active protein also may have prophylactic properties and can be used to prevent or delay the onset of a disease or to lessen the severity of such disease, disorder, or pathological condition when it does emerge. Pharmaceutically active proteins include an entire protein or peptide or a pharmaceutically active fragment thereof. It also includes pharmaceutically active analogs of the protein or peptide or analogs of fragments of the protein or peptide. The term pharmaceutically active protein also refers to a plurality of proteins or peptides that act cooperatively or synergistically to provide a therapeutic benefit.

Polynucleotide encoding growth hormone: As used herein, the term “polynucleotide encoding growth hormone” refers to any nucleic acid sequence to be expressed in plant cells, as described herein that encodes growth hormone or a pharmaceutically active portion thereof. In many embodiments, the polynucleotide encoding growth hormone will be a protein-coding polynucleotide (in which case the encoded polypeptide may be referred to as a growth hormone polypeptide or growth hormone protein or a pharmaceutically active portion thereof). The polynucleotide or nucleic acid sequence may comprise DNA or RNA, and includes the sense or antisense sequence strand. Often, the polynucleotide can include sequence or sequences that are not expressed in nature in the relevant type of plant cell, or are not expressed at the level that the polynucleotide is expressed when expression is achieved by intervention of the hand of man, as described herein. In certain embodiments of the invention, the polynucleotide comprises gene sequences that are not naturally found in the relevant plant cell at all; often including gene sequences that are naturally found in other cell types or organisms. Alternatively or additionally, a polynucleotide encoding growth hormone or a pharmaceutically active portion thereof is one that is not naturally associated with the vector sequences with which it is associated according to the present invention. The word polynucleotide is used interchangeably with “nucleic acid” or “nucleic acid molecule” herein.

A “promoter,” as used herein, is a DNA sequence that directs initiation of transcription of an associated DNA sequence. The promoter region may also include elements that act as regulators of gene expression such as activators, enhancers, and/or repressors.

Purified. As used herein, “purified” means separated from one or more compounds or entities, e.g., one or more compounds or entities with which it was previously associated. A compound or entity may be partially purified, substantially purified, or pure, where it is pure when it is removed from substantially all other compounds or entities, i.e., is preferably at least about 90%, more preferably at least about 91%, 92%, 93%, 94%, 95%, 96%, 97%, 98%, 99%, or greater than 99% pure. In the context of a preparation of a nucleic acid molecule, a preparation may be considered substantially pure if the nucleic acid represents at least 50% of all nucleic acid molecules in the preparation, preferably at least 75%, yet more preferably at least 90%, or greater, as listed above, on a molecule per molecule basis, a w/w basis, or both. In the context of a preparation of a polypeptide, a preparation may be considered substantially pure if the polypeptide represents at least 50% of all polypeptides in the preparation, preferably at least 75%, yet more preferably at least 90%, or greater, as listed above, on a molecule per molecule basis, a w/w basis, or both. A partially or substantially purified nucleic acid or polypeptide may be removed from at least 50%, at least 60%, at least 70%, or at least 80%, at least 90%, etc., of the material with which it was previously associated, e.g., cellular material such as other cellular proteins and/or nucleic acids.

Recombinant. A “recombinant” molecule refers to a molecule that has been altered by the hand of man or one that is derived from (e.g., copied from) such a molecule. A recombinant polynucleotide typically contains sequences that are not found joined together in nature and/or that differ from a naturally occurring sequence. An amplified or assembled recombinant polynucleotide may be included in a suitable vector, and the vector can be used to transform a suitable cell, which may be referred to as a “recombinant cell”. The nucleotide may be expressed in the recombinant cell to produce, e.g., a “recombinant polypeptide”. A recombinant polynucleotide may serve a non-coding function (e.g., promoter, origin of replication, ribosome-binding site, etc.) as well. A recombinant nucleic acid, (e.g., a recombinant viral nucleic acid) may be a nucleic acid in which one or more sequences present in a naturally occurring molecule (e.g., a naturally occurring viral nucleic acid) has or have been deleted or replaced by a different sequence or sequences, or into which a non-native sequence has been inserted. A “recombinant polypeptide” typically contains sequences that are not found joined together in nature and/or that differ from a naturally occurring sequence. One example of a recombinant polypeptide is a fusion protein, e.g., a protein containing two or more different proteins or peptides (which may be natural or synthetic and may be portions of a naturally occurring or synthetic polypeptide). A recombinant polynucleotide that encodes a fusion protein may be created, for example, by removing the stop codon from the polynucleotide that encodes the first protein or peptide and appending a polynucleotide that encodes the second protein or peptide in frame, so that the resulting recombinant polynucleotide encodes a single recombinant polypeptide comprising the two proteins or peptides.

The term “regulatory element” or “regulatory sequence” in reference to a nucleic acid is generally used herein to describe a portion of nucleic acid that directs or increases one or more steps in the expression (particularly transcription, but in some cases other events such as splicing or other processing) of nucleic acid sequence(s) with which it is operatively linked. The term includes promoters and can also refer to enhancers and other transcriptional control elements. Promoters are regions of nucleic acid that include a site to which RNA polymerase binds before initiating transcription and that are typically necessary for even basal levels of transcription to occur. Generally such elements comprise a TATA box. Enhancers are regions of nucleic acid that encompass binding sites for protein(s) that elevate transcriptional activity of a nearby or distantly located promoter, typically above some basal level of expression that would exist in the absence of the enhancer. In some embodiments of the invention, regulatory sequences may direct constitutive expression of a nucleotide sequence (e.g., expression in most or all cell types under typical physiological conditions in culture or in an organism); in other embodiments, regulatory sequences may direct cell or tissue-specific and/or inducible expression. For example, expression may be induced by the presence or addition of an inducing agent such as a hormone or other small molecule, by an increase in temperature, etc. Regulatory elements may also inhibit or decrease expression of an operatively linked nucleic acid. Regulatory elements also may encompass sequences required for proper translation of the nucleotide sequence.

In general, a nucleic acid expression level may be determined using any available techniques for measuring mRNA or protein. Exemplary methods include Northern blotting, in situ hybridization, RT-PCR, sequencing, immunological methods such as immunoblotting, immunodetection, or fluorescence detection following staining with fluorescently labeled antibodies, oligonucleotide or cDNA microarray or membrane array, protein array analysis, mass spectrometry, etc. One convenient way to determine expression level often is to place a nucleic acid that encodes a readily detectable marker (e.g., a fluorescent or luminescent protein such as green fluorescent protein or luciferase, an enzyme such as alkaline phosphatase, etc.) in operable association with the regulatory element in an expression vector, introduce the vector into a cell type encoding growth hormone or a pharmaceutically active portion thereof or into an organism, maintain the cell or organism for a period of time, and then measure expression of the readily detectable marker, taking advantage of whatever property renders it readily detectable (e.g., fluorescence, luminescence, alteration of optical property of a substrate, etc.). Comparing expression in the absence and presence of the regulatory element indicates the degree to which the regulatory element affects expression of an operatively linked sequence.

Replicate: As used herein, “replicate” refers to the ability of a vector to generate copies inside a host cell. “Self-replicate” refers to the ability of a vector to copy itself inside a host cell. A vector that can “self-replicate” carries sufficient information in its own genetic elements that it does not need to rely on other genetic elements (e.g., those utilized by the host cell to replicate its own genome) for its replication. In general, a vector that can self-replicate typically is one that includes at least one replicase gene such as an RNA polymerase and possibly additional replicase genes such as a helicase, methyltransferase, etc. In certain instances additional sequences, typically present in cis (i.e., as part of the vector sequence), are required or can facilitate self-replication. It will be understood that a self-replicating vector will typically utilize host cell components such as nucleotides, amino acids, etc., and may be dependent on certain functions and/or enzymes of the host cell that supply such components.

“Small molecules” are typically less than about one kilodalton and are biological, organic, or even inorganic compounds (e.g., cisplatin). Examples of such small molecules include nutrients such as sugars and sugar-derivatives (including phosphate derivatives), hormones (such as the phytohormones gibberellic or absisic acid), and synthetic small molecules.

“Specifically regulatable” refers to the ability of a small molecule to preferentially affect transcription from one promoter or group of promoters, as opposed to non-specific effects, such as enhancement or reduction of global transcription within a cell.

A “sprouted seedling” or “sprout” is a young shoot from a seed or a root, preferably a recently germinated seed. In some embodiments, the sprouted seedlings of the invention are edible sprouted seedlings or sprouts (e.g., alfalfa sprouts, mung bean sprouts, radish sprouts, wheat sprouts, mustard sprouts, spinach sprouts, carrot sprouts, beet sprouts, onion sprouts, garlic sprouts, celery sprouts, rhubarb sprouts, a leaf such as cabbage sprouts, or lettuce sprouts, watercress or cress sprouts, herb sprouts such as parsley or clover sprouts, cauliflower sprouts, broccoli sprouts, soybean sprouts, lentil sprouts, edible flower sprouts such as sunflower sprouts, etc.). According to the present invention, the sprouted seedling may have developed to the two-leaf stage. Generally, the sprouts of the invention are two to fourteen days old.

“Substantially isolated” is used in several contexts and typically refers to the at least partial purification of a protein or polypeptide away from unrelated or contaminating components (for example, plant structural and metabolic proteins). Methods for isolating and purifying proteins or polypeptides are well known in the art.

“Transformation” refers to introduction of a nucleic acid into a cell, particularly the stable integration of a DNA molecule into the genome of an organism of interest.

Vector. “Vector” refers to a nucleic acid molecule capable of transporting another nucleic acid to which it has been linked and can include a plasmid, cosmid or viral vector. The vector may be capable of autonomous replication. Alternatively or additionally, a vector may provide one or more components necessary or sufficient for self-replication, or for replication or integration of another piece of nucleic acid. Vectors are typically nucleic acids, and may comprise DNA and/or RNA. In some embodiments, vectors are maintained extrachromosomally.

Viral nucleic acid: The term “viral nucleic acid,” as used herein, refers to a nucleic acid whose sequence includes at least one segment is found in a viral genome. The term can encompass both RNA and DNA forms of such nucleic acids and molecules having complementary sequences. DNA molecules identical to or complementary to viral RNA nucleic acids are considered viral nucleic acids, and RNA molecules identical to or complementary to viral DNA nucleic acids are considered viral nucleic acids, it being understood that DNA and RNA will contain T and U, respectively, at corresponding positions. A viral nucleic acid may include one or more portions of non-viral origin (e.g., part or all of a naturally occurring gene, an entirely artificial sequence, or a combination of naturally occurring and artificial sequences) and may include portion(s) from multiple different virus types.

Viral replicon: The term “viral replicon” refers to a nucleic acid molecule comprising a portion or portions (e.g., cis sequences) sufficient for replication of the nucleic acid by viral replicase genes. Typically such sequences include a recognition site for a viral polymerase, e.g., a viral RNA polymerase in the case of viral replicons based on RNA viruses.

### DETAILED DESCRIPTION OF THE INVENTION

The present invention provides plant-produced growth hormone polypeptides for delivery of growth hormone in amounts sufficient to achieve a physiologically significant response. The invention provides for effective delivery of growth hormone isolated from plants by means other than intravenous, subcutaneous, or intramuscular injection, including oral or mucosal delivery, in amounts sufficient to achieve a physiologically significant response.

Growth hormone for delivery in accordance with the invention can be produced in plants, e.g., as described in the methods provided herein, or by a variety of other methods. In certain apects, orally or mucosally delivered growth hormone may be used to treat a variety of conditions. For example, therapeutic methods provided may be useful for treatment in children and adults of disorders including, but not limited to, growth hormone deficiency, hypopituitarism, idiopathic short stature, short stature associated with Turner's syndrome, growth retardation due to chronic renal disease, neuroendocrine aging, Prader Willi Syndrome, short bowel syndrome, weight loss/wasting associated with HIV, etc. The availability of oral growth hormone formulations will increase compliance and improve patient quality of life.

The present invention encompasses the recognition that there is a need to develop expression systems for plants that present a reduced risk of environmental contamination. Thus, provided are methods and reagents for expression of polynucleotide and polypeptide products in plants with a reduced risk of widespread contamination. For example, in one aspect, the invention provides sets of viral expression vectors, each of which is incapable of establishing a systemic infection on its own, but which together allow for systemic infection. Cross-complementation (also referred to as trans-complementation) by the vectors allows an initial local infection (e.g., established by inoculation) to move into uninoculated leaves and establish a systemic infection.

In specific embodiments, the invention provides a system including a producer vector that includes a polynucleotide encoding growth hormone or a pharmaceutically active portion thereof but lacks functional versions of one or more genes necessary for long-distance movement, together with a carrier vector that provides a functional long distance movement protein coding sequence. For example, the invention provides a system for expressing a polynucleotide encoding growth hormone or a pharmaceutically active portion thereof in a plant cell or whole plant, comprising: (i) a carrier vector that includes a coat protein encoding component from a first plant virus; and (ii) a producer vector that includes a polynucleotide encoding growth hormone or a pharmaceutically active portion thereof, and further includes at least one component from a second plant virus, but lacks a functional coat protein gene. The invention further provides a system for expressing a polynucleotide encoding growth hormone or a pharmaceutically active portion thereof in a plant cell or whole plant, comprising: (i) a carrier vector that includes a movement protein encoding component from a first plant virus; and (ii) a producer vector that includes a polynucleotide encoding growth hormone or a pharmaceutically active portion thereof, and further includes at least one component from a second plant virus, but lacks a functional movement protein gene.

In certain embodiments of the invention the carrier vector is defective for replication. For instance, the producer vector may include a replicase gene (e.g., an RNA polymerase gene) and a movement protein gene (so that the vector is competent for cell-to-cell movement), but may lack a coat protein gene (so that the vector is not competent for long-distance (systemic) movement). The carrier vector may include a coat protein gene (so that the vector is competent for long-distance movement), but may lack a replicase gene (so that the vector is unable to self-replicate). Alternatively, the carrier vector might include a replicase gene (so that the vector is replication competent), and might be used with a producer vector that lacks both replication and long-distance movement capability. Preferred vectors are viral vectors.

The invention further provides a variety of vectors that can be used as compoments of the inventive system(s) or for other purposes. For example, the invention provides a vector comprising: (a) one or more components from a first plant virus; and (b) a partial or complete 3′ untranslated region from an RNA of a second plant virus. In certain embodiments of the invention the 3′ untranslated region facilitates systemic spread of the virus. The 3′ untranslated region may comprise a recognition site for complex formation with coat protein.

One advantage of the inventive system for expressing polynucleotides in plants is that it reduces or eliminates the risk that vectors, particularly recombinant vectors comprising the polynucleotide(s) to be expressed, will spread to non-target plants, thereby significantly improving the environmental safety of gene expression in plants and allowing more flexibility in the cultivation of recipient plants.

Another advantage associated with the present invention is that it allows the researcher to design a plant expression system with qualities of more than one plant virus. For instance, in certain embodiments of the invention the producer vector desirably has the polynucleotide encoding growth hormone or a pharmaceutically active portion thereof positioned such that its expression is controlled by the coat protein (“CP”) promoter. In many cases, therefore, it will be desirable to base the producer vector on a viral system with a strong CP promoter. However, viruses with strong CP promoters sometimes have limited host specificity, e.g., they may be unable to replicate and/or accomplish cell-to-cell movement or systemic movement within certain host plants. It may be desirable, therefore, to base the carrier vector on a viral system with a broad host specificity, so that the high-expressing characteristic of the viral system from which the producer vector is derived may be exploited in a host that is ordinarily inaccessible to that viral system.

The invention provides plant viruses, plant viral vector, and methods for creating plant viral vectors for use in the present invention. In other aspects, the invention also provides a variety of methods for expressing polynucleotides in plants, e.g., using the inventive vectors and systems described herein.

1. Transient Expression Systems in Plants

A. Inventive Vectors

We have prepared vector systems that include components of two heterologous plant viruses in order to achieve a system that readily infects a wide range of plant types and yet poses little or no risk of infectious spread. The expression vectors and components and methods described in this section are intended for use in known plant systems useful for expression of heterologous proteins, including those systems known in the art as well as the systems for production in clonal root systems and sprouted seedlings which are described in further detail herein.

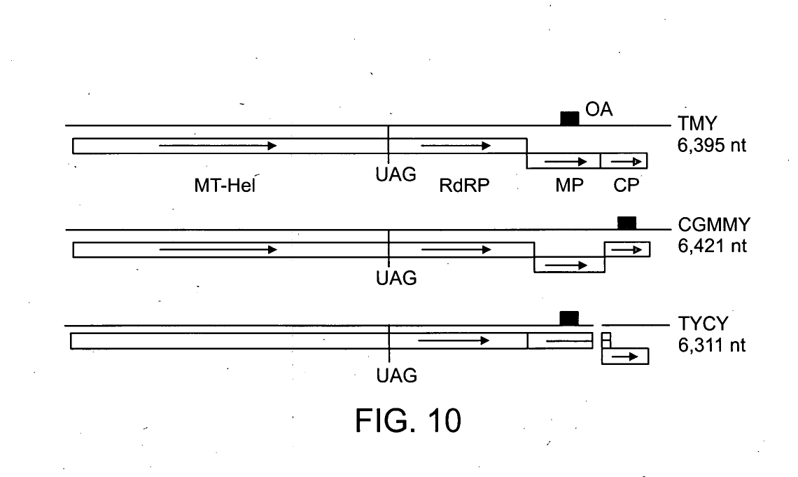

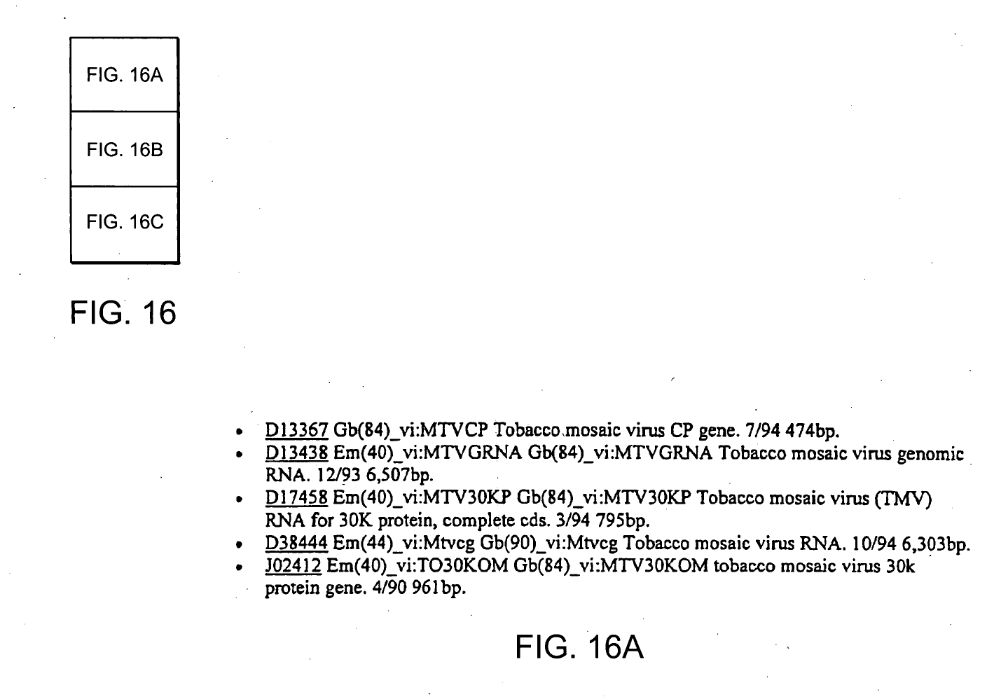

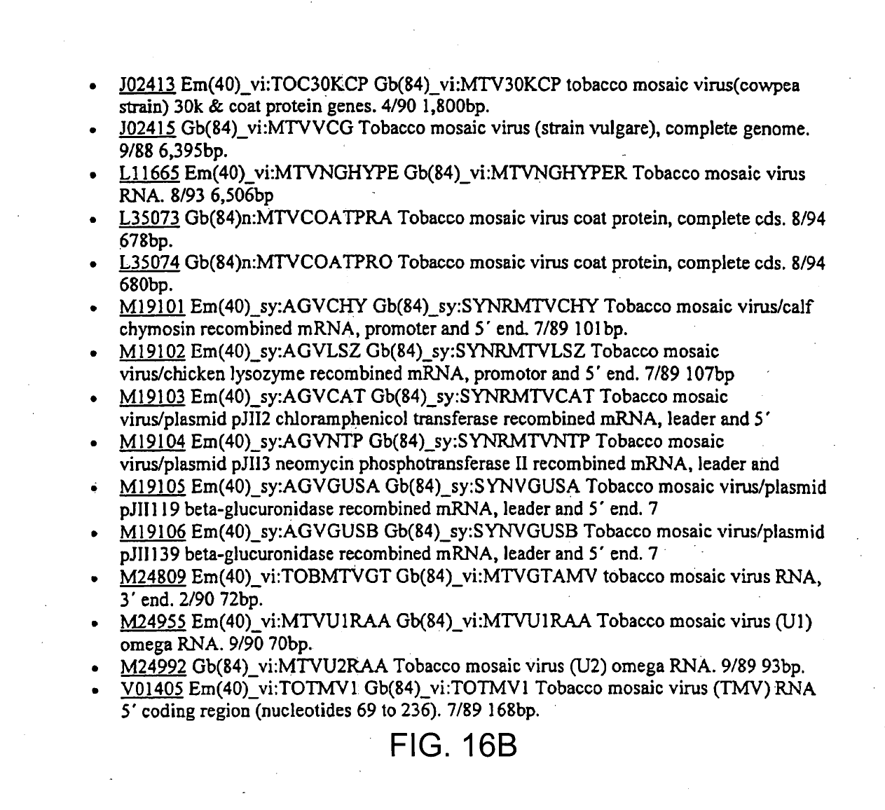

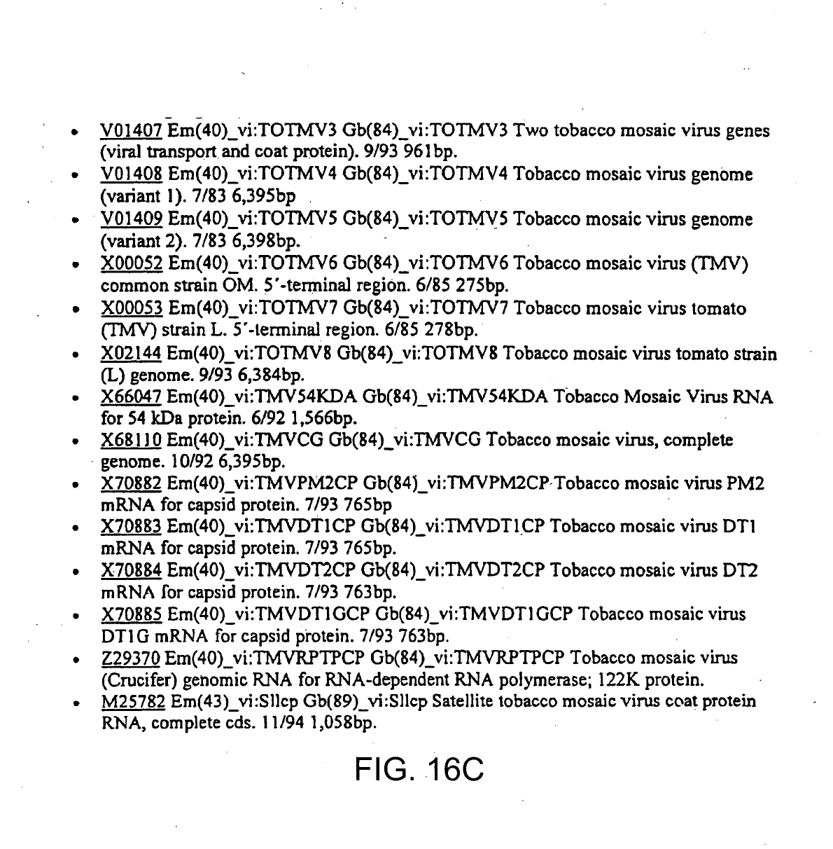

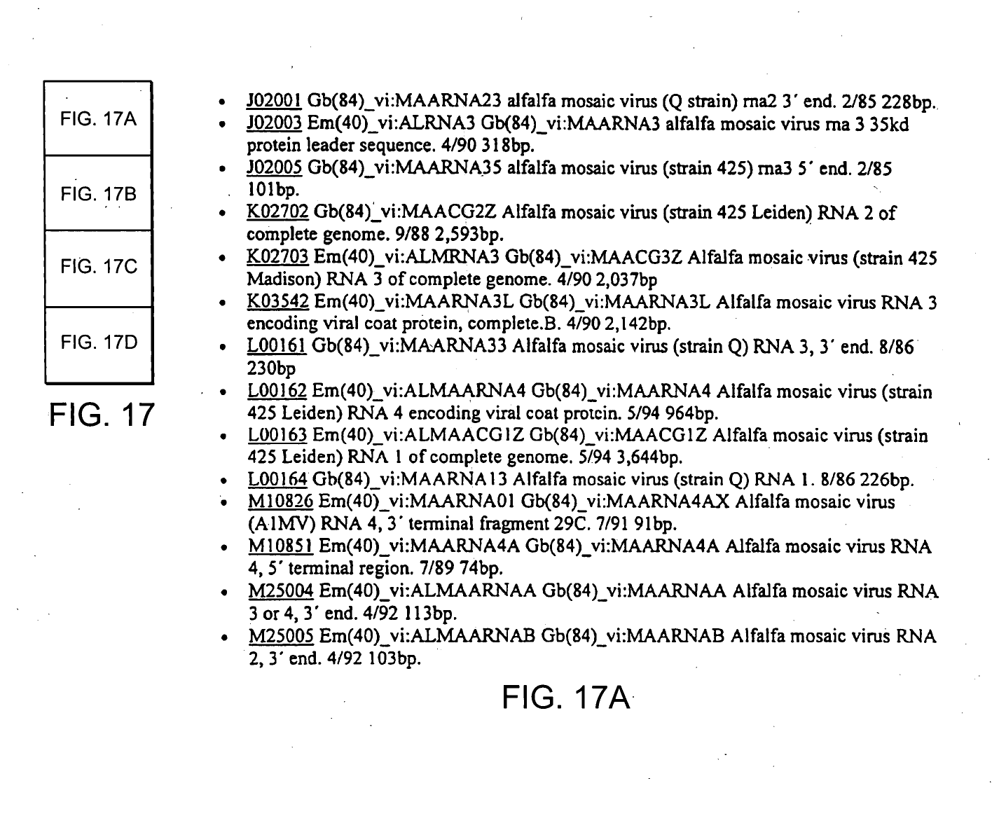

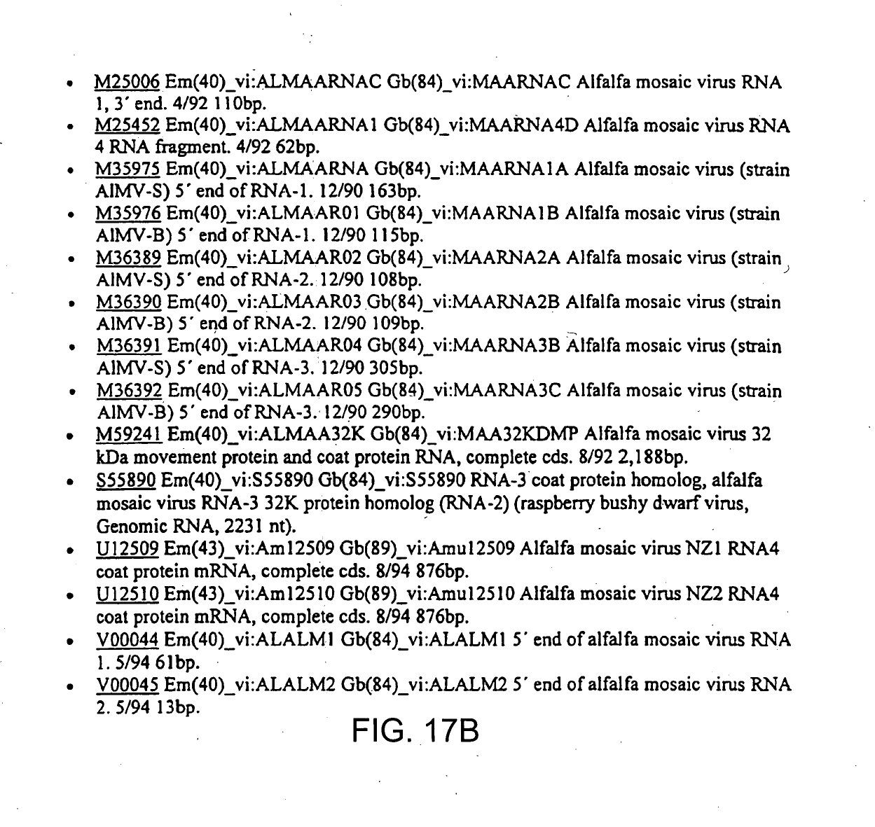

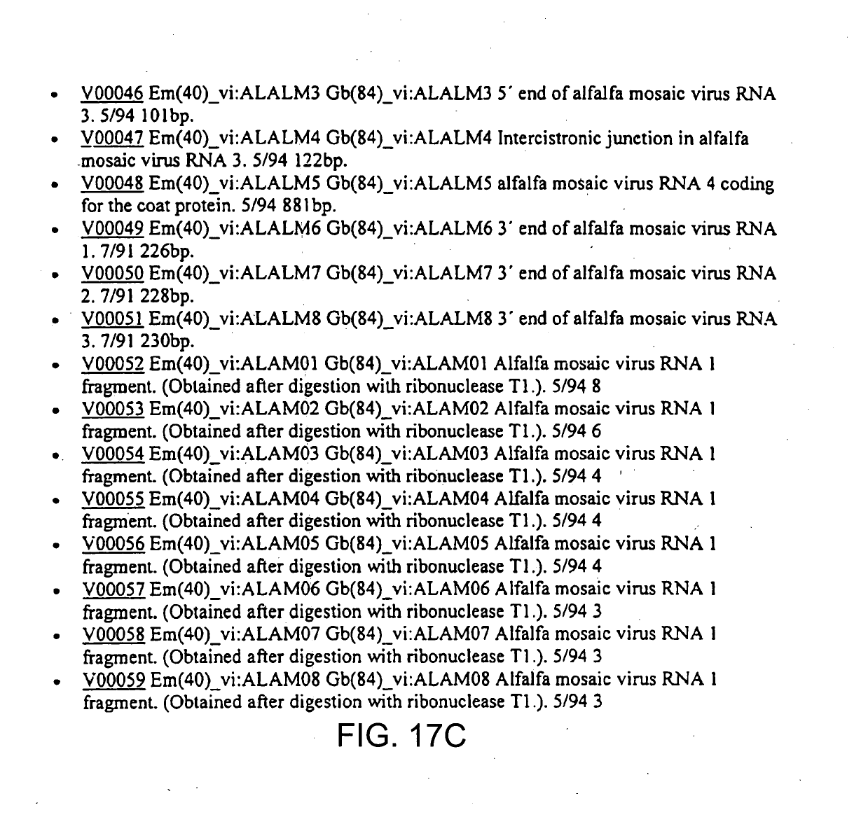

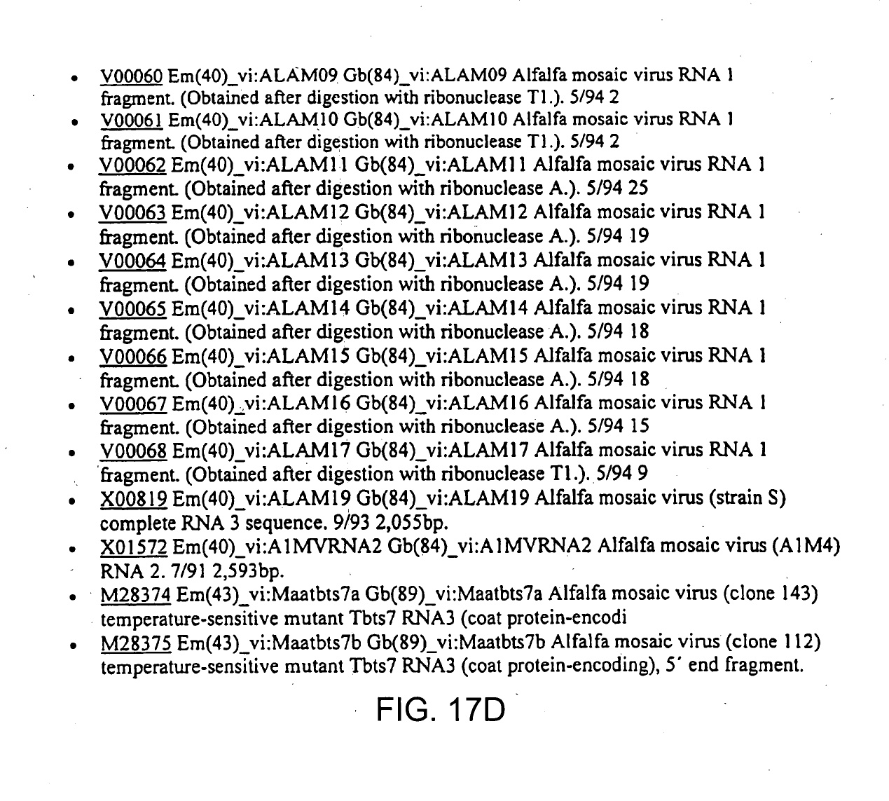

This system includes components from Alfalfa Mosaic Virus (AIMV) and Tobacco Mosaic Virus (TMV).TMV is the type member of the tobamovirus group. A representative list of accession codes for TMV genome sequence information is included as FIG. 16; Representative examples of tobamovirus genomes are depicted in FIG. 10.

According to certain embodiments of the present invention, a replication-competent version of either the AIMV or the TMV is generated that lacks long distance mobility but includes a polynucleotide to be expressed in plant tissues, preferably under control of the CP promoter (e.g., in place of the CP gene, so that CP is not functional) as the producer vector. If plants are inoculated with this vector alone, its infection is limited to local tissues (i.e., to cells within the initially infected leaf).

This replication-competent producer vector is administered together with a separate carrier vector bearing a functional CP. Transcripts of these two vectors can be mixed with one another and are mechanically applied to plant leaves. In other embodiments of the invention, the carrier vector is incompetent for replication so that no systemic infection results. The producer vector replicates and provides replicase for trans-replication of the replication-defective carrier vector. Replication of (infection with) the producer vector results in the production of the polynucleotide expression product. Replication of the carrier vector provides CP, which supports the movement of both vectors into the upper un-inoculated leaves. Integration of the vectors into the host genome can be avoided, so that transgenic plants are not produced, and the risk that genetic alterations are introduced into the environment is minimized.

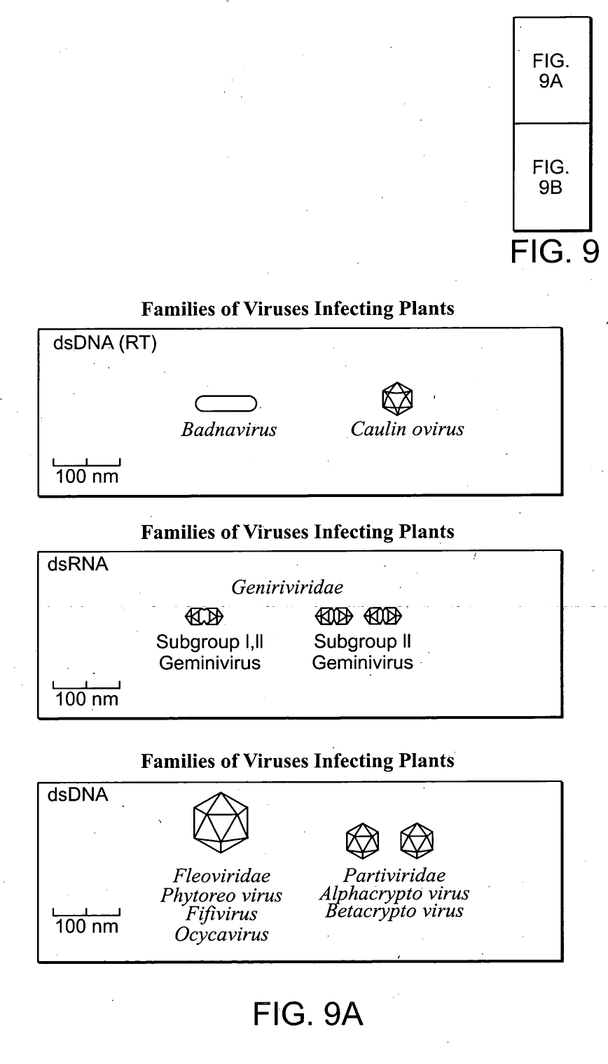

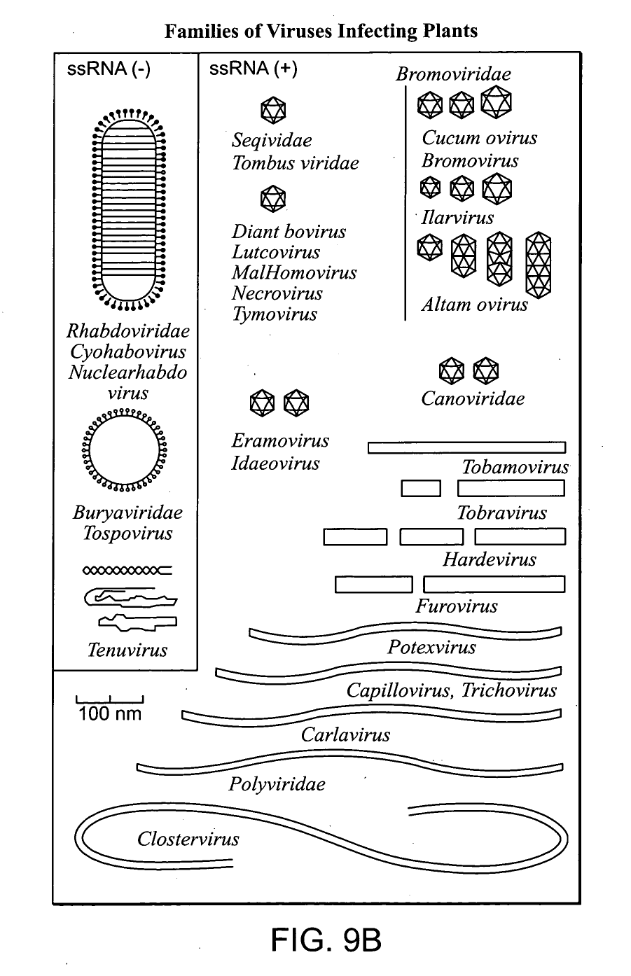

As noted above, the present invention provides systems for expressing a polynucleotide or polynucleotides encoding growth hormone or a pharmaceutically active portion thereof in plants. In certain aspect, describe in further detail herein, these systems include one or more viral vector components. A wide variety of viruses are known that infect various plant species, and can be employed for polynucleotide expression according to the present invention. FIG. 9 presents a schematic representation of certain families of viruses that infect plants. Additional information can be found, for example, in The Classification and Nomenclature of Viruses”, Sixth Report of the International Committee on Taxonomy of Viruses” (Ed. Murphy et al.), Springer Verlag: New York, 1995, the entire contents of which are incorporated herein by reference (see also, Grierson et al., Plant Molecular Biology, Blackie, London, pp. 126-146, 1984; Gluzman et al., Communications in Molecular Biology: Viral Vectors, Cold Spring Harbor Laboratory, NY, pp. 172-189, 1988; Mathew, Plant Viruses Online(http://image.fs.uidaho.edu/vide/).

In order to enter and infect a plant cell, plant viruses need to cross the cell wall, in addition to protective layers of waxes and pectins. Most or all plant viruses are thought to rely on mechanical breach of the cell wall, rather than on cell-wall-surface receptors, to enter a cell. Such a breach can be caused, for example, by physical damage to the cell, by an organism such as a bacterium, a fungus, a nematode, an insect, or a mite that can deliver the virus. In the laboratory, viruses are typically administered to plant cells simply by rubbing the virus on the plant.

Some plant viruses have segmented genomes, in which two or more physically separate pieces of nucleic acid together make up the plant genome. In some cases, these separate pieces are packaged together in the same viral capsid; in others (i.e., those with multipartite genomes), each genome segment is packaged into its own viral particle. Infection can typically be accomplished by delivery either of plant viral nucleic acid (e.g., RNA) or capsid.

Once the virus has entered (infected) a cell, it typically replicates within the infected cell and then spreads locally (i.e., from cell to cell within leaves that were infected initially). Following local spread, the virus may move into uninfected leaves, e.g., upper leaves of the plant, which is referred to as systemic infection or systemic spread. In general, cell-to-cell spread of many plant viruses requires a functional movement protein while systemic spread requires a functional coat protein (and, generally, also a functional movement protein). In addition to functional movement and coat protein encoding components, viruses may contain additional components that are either required for local or systemic spread or facilitate such spread. These cis-acting components may be either coding or noncoding components. For example, they may correspond to portions of a 3′ untranslated region (UTR, also referred to as NTR) of a viral transcript (i.e., they may provide a template for transcription of a 3′ untranslated region of a viral transcript). Thus important viral components for infection can be either coding or noncoding regions of a viral genome. By “functional protein encoding component” is meant a polynucleotide comprising a coding portion that encodes a functionally active protein, operably linked to sufficient regulatory elements such as a promoter, so that expression is achieved.

In order to successfully establish either a local (intraleaf) or systemic infection a virus must be able to replicate. Many viruses contain genes encoding one or more proteins that participate in the replication process (referred to herein as replication proteins or replicase proteins). For example, many RNA plant viruses encode an RNA polymerase. Additional proteins may also be required, e.g., helicase or methyltransferase protein(s). The viral genome may contain various sequence components in addition to functional genes encoding replication proteins, which are also required for or facilitate replication.

Any virus that infects plants may be used to prepare a viral vector or vector system in accordance with the present invention. Preferred viruses are ssRNA viruses, most desirably with a (+)-stranded genome. Techniques and reagents for manipulating the genetic material present in such viruses are well known in the art. Typically, for example, a DNA copy of the viral genome is prepared and cloned into a microbial vector, particularly a bacterial vector. Certain ssDNA viruses, including particularly geminiviruses, are also preferred. It will be appreciated that in general the vectors and viral genomes of the invention may exist in RNA or DNA form. In addition, where reference is made to a feature such as a genome or portion thereof of an RNA virus, which is present within a DNA vector, it is to be understood that the feature is present as the DNA copy of the RNA form.

Viruses of a number of different types may be used in accordance with the invention. Preferred viruses include members of the Bromoviridae (e.g., bromoviruses, alfamoviruses, ilarviruses) and Tobamoviridae. Certain preferred virus species include, for example, Alfalfa Mosaic Virus (AIMV), Apple Chlorotic Leaf Spot Virus, Apple Stem Grooving Virus, Barley Stripe Mosiac Virus, Barley Yellow Dwarf Virus, Beet Yellow Virus, Broad Bean Mottle Virus, Broad Bean Wilt Virus, Brome Mosaic Virus (BMV), Carnation Latent Virus, Carnation Mottle Virus, Carnation Ringspot Virus, Carrot Mottle Virus, Cassava Latent Virus (CLV), Cowpea Chlorotic Mottle Virus, Cowpea Mosaic Virus (CPMV), Cucumber Green Mottle Mosaic Virus, Cucumber Mosaic Virus, Lettuce Infectious Yellow Virus, Maize Chlorotic Mottle Virus, Maize Rayado Fino Virus, Maize Streak Virus (MSV), Parsnip Yellow Fleck Virus, Pea Enation Mosaic Virus, Potato Virus X, Potato Virus Y, Raspberry Bushy Dwarf Virus, Rice Necrosis Virus (RNV), Rice Stripe Virus, Rice Tungro Spherical Virus, Ryegrass Mosaic Virus, Soil-borne Wheat Mosaic Virus, Southern Bean Mosaic Virus, Tobacco Etch Virus (TEV), Tobacco Mosaic Virus (TMV), Tobacco Necrosis Virus, Tobacco Rattle Virus, Tobacco Ring Spot Virus, Tomato Bushy Stunt Virus, Tomato Golden Mosaic Virus (TGMV), and Turnip Yellow Mosaic Virus (TYMV).

Elements of these plant viruses are genetically engineered according to known techniques (see, for example, (see, for example, Sambrook et al., Molecular Cloning, 2^{nd} Edition, Cold Spring Harbor Press, NY, 1989; Clover et al., Molecular Cloning, IRL Press, Oxford, 1985; Dason et al., Virology, 172:285-292, 1989; Takamatsu et al., EMBO J. 6:307-311, 1987; French et al., Science 231: 1294-1297, 1986; Takamatsu et al., FEBS Lett. 269:73-76, 1990; Yusibov and Loesch-Fries, Virology, 208(1): 405-7, 1995. Spitsin et al., Proc Natl Acad Sci USA, 96(5): 2549-53, 1999, etc.) to generate viral vectors for use in accordance with the present invention. According to the present invention, at least two vectors are employed, one or both of which are incapable of systemic infection, but which together provide all functions needed to support systemic infection of at least one of the vectors and allow expression of a polynucleotide encoding growth hormone or a pharmaceutically active portion thereof throughout the plant. Thus the invention provides the recognition that viral components can complement each other in trans, to provide systemic infection capability.

In particular, according to the invention, a producer vector is prepared. This vector includes a polynucleotide encoding growth hormone or a pharmaceutically active portion thereof under control of regulatory sequences that direct expression in the relevant plant host. In certain aspects, the polynucleotide is placed under control of a viral promoter, for example the CP promoter. For instance, it will often be desirable to replace the natural viral CP gene with the polynucletide encoding growth hormone or a pharmaceutically active portion thereof. The producer vector lacks one or more components required for systemic movement. For example, in certain aspects of the invention the producer vector does not contain sequences sufficient for expression of functional CP (e.g., a CP gene), but may include a gene encoding a cell-to-cell movement protein. The producer vector may contain one or more sequence elements, e.g., an origin of assembly, that may be required in cis to facilitate spread of the virus when present in cis. For example, the producer vector may contain an origin of assembly that is needed for or facilitates activity of a CP, either from the same type of virus as the producer virus or from another virus. Such sequence elements may comprise a recognition site for a CP. In other embodiments of the invention the producer vector may lack sequences sufficient for expression of functional MP and/or replicase proteins. In these embodiments of the invention the producer vector may or may not lack sequences sufficient for expression of functional CP.

According to the invention, a carrier vector is also prepared. This vector complements the producer vector, i.e., it provides components needed for systemic infection that are missing in the producer vector. For example, certain carrier vectors include a functional coat protein encoding component. These carrier vectors are suitable for complementing a producer vector that lacks a functional coat protein encoding component. The carrier vector may lack at least one viral component (e.g., a gene encoding a replicase or movement protein) required for successful systemic infection of a plant, provided that such component is not also absent in the producer vector. The carrier vector may include a polynucleotide encoding growth hormone or a pharmaceutically active portion thereof (which may be the same as or different from the polynucleotide of interest in the producer vector). In such cases it may be desirable to use a carrier vector that is defective for systemic infection, e.g., because it lacks one or more necessary cis-acting sequences, in order to minimize spread of the recombinant carrier vector to non-target plants.

The carrier vector may (but need not) include a cell-to-cell movement component (e.g., a gene encoding a cell-to-cell movement protein or a noncoding component that is needed for cell-to-cell movement) and/or may lack one or more replicase protein encoding components. In those embodiments of the invention in which the carrier vector does not include a cell-to-cell movement component (e.g., a functional MP encoding portion), such a component should be included in the producer vector.

A complete inventive vector set includes all components necessary for successful systemic viral infection and expression of a polynucleotide encoding growth hormone or a pharmaceutically active portion thereof. The term “component” is intended to include both protein coding sequences and non-coding sequences such as cis-acting sequences (e.g., promoters, origin of assembly, portions corresponding to untranslated regions in mRNA). Different vectors, or vector elements, may be derived from different plant viruses (see, for example, Examples 1 and 4). In fact, as discussed herein, it will often be desirable to prepare inventive vectors from elements of different viruses in order to take advantage of different viral characteristics (e.g., host range, promoter activity level, virion dimensions, etc.).

In one aspect the invention provides a producer vector that includes a polynucleotide encoding growth hormone or a pharmaceutically active portion thereof, a replicase gene, and a movement protein gene and lacks a functional coat protein encoding component, and a carrier vector is provided that expresses a coat protein gene. For example, as described in more detail in the Examples, a producer vector may comprise a TMV-based vector in which the TMV CP coding sequence has been replaced by a polynucleotide encoding growth hormone or a pharmaceutically active portion thereof, under control of the TMV CP promoter. This producer vector is unable to move systemically. A wild type AIMV vector can serve as the carrier vector. The AIMV vector comprises a functional coat protein encoding component. Co-infection with both producer and carrier vectors allows the CP produced from the AIMV vector CP coding sequence to complement the TMV-based vector, resulting in systemic movement of the TMV-based vector and expression of the polynucleotide in leaves that were not initially infected. Alternately, an AIMV-based vector in which one or more viral components other than those required for expression of AIMV CP has been removed can be used (e.g., an AIMV-based vector lacking functional MP or replication protein coding components), provided that functional CP coding sequences and an operably linked promoter are present. The CP can be from AIMV or from another virus.

In certain embodiments of the invention the CP allows for systemic movement of the carrier vector, while in other embodiments a CP is selected that does not allow for systemic movement of the carrier vector but does allow for systemic movement of the producer vector. In those embodiments of the invention in which the carrier vector lacks one or more of the viral components other than those required for expression of AIMV CP, the producer vector may complement the carrier vector, i.e., the producer vector may supply a component such as a functional MP or replicase protein coding sequence that allows for cell-to-cell movement or replication, respectively, of the carrier vector (and, preferably, also the producer vector). It will be appreciated that where either the producer or the carrier is lacking a replication protein encoding component (e.g., a functional RNA polymerase coding component) and the other vector (carrier or producer, respectively) supplies the missing component, it will often be desirable to insert a promoter (e.g., a genomic promoter) from the vector that supplies the functional replication component into the vector lacking the functional replication protein coding component in order to achieve effective trans-complementation of replication function.

Another example of a provided viral vector system includes a producer vector in which a polynucleotide encoding growth hormone or a pharmaceutically active portion thereof is inserted into an AIMV vector, replacing the native AIMV CP encoding component. The polynucleotide encoding growth hormone or a pharmaceutically active portion thereof is placed under control of the AIMV CP promoter. This producer vector is incapable of systemic infection since it lacks CP but is able to replicate and move cell-to-cell within an infected leaf. The system also includes a cauliflower mosaic virus (CMV)-based carrier vector in which an AIMV CP encoding portion, with or without the AIMV CP 3′ UTR is inserted into a CMV vector, replacing the CMV CP encoding component found in the genome of naturally occurring CMV. The AIMV CP encoding component is placed under control of the CMV CP promoter. This vector expresses AIMV CP. Co-infection with the producer and carrier vectors allows CP expressed from the carrier vector to trans-complement the producer vector's lack of functional CP encoding components, allowing systemic movement of the producer vector. The AIMV CP also allows systemic movement of the carrier vector.

In certain embodiments of the invention it is desirable to insert a portion of coding or noncoding sequence from the carrier vector into the producer vector, or vice versa. For example, certain sequences may enhance replication or facilitate cell-to-cell or long distance movement. In particular, certain sequences may serve as recognition sites for formation of a complex between a viral transcript and a CP (e.g., an origin of assembly). In such a case, if systemic movement of a first viral vector is to be achieved using CP provided in trans from a second viral vector, it may be desirable to insert such sequences from the second viral vector that facilitate activity of the CP into the first viral vector. Such sequences may comprise, for example, part or all of a viral transcript 3′ UTR. As described in Example 4, in certain embodiments of the invention part or all of the RNA3 3′ UTR of AIMV is inserted into a different viral vector, e.g., a TMV-based vector. Including this component in the TMV-based vector facilitates the ability to AIMV CP to trans-complement a TMV-based vector that lacks a functional TMV CP encoding portion. It will be appreciated that this general principle may be applied to any viral vector system comprising trans-complementing vectors, e.g. trans-complementing producer and carrier vector systems.

As will be appreciated by those of ordinary skill in the art, so long as a vector set includes a producer vector that is incapable of systemic viral infection (i.e., lacking one or more functional replication protein, movement protein, or coat protein encoding components) and a carrier vector that provides the function(s) lacking in the producer vector, that set is appropriate for use in accordance with the present invention. In certain embodiments of the invention no individual vector is capable of systemic viral infection but, as a set, one or both of the vectors is competent for such infection and expression of the polynucleotide encoding growth hormone or a pharmaceutically active portion thereof. Such a system offers a number of advantages. For example, it will be appreciated that if the producer vector infects a plant in the absence of the carrier vector, no systemic infection will result. This diminishes the risk that the polynucleotide encoding growth hormone or a pharmaceutically active portion thereof will be expressed in unintended (non-target) plants, even of the same species as the target plant. In particular, if the carrier vector is not competent for replication or cell-to-cell movement (because it lacks a component required for replication or cell-to-cell movement) or if it is incompetent for systemic infection (e.g., because it lacks a cis-acting sequence such as an origin of assembly that is required for long distance movement), the likelihood that both producer and carrier vectors will co-infect an unintended plant host are greatly reduced.

Generally, in order to preserve viral function and also simply for ease of genetic manipulation, inventive vectors will be prepared by altering an existing plant virus genome, for example by removing particular genes and/or by disrupting or substituting particular sequences so as to inactivate or replace them. In such circumstances, the inventive vectors will show very high sequence identity with natural viral genomes. Of course, completely novel vectors may also be prepared, for example, by separately isolating individual desired genetic elements and linking them together, optionally with the inclusion of additional elements. Also, it should be noted that where a particular vector is said to lack a given gene, protein, or activity (e.g., the producer vector lacks a coat protein gene), it is sufficient if no such protein or activity is expressed from the vector under conditions of infection, even though the vector may still carry the relevant coding sequence. In general, however, it is typically desirable to remove the relevant coding sequences from the vector.

Analogously, when an inventive vector is said to affirmatively express a particular protein or activity, it is not necessary that the relevant gene be identical to the corresponding gene found in nature. For instance, it has been found that the coat protein can sometimes tolerate small deletions (see, for example WO 00/46350, incorporated herein by reference). So long as the protein is functional, it may be used in accordance with the present invention. Very high sequence identity with the natural protein, however, is generally preferred. For instance, large deletions (e.g., greater than about 25 amino acids) should generally be avoided according to certain embodiments of the invention. Typically, viral proteins expressed in accordance with the present invention will show at least 50%, preferably 60%, 70%, 80%, 85%, 90%, 91%, 92%, 93%, 94%, 95%, 96%, 97%, 98%, or 99% sequence identity with the corresponding natural viral protein. More particularly, the inventive viral protein should typically show 100% identity with critical functional portions (typically of at least several amino acids, often of at least 10, 20, 30, 40, 50 or more amino acids) of the relevant natural viral protein.

It is noted that in the case of many proteins a number of amino acid changes can be made without significantly affecting the functional activity and/or various other properties of the protein such as stability, etc. In particular, many proteins tolerate conservative amino acid changes, i.e., the substitution of an amino acid with a different amino acid having similar properties (conservative substitution) at many positions without significant reduction in activity. Conservative amino acid substitution is well known in the art and represents one approach to obtaining a polypeptide having similar or substantially similar properties to those of a given polypeptide while altering the amino acid sequence. In general, amino acids have been classified and divided into groups according to (I) charge (positive, negative, or uncharged); (2) volume and polarity; (3) Grantham's physico-chemical distance; and combinations of these. See, e.g., Zhang, J., J. Mol. Evol., 50: 56-68, 2000; Grantham R., Science, 85: 862-864, 1974; Dagan, T., et al., Mol. Biol. Evol., 19(7), 1022-1025, 2002; Biochemistry, 4th Ed., Stryer, L., et al., W. Freeman and Co., 1995; and U.S. Pat. No. 6,015,692. For example, amino acids may be divided into the following 6 categories based on volume and polarity: special (C); neutral and small (A, G, P, S, T); polar and relatively small (N, D, Q, E), polar and relatively large (R, H, K), nonpolar and relatively small (I, L, M, V), and nonpolar and relatively large (F, W, Y). A conservative amino acid substitution may be defined as one that replaces one amino acid with an amino acid in the same group. Thus a variety of functionally equivalent proteins can be derived by making one or more conservative amino acid substitutions in a given viral protein.

B. Plant Viruses

A wide variety of viruses are known that infect various plant species, and can be employed for polynucleotide expression according to the present invention. FIG. 9 presents a schematic representation of certain families of viruses that infect plants, based on the type of nucleic acid (e.g., dsDNA, ssDNA, ssRNA, dsRNA, or unassigned) that makes up the viral genome. Additional information can be found, for example, in The Classification and Nomenclature of Viruses”, Sixth Report of the International Committee on Taxonomy of Viruses” (Ed. Murphy et al.), Springer Verlag: New York, 1995, the entire contents of which are incorporated herein by reference (see also, Grierson et al., Plant Molecular Biology, Blackie, London, pp. 126-146, 1984; Gluzman et al., Communications in Molecular Biology: Viral Vectors, Cold Spring Harbor Laboratory, NY, pp. 172-189, 1988; Mathew, Plant Viruses Online (http://image.fs.uidaho.edu/vide/).

In nature, in order to enter and infect a plant cell, plant viruses need to cross the cell wall, in addition to protective layers of waxes and pectins. Most or all plant viruses are thought to rely on mechanical breach of the cell wall, rather than on cell-wall-surface receptors, to enter a cell. Such a breach can be caused, for example, by physical damage to the cell, by an organism such as a bacterium, a fungus, a nematode, an insect, or a mite that can deliver the virus. In the laboratory, viruses are typically administered to plant cells simply by rubbing the virus on the plant.

Some plant viruses have segmented genomes, in which two or more physically separate pieces of nucleic acid together make up the plant genome. For example, many RNA plant virus genomes can be classified as mono-, bi-, or tri-partite, i.e., they may consist of 1, 2, or 3 nucleic acids respectively. In some cases, these separate pieces are packaged together in the same viral capsid; in others (i.e., those with multipartite genomes), each genome segment is packaged into its own viral particle. Infection can typically be accomplished by delivery either of plant viral nucleic acid (e.g., RNA) or capsid containing such nucleic acid.

Once the virus has entered (infected) a cell, it typically replicates within the infected cell and then spreads locally (i.e., from cell to cell within leaves that were infected initially). Following local spread, the virus may move into uninfected leaves, e.g., upper leaves of the plant, which is referred to as systemic infection or systemic spread. In general, cell-to-cell spread of many plant viruses requires a functional movement protein (which allows movement of viral transcripts) while systemic spread requires a functional coat protein (and, generally, also a functional movement protein), which allows the formation of viral particles.

In addition to functional movement and coat protein encoding components, the viral genome may contain additional components that are required for local (e.g., cell-to-cell) or long distance (e.g., systemic) spread or facilitate such spread. These cis-acting components may be either coding or noncoding components. For example, they may correspond to portions of a 3′ untranslated region (UTR, also referred to as NTR) of a viral transcript (i.e., they may provide a template for transcription of a 3′ untranslated region of a viral transcript). Thus important viral components can be either coding or noncoding regions of a viral genome and include a variety of regulatory regions. Such regions may function in replication and/or processing or expression of mRNA. By “functional protein encoding component” is meant a polynucleotide comprising a coding portion that encodes a functionally active protein, operably linked to sufficient regulatory elements such as a promoter, so that expression is achieved.

In order to successfully establish either a local (intraleaf) or systemic infection a virus must be able to replicate. Many viruses contain genes encoding one or more proteins that participate in the replication process (referred to herein as replication proteins or replicase proteins). For example, many RNA plant viruses encode an RNA polymerase. Additional proteins may also be required, e.g., helicase or methyltransferase protein(s). The viral genome or segment may contain various sequence components, e.g., cis-acting sequences, in addition to functional genes encoding replication proteins, which are also required for or facilitate replication. Viral genomes or segments may also contain cis-acting sequences that contribute to high levels of transcript and/or expression. It is noted that nucleic acids encoding various viral proteins, e.g., replicase proteins, movement protein, coat protein, may be present within different viral nucleic acid molecules, which may complement each other in trans. (See, e.g., WO 00/25574 and co-pending U.S. National application Ser. No. 10/770,600, entitled “SYSTEM FOR EXPRESSION OF GENES IN PLANTS”, filed Feb. 3, 2004. Thus in certain embodiments of the invention rather than delivering a single viral vector to a plant cell, multiple different vectors are delivered which, together, allow for replication (and, optionally cell-to-cell and/or long distance movement) of the viral vector(s). Some or all of the proteins may be encoded by the genome of transgenic plants.

Viral vectors based on any virus that infects plants may be used to generate a clonal root line, clonal plant cell line or clonal plant that expresses a polynucleotide encoding growth hormone or a pharmaceutically active portion thereof in accordance with the present invention. In certain aspects, viruses are ssRNA viruses, most desirably with a (+)-stranded genome. Techniques and reagents for manipulating the genetic material present in such viruses are well known in the art. Typically, for example, a DNA copy of the viral genome is prepared and cloned into a microbial vector, particularly a bacterial vector. Certain ssDNA viruses, including particularly geminiviruses, may also be used. It will be appreciated that in general plant viral vectors and viral nucleic acids such as viral genomes may exist in RNA or DNA form. In addition, where reference is made to a feature such as a genome or portion thereof of an RNA virus, which is present within a DNA vector, it is to be understood that the feature is present as the DNA copy of the RNA form.

Vectors may be based on viruses such as members of the Bromoviridae (e.g., bromoviruses, alfamoviruses, ilarviruses) and Tobamoviridae. Certain preferred virus species include, for example, Alfalfa Mosaic Virus (AIMV), Apple Chlorotic Leaf Spot Virus, Apple Stem Grooving Virus, Barley Stripe Mosiac Virus, Barley Yellow Dwarf Virus, Beet Yellow Virus, Broad Bean Mottle Virus, Broad Bean Wilt Virus, Brome Mosaic Virus (BMV), Carnation Latent Virus, Carnation Mottle Virus, Carnation Ringspot Virus, Carrot Mottle Virus, Cassaya Latent Virus (CLV), Cowpea Chlorotic Mottle Virus, Cowpea Mosaic Virus (CPMV), Cucumber Green Mottle Mosaic Virus, Cucumber Mosaic Virus, Lettuce Infectious Yellow Virus, Maize Chlorotic Mottle Virus, Maize Rayado Fino Virus, Maize Streak Virus (MSV), Parsnip Yellow Fleck Virus, Pea Enation Mosaic Virus, Potato Virus X, Potato Virus Y, Raspberry Bushy Dwarf Virus, Rice Necrosis Virus (RNV), Rice Stripe Virus, Rice Tungro Spherical Virus, Ryegrass Mosaic Virus, Soil-borne Wheat Mosaic Virus, Southern Bean Mosaic Virus, Tobacco Etch Virus (TEV), Tobacco Mosaic Virus (TMV), Tobacco Necrosis Virus, Tobacco Rattle Virus, Tobacco Ring Spot Virus, Tomato Bushy Stunt Virus, Tomato Golden Mosaic Virus (TGMV), and Turnip Yellow Mosaic Virus (TYMV).

In certain embodiments of the invention a TMV-based viral vector (viral nucleic acid) is used. TMV is the type member of the tobamovirus group. Tobamoviruses have single-(+)-stranded RNA genomes, and produce rod-shaped virions consisting of the RNA genome and coat protein (CP) polypeptides. Tobamovirus genomes encode 4-5 polypeptides. Two of the polypeptides are translated from the same 5′-proximal initiation codon and function in viral replication. These polypeptides include an RNA-dependent RNA polymerase. In addition, polypeptides having methyltransferase and RNA helicase activity are typically encoded. The other encoded proteins typically include a movement protein and the coat protein, each of which is translated from a separate subgenomic RNA. Representative examples of tobamovirus genomes are depicted in FIG. 16. Tobamoviruses other than TMV can be used in various embodiments of the invention.

The TMV genome is 6395 nucleotides long and is encapsidated with a 17.5 kD CP, which produces 300 nm-long rods. In addition to CP, TMV has three nonstructural proteins: 183 and 126 kD proteins are translated from genomic RNA and are required for viral replication. The 30 kD movement protein provides for the transfer of viral RNA from cell-to-cell. Plant species susceptible to infection with TMV include Beta vulgaris, Capsicum frutescens, Chenopodium amaranticolor, Chenopodium hybridum, Chenopodium quinoa, Cucumis melo, Cucumis sativus, Cucurbita pepo, Datura stramonium, Lactuca sativa, Lucopersicon esculentum, Lycopersicon pimpinellifolium, Nicotiana benthamiana, Nicotiana bigelovii, Nicotiana clevelandii, Nicotiana debneyi, Nicotiana glutinosa, Nicotiana rustica, Nicotiana sylvestris, Nicotiana tabacum, Papaver nudicaule, Phaseolus vulgaris, Physalis floridana, Physalis peruviana, and Solanum tuberosum.

In various other embodiments of the invention an AIMV-based viral vector (viral nucleic acid) is used. AIMV is an Alfamovirus, closely related to the Ilarvirus group and is a member of the Bromoviridae family. The genome of AIMV consists of three positive-sense RNAs (RNAs 1-3). RNAs 1 and 2 encode replicase proteins P1 and P2, respectively; RNA3 encodes the cell-to-cell movement protein P3. A subgenomic RNA, RNA4, is synthesized from RNA3. This subgenomic RNA4 encodes the viral coat protein (CP). CP participates in viral genome activation to initiate infection, RNA replication, viral assembly, viral RNA stability, long-distance movement of viral RNA, and symptom formation. AIMV depends on a functional P3 protein for cell-to-cell movement, and requires the CP protein throughout infection. Depending on the size of the CP-encapsidated viral RNA, virions of AIMV can vary significantly in size (e.g., 30- to 60-nm in length and 18 nm in diameter) and form (e.g., spherical, ellipsoidal, or bacilliform).

The host range of AIMV is remarkably wide and includes the agriculturally valuable crops alfalfa (Medicago sativa), tomato (Lycopersicon esculentum), lettuce (Lactuca sativa), common bean (Phaseolus vulgaris), potato (Solanum tuberosum), white clover (Trifolium repens) and soybean (Glycine max). Particular susceptible host species include, for example, Abelmoschus esculentus, Ageratum convzoides, Amaranthus caudatus, Amaranthus retroflexus, Antirrhinum majus, Apium graveolens, Apium graveolens var. rapaceum, Arachis hypogaea, Astragalus glycyphyllos, Beta vulgaris, Brassica campestris ssp. rapa, Calendula officinalis, Capsicum annuum, Capsicum frutescens, Caryopteris incana, Catharanthus roseus, Celosia argentea, Cheiranthus cheiri, Chenopodium album, Chenopodium amaranticolor, Chenopodium murale, Chenopodium quinoa, Cicer arietinum, Cichorium endiva, Coriandrum sativum, Crotalaria spectabilis, Cucumis melo, Cucumis sativus, Cucurbita pepo, Cyamopsis tetragonoloba, Daucus carota (var. sativa), Dianthus barbatus, Dianthus caryophyllus, Emilia sagittata, Fagopyrum esculentum, Gomphrena globosa, Helianthus annuus, Lablab purpureus, Lathyrus odoratus, Lens culinarisi, Linum usitatissimum, Lupinus albus, Macroptilium lathyroides, Malva parviflora, Matthiola incana, Medicago hispida, Melilotus albus, Nicotiana bigelovii, Nicotiana clevelandii, Nicotiana debneyi, Nicotiana glutinosa, Nicotiana megalosiphon, Nicotiana rustica, Nicotiana sylvestris, Nicotiana tabacum, Ocimum basilicum, Petunia×hybrida, Phaseolus lunatus, Philadelphus, Physalis floridana, Physalis peruviana, Phytolacca americana, Pisum sativum, Solanum demissum, Solanum melongena, Solanum nigrum, Solanum nodiflorum, Solanum rostratum, Sonchus oleraceus, Spinacia oleracea, Stellaria media, Tetragonia tetragonioides, Trifolium dubium, Trifolium hybridum, Trifolium incarnatum, Trifolium pratense, Trifolium subterraneum, Tropaeolum majus, Viburnum opulus, Vicia faba, Vigna radiata, Vigna unguiculata, Vigna unguiculata ssp. sesquipedalis, and Zinnia elegans.

While AIMV is a one viral vector of use in the provided invention, other viruses, (e.g., other alfamoviruses) can also be used in various aspects of the invention, including but not limited t, related viruses, such as ilarviruses can also be used.

C. Creation of Plant Viral Expression Vectors

In accordance with the invention, elements of these plant viruses may be genetically engineered according to known techniques (see, for example, (see, for example, Sambrook et al., Molecular Cloning, 2^{nd} Edition, Cold Spring Harbor Press, NY, 1989; Clover et al., Molecular Cloning, IRL Press, Oxford, 1985; Dason et al., Virology, 172:285-292, 1989; Takamatsu et al., EMBO J. 6:307-311, 1987; French et al., Science 231: 1294-1297, 1986; Takamatsu et al., FEBS Lett. 269:73-76, 1990; Yusibov and Loesch-Fries, Virology, 208(1): 405-7, 1995. Spitsin et al., Proc Natl Acad Sci USA, 96(5): 2549-53, 1999, etc.) to generate viral vectors for use in accordance with the present invention. In general, a viral vector is a viral nucleic acid. Typically the viral vector is the genome, or a majority thereof (i.e., at least 50% of the genome), of a virus, or a nucleic acid molecule complementary in base sequence to such a nucleic acid molecule. In the case of segmented viruses, the viral vector may be a genome segment, or a majority thereof. The viral vector may be in RNA or DNA form.

The viral vector may comprise a portion sufficient to support replication of the viral vector in the presence of the appropriate viral replicase proteins, i.e., constitutes a viral replicon. The ability of any particular portion of a viral genome to support replication of a nucleic acid that includes the portion, in the presence of viral replicase proteins, can readily be tested using methods known in the art, e.g., by making deletion mutants, by transferring the portion into a nucleic acid that does not support replication and determining whether replication occurs, etc. The replicase proteins may be encoded by the vector, by another vector, or by a plant into which the vector is introduced. In certain aspects, the vector is capable of self-replication, i.e., it encodes the necessary viral proteins for replication of the virus within an appropriate plant host. In certain embodiments of the invention the vector comprises a MP gene. In certain embodiments of the invention the vector comprises a CP gene. However, in certain embodiments of the invention neither an MP gene nor a CP gene is present in the vector. Since the clonal root lines, clonal plant lines, and clonal plants are derived from single ancestral cells into which the vector has been introduced, it is not necessary for the viral vector to have cell-to-cell or long distance movement capability. In particular, clonal plants can express the polynucleotide encoding growth hormone or a pharmaceutically active portion thereof throughout the plant even though the viral transcript does not move, since each cell is derived from a single ancestral cell that contains the viral vector.

In general, a polynucleotide encoding growth hormone or a pharmaceutically active portion thereof is inserted into a viral vector under control of (i.e., operably linked to), a promoter that directs transcription of the polynucleotide in a plant cell encoding growth hormone or a pharmaceutically active portion thereof. In certain aspects a plant viral promoter is used, e.g., a promoter for coat protein, movement protein, etc. The polynucleotide encoding growth hormone or a pharmaceutically active portion thereof may be inserted in place of the endogenous MP or CP coding sequence. For example, as described in more detail in the Examples, a TMV-based vector in which the TMV CP coding sequence has been replaced by a polynucleotide encoding growth hormone or a pharmaceutically active portion thereof, under control of the TMV CP promoter can be used. Alternately, the inserted polynucleotide may include its own promoter, which may be identical or similar to one of the naturally occurring viral promoters, may be from a different virus (e.g., the cauliflower mosaic virus), may be a non-viral promoter such as a promoter for a plant gene, or a synthetic promoter. In certain embodiments of the invention an inducible promoter is used. A variety of inducible promoters are known that function in plants. See, e.g., Zuo, J. and Chua, N—H., “Chemical-inducible systems for regulated expression of plant genes”, Curr. Op. in Biotechnol., 11:146-51, 2000. For example, promoters inducible by metals such as copper, or responsive to hormones such as estrogen, or systems responsive to other small molecules such as tetracycline can be used. Other stimuli such as heat, light, etc., can be used. See U.S. Ser. No. 10/294,314.

In certain embodiments of the invention in any of its aspects, trans-activation is used to induce or increase expression of a polynucleotide encoding growth hormone or a pharmaceutically active portion thereof. For example, the expression cassette comprising the polynucleotide can be an inactive expression cassette that comprises an inactive or silenced foreign nucleic acid sequence, which is capable of directing expression of a polynucleotide encoding growth hormone or a pharmaceutically active portion thereof upon its activation. In certain embodiments of the invention trans-activation is accomplished by introducing a factor for activating or facilitating the expression of an inactive or silenced polynucleotide sequence into cells of the clonal entity. A promoter that can be activated in trans in such a manner is referred to as being “trans-activatable”. See U.S. Ser. No. 10/832,603, entitled “Expression of Foreign Sequences in Plants Using Trans-Activation System”, which is incorporated herein by reference, for further details of certain suitable methods. Such methods include techniques based on recombination (e.g., using a Lox/Cre or Flp/Frt recombinase system) and techniques based on proteins comprising a DNA binding domain such as GAL4 and a transcriptional activation domain such as VP16. A variety of other methods may be used for achieving trans-activation.

In certain embodiments of the invention the polynucleotide is inserted to create an independent open reading frame, while in other embodiments of the invention the polynucleotide is inserted to create an open reading frame in which a polynucleotide lacking a stop codon is inserted in frame with sequences encoding part or all of a viral protein such as CP, so that a fusion protein is produced upon translation. Multiple polynucleotides can be inserted. In certain aspects, the TMV vector retains part or all of its 3′ UTR and/or all or part of the CP coding sequence. In certain embodiments of the invention the polynucleotide encoding growth hormone or a pharmaceutically active portion thereof or a viral vector into which the polynucleotide encoding growth hormone or a pharmaceutically active portion thereof is inserted comprises a portion encoding a targeting sequence, e.g., a sequence that targets an encoded polypeptide to a particular intracellular organelle or compartment. For example, it may be desirable to target a growth hormone or a pharmaceutically active portion thereof polypeptide to the endoplasmic reticulum, which may ultimately result in secretion of the polypeptide. The secreted polypeptide can then be harvested from culture medium or from interstitial fluid of a plant tissue.

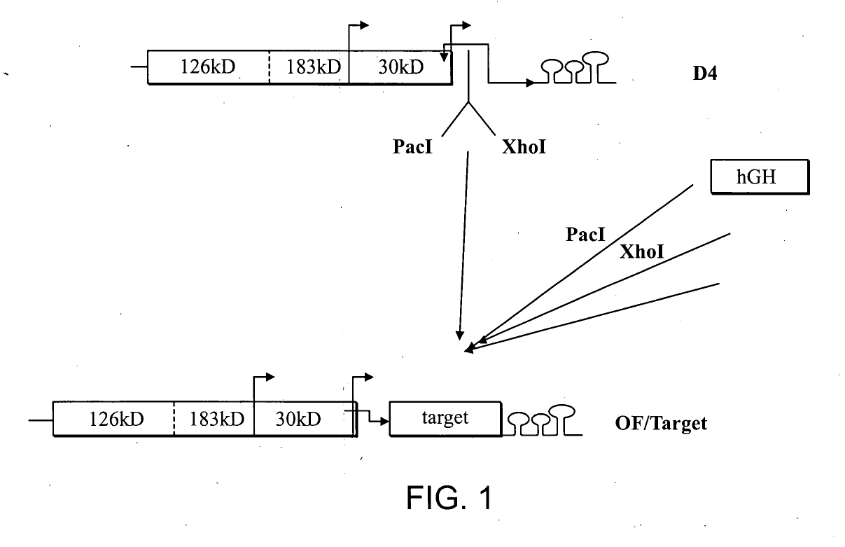

FIGS. 1-5 show examples of engineering various plant virus vectors suitable for use in the present invention. FIG. 1 shows a TMV based virus construct, D4, and the same construct following insertion of a polynucleotide encoding growth hormone (e.g., a gene encoding hGH, etc., indicated as “target”) whose transcription is under control of the TMV CP subgenomic promoter. Details regarding the creation of such vectors are given in the Examples.

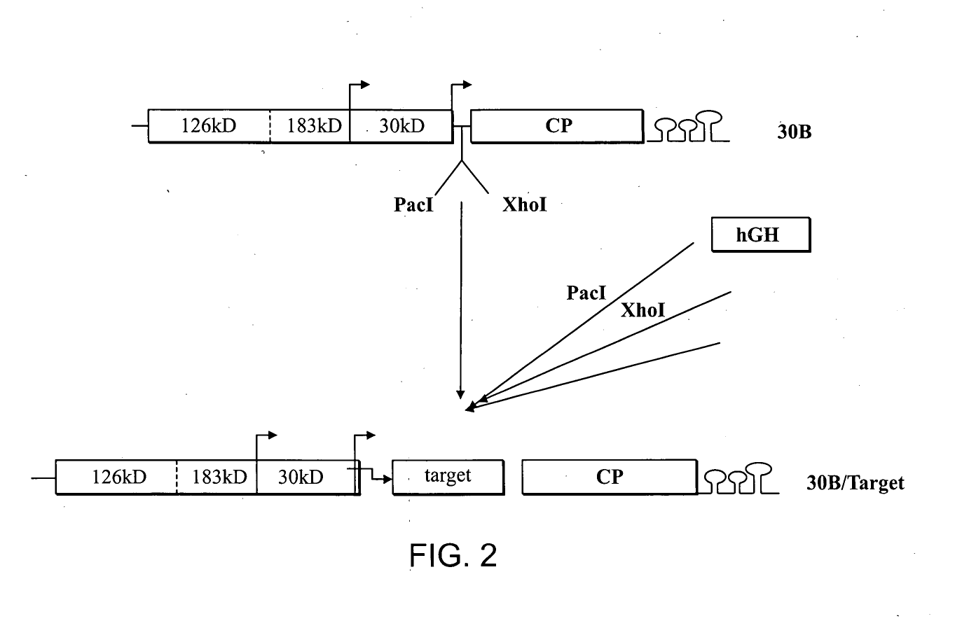

FIG. 2 presents a schematic diagram of the engineering of a TMV based viral construct containing a polynucleotide encoding growth hormone. The upper portion of the figure shows the genomic organization of a TMV based virus construct, 30B (Yusibov, V., Shivprasad, S., Turpen, T. H., Dawson, W., and Koprowski, H., “Plant viral vectors based on tobamoviruses”, in Plant Biotechnology. New Products and Applications (Eds. J. Hammond, P. McGarvey, and V. Yusibov), pp. 81-94, Springer-Verlag, 1999). The lower portion shows the same construct following insertion of a polynucleotide encoding growth hormone (e.g., a gene encoding hGH, etc., indicated as “target”). The 126/183 kDa protein is required for replication of the virus. The 30 kD protein is the movement protein (MP) that mediates cell-to-cell movement. CP is the coat protein that mediates systemic spread. Arrows indicate positions of the subgenomic promoters in certain embodiments of the invention. Transcription of the inserted polynucleotide is under control of an introduced promoter. CP expression is under control of the endogenous CP promoter in the construct shown in FIG. 2.

Similar vectors in which polynucleotide encoding growth hormone is in frame with the CP coding sequence so as to encode a fusion protein can also be used. In general, polynucleotides encoding growth hormone or a pharmaceutically active portion thereof (and their encoded proteins) can be expressed as independent open reading frames (see, e.g., Pogue, G. P., Lindbo, J. A., Dawson, W. O., and Turpen, T. H. “Tobamovirus transient expression vectors: tools for plant biology and high-level expression of foreign proteins in plants”, Pl. Mol. Biol. Manual. L4, 1-27., 1998) or as fusions with coat protein (Yusibov, V., Modelska, A., Steplewski, K., Agadjanyan, M., Weiner, D., Hooper, C. and Koprowski, H., “Antigens produced in plants by infection with chimeric plant viruses immunize against rabies virus and HIV-1”, Proc. Natl. Acad. Sci. USA 94, 5784-5788, 1997). In the vector described in the latter, target sequences are replicated from a second subgenomic promoter. In general, transcription of a polynucleotide encoding growth hormone or a pharmaceutically active portion thereof and/or an endogenous gene such as MP or CP can be driven by endogenous promoters or inserted promoters (which may be identical to naturally occurring vectors from the same or a different virus or may be synthetic, or a combination of natural and synthetic sequences.

The 3′ portion of the construct can include the TMV 3′ UTR, which may form stem-loop structure(s) as shown. The 3′ portion of the construct may also include TMV coat protein sequences that contain a cis element that may be required for optimal replication. This sequence is optional.

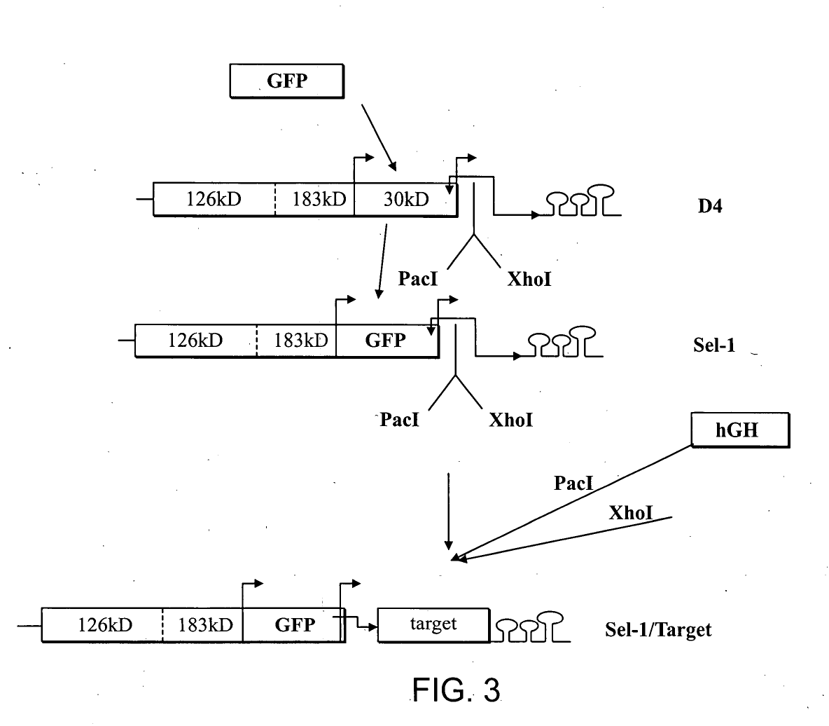

FIG. 3 presents a schematic diagram of the engineering of a TMV based viral construct containing a polynucleotide encoding growth hormone and a gene encoding a marker, e.g., a marker that allows for detection and/or selection. The upper portion of the figure shows the genomic organization of a TMV based virus construct, D4. The middle portion of the figure shows the same construct after insertion of a gene encoding a detectable marker (GFP) replacing the MP coding sequence. The lower portion of the figure shows the same construct following insertion of a polynucleotide encoding growth hormone (e.g., a gene encoding hGH, etc., indicated as “target”). The 126/183 kDa protein is required for replication of the virus. Arrows indicate positions of the subgenomic promoters. Transcription of the detectable marker is under control of the MP subgenomic promoter. Transcription of the inserted polynucleotide encoding growth hormone is under control of the TMV CP subgenomic promoter. However, other promoters could be used as described above. The 3′ portion of the construct includes TMV coat protein sequences that contain a cis element that may be required for optimal replication and that may form stem-loop structure(s) as shown.

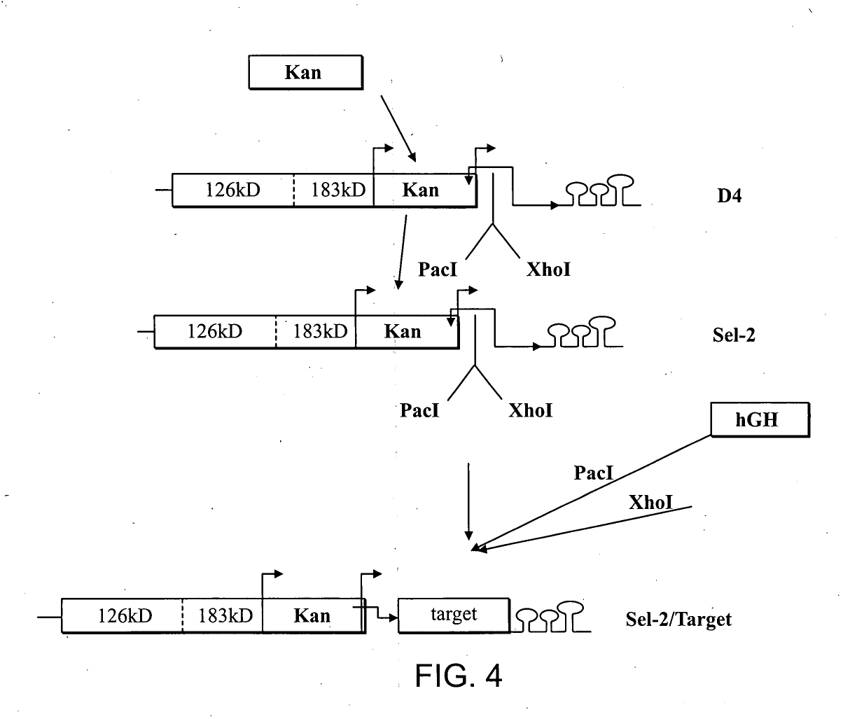

FIG. 4 shows a vector similar to that shown in FIG. 3 except that a selectable marker (a gene encoding a protein that confers resistance to kanamycin) is inserted instead of a gene encoding GFP. Including a gene that encodes a detectable or selectable marker in addition to a polynucleotide encoding growth hormone or a pharmaceutically active portion thereof is useful in the identification of clonal root lines and clonal plant cell lines that contain the vector and/or for identifying those lines that exhibit high and/or stable levels of expression.

In general, a wide variety of different markers can be used in accordance with the present invention. In general, a suitable marker for use in the invention is a detectable marker or a selectable marker. It is noted that in accordance with the practice in the art, the term “marker” can refer either to a nucleotide sequence, e.g., a gene, that encodes a product (protein) that allows for detection or selection, or can be used to refer to the protein itself. The term “selectable marker” is used herein as it is generally understood in the art and refers to a marker whose presence within a cell or organism confers a significant growth or survival advantage or disadvantage on the cell or organism under certain defined culture conditions (selective conditions). For example, the conditions may be the presence or absence of a particular compound or a particular environmental condition such as increased temperature, increased radiation, presence of a compound that is toxic in the absence of the marker, etc. The presence or absence of such compound(s) or environmental condition(s) is referred to as a “selective condition” or “selective conditions”. By “growth advantage” is meant either enhanced viability (e.g., cells or organisms with the growth advantage have an increased life span, on average, relative to otherwise identical cells), increased rate of proliferation (also referred to herein as “growth rate”) relative to otherwise identical cells or organisms, or both. In general, a population of cells having a growth advantage will exhibit fewer dead or nonviable cells and/or a greater rate of cell proliferation that a population of otherwise identical cells lacking the growth advantage. Although typically a selectable marker will confer a growth advantage on a cell, certain selectable markers confer a growth disadvantage on a cell, e.g., they make the cell more susceptible to the deleterious effects of certain compounds or environmental conditions than otherwise identical cells not expressing the marker.

Antibiotic resistance markers are a non-limiting example of a class of selectable marker that can be used to select cells that express the marker. In the presence of an appropriate concentration of antibiotic (selective conditions), such a marker confers a growth advantage on a cell that expresses the marker. Thus cells that express the antibiotic resistance marker are able to survive and/or proliferate in the presence of the antibiotic while cells that do not express the antibiotic resistance marker are not able to survive and/or are unable to proliferate in the presence of the antibiotic. For example, a selectable marker of this type that is commonly used in plant cells is the NPTII protein, which encodes a protein that provides resistance against the antibiotic kanamycin. Additional selectable markers include proteins that confer resistance against carbenecillin (e.g., β-lactamases), proteins that confer resistance against gentamicin, hygronycin, etc.)

A second non-limiting class of selectable markers are nutritional markers. Such markers are generally enzymes that function in a biosynthetic pathway to produce a compound that is needed for cell growth or survival. In general, under nonselective conditions the required compound is present in the environment or is produced by an alternative pathway in the cell. Under selective conditions, functioning of the biosynthetic pathway in which the marker is involved is needed to produce the compound.

In general, a detectable marker is a marker whose presence within a cell can be detected through means other than subjecting the cell to a selective condition or directly measuring the level of the marker itself. Thus in general, the expression of a detectable marker within a cell results in the production of a signal that can be detected and/or measured. The process of detection or measurement may involve the use of additional reagents and may involve processing of the cell. For example, where the detectable marker is an enzyme, detection or measurement of the marker will typically involve providing a substrate for the enzyme. Preferably the signal is a readily detectable signal such as light, fluorescence, luminescence, bioluminescence, chemiluminescence, enzymatic reaction products, stainable products, or color. Thus preferred detectable markers for use in the present invention include fluorescent proteins such as green fluorescent protein (GFP) and variants thereof. Other suitable markers include luciferase, yellow fluorescent protein (YFP), lichenase, β-galactosidase, alkaline phosphatase, etc. Preferably the detectable marker is one that can be detected in intact, living root and/or plant cells.

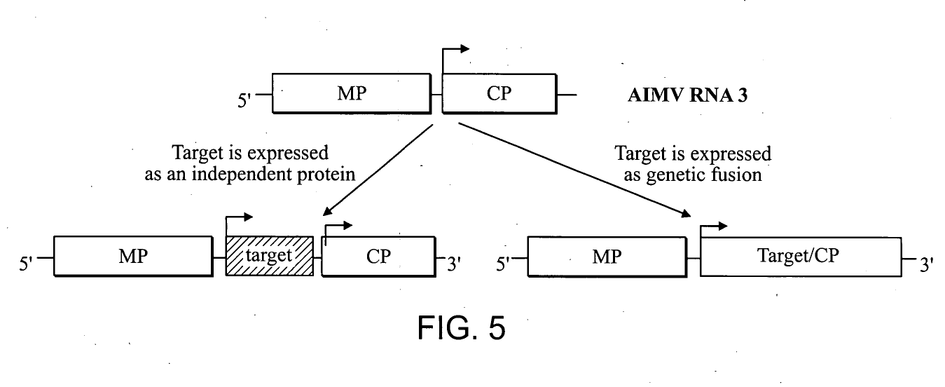

Another example of a viral vector for use in the present invention is an AIMV vector in which a polynucleotide encoding growth hormone is inserted, as shown in FIG. 5. For example, the polynucleotide encoding growth hormone or a pharmaceutically active portion thereof may replace the native AIMV CP encoding component in RNA3 of AIMV. Transcription of the polynucleotide encoding growth hormone or a pharmaceutically active portion thereof may be placed under control of the AIMV CP promoter. Alternately, the polynucleotide may replace the AIMV MP encoding component, and its transcription may be placed under control of the AIMV MP promoter. In other embodiments the inserted polynucleotide does not replace endogenous viral sequences. The polynucleotide encoding growth hormone or a pharmaceutically active portion thereof may be inserted in frame with CP coding sequences (complete or partial), so that a fusion protein is produced. In certain embodiments of the invention the fusion protein comprises a cleavage site between the CP portion and the remainder, so that the fusion protein can be cleaved to yield a growth hormone or a pharmaceutically active portion thereof protein free of CP sequences (or containing only a small number of such sequences). In certain embodiments of the invention the fusion protein assembles into particles, which can facilitate purification and/or antigen presentation (see, e.g., U.S. Pat. Nos. 6,042,832 and 6,448,070).

Yet another example of a vector useful in the practice of the present invention is a cauliflower mosaic virus (CMV) viral vector in which a polynucleotide encoding growth hormone or a pharmaceutically active portion thereof is inserted under control of the CMV CP promoter, replacing the CMV CP encoding component found in the genome of naturally occurring CMV.

In certain embodiments of the invention it is desirable to insert a portion of coding or noncoding sequence from a viral vector of one virus type into a viral vector of another type. For example, certain sequences may enhance replication or expression, etc. Such sequences may comprise, for example, part or all of a viral transcript 5′ or 3′ UTR.

Generally, in order to preserve viral function and also simply for ease of genetic manipulation, viral vectors will be prepared by altering an existing plant virus genome, for example by removing particular genes and/or by disrupting or substituting particular sequences so as to inactivate or replace them. In such circumstances, the vectors will show very high sequence identity with natural viral genomes. Of course, completely novel vectors may also be prepared, for example, by separately isolating individual desired genetic elements and linking them together, optionally with the inclusion of additional elements. It is noted that when a plant virus vector is said to affirmatively express a particular protein or activity needed for viral replication, movement, or some other viral function, it is not necessary that the relevant gene be identical to the corresponding gene found in nature. So long as the protein is functional, it may be used in accordance with the present invention. Very high sequence identity with the natural protein, however, is generally preferred. For instance, large deletions (e.g., greater than about 25 amino acids) should generally be avoided according to certain embodiments of the invention. Typically, viral proteins expressed in accordance with the present invention will show at least 50%, preferably 60%, 70%, 80%, 85%, 90%, 91%, 92%, 93%, 94%, 95%, 96%, 97%, 98%, or 99% sequence identity with the corresponding natural viral protein. More particularly, the inventive viral protein should typically show 100% identity with critical functional portions (typically of at least several amino acids, often of at least 10, 20, 30, 40, 50 or more amino acids) of the relevant natural viral protein.

It is noted that in the case of many proteins a number of amino acid changes can be made without significantly affecting the functional activity and/or various other properties of the protein such as stability, etc. In particular, many proteins tolerate conservative amino acid changes, i.e., the substitution of an amino acid with a different amino acid having similar properties (conservative substitution) at many positions without significant reduction in activity. Conservative amino acid substitution is well known in the art and represents one approach to obtaining a polypeptide having similar or substantially similar properties to those of a given polypeptide while altering the amino acid sequence. In general, amino acids have been classified and divided into groups according to (1) charge (positive, negative, or uncharged); (2) volume and polarity; (3) Grantham's physico-chemical distance; and combinations of these. See, e.g., Zhang, J., J. Mol. Evol., 50: 56-68, 2000; Grantham R., Science, 85: 862-864, 1974; Dagan, T., et al., Mol. Biol. Evol., 19(7), 1022-1025, 2002; Biochemistry, 4th Ed., Stryer, L., et al., W. Freeman and Co., 1995; and U.S. Pat. No. 6,015,692. For example, amino acids may be divided into the following 6 categories based on volume and polarity: special (C); neutral and small (A, G, P, S, T); polar and relatively small (N, D, Q, E), polar and relatively large (R, H, K), nonpolar and relatively small (I, L, M, V), and nonpolar and relatively large (F, W, Y). A conservative amino acid substitution may be defined as one that replaces one amino acid with an amino acid in the same group. Thus a variety of functionally equivalent proteins can be derived by making one or more amino acid substitutions, e.g., conservative amino acid substitutions, in a given viral protein.

D. Introducing Vectors Into Plants

In general, viral vectors may be delivered to plants according to known techniques. For example, the vectors themselves may be directly applied to plants (e.g., via abrasive inoculations, mechanized spray inoculations, vacuum infiltration, particle bombardment, or electroporation). Alternatively, virions may be prepared (e.g., from already infected plants), and may be applied to other plants according to known techniques.

As noted herein, in particular aspects of the present invention, viral vectors are applied to plants (e.g., plant, portion of plant, sprout, etc) (e.g., through infiltration or mechanical inoculation, spray, etc.). Where infection is to be accomplished by direct application of a viral genome to a plant, any available technique may be used to prepare the genome. For example, many viruses that are usefully employed in accordance with the present invention have ssRNA genomes. ssRNA may be prepared by transcription of a DNA copy of the genome, or by replication of an RNA copy, either in vivo or in vitro. Given the readily availability of easy-to-use in vitro transcription systems (e.g., SP6, T7, reticulocyte lysate, etc.), and also the convenience of maintaining a DNA copy of an RNA vector, it is expected that inventive ssRNA vectors will often be prepared by in vitro transcription, particularly with T7 or SP6 polymerase.

In certain embodiments of the invention rather than introducing a single viral vector type into the plant, multiple different viral vectors are introduced. Such vectors may, for example, trans-complement each other with respect to functions such as replication, cell-to-cell movement, and/or long distance movement. The vectors may contain different polynucleotides encoding growth hormone or a pharmaceutically active portion thereof, e.g., polynucleotides that encode individual polypeptides that associate to form a single protein complex such as antibodies, etc., or polynucleotides that encode different enzymes in a biosynthetic pathway. Selection for roots that express multiple polypeptides encoding growth hormone or a pharmaceutically active portion thereof may be performed as described above for single polynucleotides or polypeptides.

II. Clonal Plant and Plant Tissue Expression Systems

Methods and reagents for generating a variety of clonal entities derived from plants which are useful for the production of growth hormone (e.g., human growth hormone, a pharmaceutically active fragment thereof) are provided. Clonal entities include clonal root lines, clonal root cell lines, clonal plant cell lines, and clonal plants capable of production of growth hormone (e.g., human growth hormone, a pharmaceutically active fragment thereof). The invention further provides methods and reagents for expression of growth hormone polynucleotide and polypeptide products in clonal cell lines derived from various plant tissues (e.g., roots, leaves), and in whole plants derived from single cells (clonal plants). Such methods are typically based on the use of plant viral vectors of various types.

For example, in one aspect, the invention provides methods of obtaining a clonal root line that expresses a polynucleotide encoding growth hormone or a pharmaceutically active portion thereof comprising steps of: (i) introducing a viral vector that comprises a polynucleotide encoding growth hormone or a pharmaceutically active portion thereof into a plant or portion thereof; and (ii) generating one or more clonal root lines from the plant. The clonal root lines may be generated, for example, by infecting the plant or plant portion (e.g., a harvested piece of leaf) with an Agrobacterium (e.g., A. rhizogenes) that causes formation of hairy roots. Clonal root lines can be screened in various ways to identify lines that maintain the virus, lines that express the polynucleotide encoding growth hormone or a pharmaceutically active portion thereof at high levels, etc. The invention further provides clonal root lines, e.g., clonal root lines produced according to the inventive methods and further encompasses methods of expressing polynucleotides and producing polypeptides encoding growth hormone or a pharmaceutically active portion thereof using the clonal root lines.

The invention further provides methods of generating a clonal root cell line that expresses a polynucleotide encoding growth hormone or a pharmaceutically active portion thereof comprising steps of: (i) generating a clonal root line, cells of which contain a viral vector whose genome comprises a polynucleotide encoding growth hormone or a pharmaceutically active portion thereof; (ii) releasing individual cells from the clonal root line; and (iii) maintaining the cells under conditions suitable for root cell proliferation. The invention provides clonal root cell lines and methods of expressing polynucleotides and producing polypeptides using the clonal root cell lines.

In another aspect, the invention provides methods of generating a clonal plant cell line that expresses a polynucleotide encoding growth hormone or a pharmaceutically active portion thereof comprising steps of: (i) generating a clonal root line, cells of which contain a viral vector whose genome comprises a polynucleotide encoding growth hormone or a pharmaceutically active portion thereof; (ii) releasing individual cells from the clonal root line; and (iii) maintaining the cells in culture under conditions appropriate for plant cell proliferation. The invention further provides methods of generating a clonal plant cell line that expresses a polynucleotide encoding growth hormone or a pharmaceutically active portion thereof comprising steps of: (i) introducing a viral vector that comprises a polynucleotide encoding growth hormone or a pharmaceutically active portion thereof into cells of a plant cell line maintained in culture; and (ii) enriching for cells that contain the viral vector. Enrichment may be performed, for example, by (i) removing a portion of the cells from the culture; (ii) diluting the removed cells so as to reduce the cell concentration; (iii) allowing the diluted cells to proliferate; and (iv) screening for cells that contain the viral vector. Clonal plant cell lines may be used for production of a growth hormone polypeptide or a pharmaceutically active portion thereof in accordance with the present invention.

The invention includes a number of methods for generating clonal plants, cells of which contain a viral vector that comprises a polynucleotide encoding growth hormone or a pharmaceutically active portion thereof. For example, the invention provides methods of generating a clonal plant that expresses a polynucleotide encoding growth hormone or a pharmaceutically active portion thereof comprising steps of: (i) generating a clonal root line, cells of which contain a viral vector whose genome comprises a polynucleotide encoding growth hormone or a pharmaceutically active portion thereof; (ii) releasing individual cells from the clonal root line; and (iii) maintaining released cells under conditions appropriate for formation of a plant. The invention further provides methods of generating a clonal plant that expresses a polynucleotide encoding growth hormone or a pharmaceutically active portion thereof comprising steps of: (i) generating a clonal plant cell line, cells of which contain a viral vector whose genome comprises a polynucleotide encoding growth hormone or a pharmaceutically active portion thereof; and (ii) maintaining the cells under conditions appropriate for formation of a plant. In general, clonal plants according to the invention can express any polynucleotide encoding growth hormone or a pharmaceutically active portion thereof. Such clonal plants can be used for production of a growth hormone polypeptide or a pharmaceutically active portion thereof.

As noted above, the present invention provides systems for expressing a polynucleotide or polynucleotides encoding growth hormone or a pharmaceutically active portion thereof in clonal root lines, clonal root cell lines, clonal plant cell lines (e.g., cell lines derived from leaf, stem, etc.), and in clonal plants. The polynucleotide encoding growth hormone or a pharmaceutically active portion thereof is introduced into an ancestral plant cell using a plant viral vector whose genome includes the polynucleotide encoding growth hormone or a pharmaceutically active portion thereof operably linked to (i.e., under control of) a promoter. A clonal root line or clonal plant cell line is established from the cell containing the virus according to any of several techniques further described below. The plant virus vector or portions thereof can be introduced into the plant cell by infection, by inoculation with a viral transcript or infectious cDNA clone, by electroporation, by T-DNA mediated gene transfer, etc.

The following sections describe methods for generating clonal root lines, clonal root cell lines, clonal plant cell lines, and clonal plants that express a polynucleotide encoding growth hormone or a pharmaceutically active portion thereof are then described. A “root line” is distinguished from a “root cell line” in that a root line produces actual rootlike structures or roots while a root cell line consists of root cells that do not form rootlike structures. The use of the term “line” is intended to indicate that cells of the line can proliferate and pass genetic information on to progeny cells. Cells of a cell line typically proliferate in culture without being part of an organized structure such as those found in an intact plant. The use of the term “root line” is intended to indicate that cells in the root structure can proliferate without being part of a complete plant. It is noted that the term “plant cell” encompasses root cells. However, to distinguish the inventive methods for generating root lines and root cell lines from those used to directly generate plant cell lines from non-root tissue (as opposed to generating clonal plant cell lines from clonal root lines or clonal plants derived from clonal root lines), the terms “plant cell” and “plant cell line” as used herein generally refer to cells and cell lines that consist of non-root plant tissue. The plant cells can be, for example, leaf, stem, shoot, flower part, etc. It is noted that seeds can be derived from the clonal plants generated as derived herein. Such seeds will also contain the viral vector as will plants obtained from such seeds. Methods for obtaining seed stocks are well known in the art. See, e.g., U.S. Ser. No. 10/294,314.

A. Clonal Root Lines

The present invention provides methods for generating a clonal root line in which a plant viral vector is used to direct expression of a polynucleotide encoding growth hormone or a pharmaceutically active portion thereof. One or more viral expression vector(s) including a polynucleotide encoding growth hormone or a pharmaceutically active portion thereof operably linked to a promoter is introduced into a plant or a portion thereof according to any of a variety of known methods. For example, as described in Example 2, plant leaves can be inoculated with viral transcripts. The vectors themselves may be directly applied to plants (e.g., via abrasive inoculations, mechanized spray inoculations, vacuum infiltration, particle bombardment, or electroporation). Alternatively, virions may be prepared (e.g., from already infected plants), and may be applied to other plants according to known techniques.

Where infection is to be accomplished by direct application of a viral genome to a plant, any available technique may be used to prepare the genome. For example, many viruses that are usefully employed in accordance with the present invention have ssRNA genomes. ssRNA may be prepared by transcription of a DNA copy of the genome, or by replication of an RNA copy, either in vivo or in vitro. Given the readily availability of easy-to-use in vitro transcription systems (e.g., SP6, T7, reticulocyte lysate, etc.), and also the convenience of maintaining a DNA copy of an RNA vector, it is expected that inventive ssRNA vectors will often be prepared by in vitro transcription, particularly with T7 or SP6 polymerase. Infectious cDNA clones can also be used. Agrobacterially mediated gene transfer can also be used to transfer viral nucleic acids such as viral vectors (either entire viral genomes or portions thereof) to plant cells using, e.g., agroinfiltration, according to methods known in the art.

The plant or plant portion may then be then maintained (e.g., cultured or grown) under conditions suitable for replication of the viral transcript. In certain embodiments of the invention the virus spreads beyond the initially inoculated cell, e.g., locally from cell to cell and/or systemically from an initially inoculated leaf into additional leaves. However, in other embodiments of the invention the virus does not spread. Thus the viral vector may contain genes encoding functional MP and/or CP, but may be lacking one or both of such genes. In general, the viral vector is introduced into (infects) multiple cells in the plant or portion thereof.

Following introduction of the viral vector into the plant, leaves are harvested. In general, leaves may be harvested at any time following introduction of the viral vector. However, it may be preferable to maintain the plant for a period of time following introduction of the viral vector into the plant, e.g., a period of time sufficient for viral replication and, optionally, spread of the virus from the cells into which it was initially introduced. A clonal root culture (or multiple cultures) is prepared, e.g., by known methods further described below and in Example 2.

In general, any available method may be used to prepare a clonal root culture from a plant or plant tissue into which a viral vector has been introduced. One such method employs genes that exist in certain bacterial plasmids. These plasmids are found in various species of Agrobacterium that infect and transfer DNA to a wide variety of organisms. As a genus, Agrobacteria can transfer DNA to a large and diverse set of plant types including numerous dicot and monocot angiosperm species and gymnosperms (See, Gelvin, S. B., “Agrobacterium-Mediated Plant Transformation: the Biology behind the “Gene-Jockeying” Tool”, Microbiology and Molecular Biology Reviews, 67(1): 16-37 (2003) and references therein, all of which are incorporated herein by reference). The molecular basis of genetic transformation of plant cells is transfer from the bacterium and integration into the plant nuclear genome of a region of a large tumor-inducing (Ti) or rhizogenic (Ri) plasmid that resides within various Agrobacterial species. This region is referred to as the T-region when present in the plasmid and as T-DNA when excised from the plasmid. Generally, a single-stranded T-DNA molecule is transferred to the plant cell in naturally occurring Agrobacterial infection and is ultimately incorporated (in double-stranded form) into the genome. Systems based on Ti plasmids are widely used for introduction of foreign genetic material into plants and for production of transgenic plants.

Infection of plants with various Agrobacterial species and transfer of the T-DNA has a number of effects. For example, A. tumefaciens causes crown gall disease while A. rhizogenes causes development of hairy roots at the site of infection, a condition known as “hairy root disease”. Each root arises from a single genetically transformed cell. Thus root cells in the roots are clonal, and each root represents a clonal population of cells. The roots produced by A. rhizogenes infection are characterized by a high growth rate and genetic stability. (Giri, A. and Narasu, M. L., Biotechnology Advances, 18: 1-22 (2000) and references therein, all of which are incorporated herein by reference). In addition, such roots are able to regenerate genetically stable plants (Giri 2000).

In general, the present invention encompasses the use of any strain of Agrobacteria, particularly A. rhizogenes strains, that is capable of inducing formation of roots from plant cells. As mentioned above, a portion of the Ri plasmid (Ri T-DNA) is responsible for causing hairy root disease. While transfer of this portion of the Ri plasmid to plant cells can conveniently be accomplished by infection with Agrobacteria harboring the Ri plasmid, the invention also encompasses the use of alternative methods of introducing the relevant region into a plant cell. Such methods include any available method of introducing genetic material into plant cells including, but not limited to, biolistics, electroporation, PEG-mediated DNA uptake, Ti-based vectors, etc. The relevant portions of the Ri T-DNA can also be introduced into plant cells by use of a viral vector. The Ri genes can be included in the same vector that contains the polynucleotide encoding growth hormone or a pharmaceutically active portion thereof or in a different viral vector, which can be the same or a different type to that of the vector that contains the polynucleotide encoding growth hormone or a pharmaceutically active portion thereof. It is noted that the entire Ri T-DNA may not be required for production of hairy roots, and the invention encompasses the use of portions of the Ri T-DNA, provided that such portions contain sufficient genetic material to induce root formation, as known in the art. Additional genetic material, e.g., genes present within the Ri plasmid but not within the T-DNA, may also be transferred to the plant cell in accordance with the invention, particularly genes whose expression products facilitate integration of the T-DNA into the plant cell DNA.

In order to prepare a clonal root line in accordance with certain embodiments of the invention, the harvested leaf portions are contacted with A. rhizogenes under conditions suitable for infection and transformation. Example 2 describes one method for generating root lines from leaves into which a viral vector has been introduced. The leaf portions are maintained in culture to allow development of hairy roots. Each root is clonal, i.e., cells in the root are derived from a single ancestral cell into which the Ri T-DNA was transferred. In accordance with the invention, a portion of such ancestral cells will also contain the viral vector. Thus cells in a root derived from such an ancestral cell will also contain the viral vector since it will be replicated and will be transmitted during cell division. Thus a high proportion, preferably at least 50%, more preferably at least 75%, at least 80%, at least 90%, at least 95%, or all (100%) or substantially all (at least 98%) of the cells will contain the viral vector. It is noted that since the viral vector is inherited by daughter cells within the clonal root, movement of the viral vector within the root is not necessary to maintain the viral vector throughout the root. Individual clonal hairy roots may be removed from the leaf portion and further cultured. Such roots are also referred to herein as root lines. Isolated clonal roots continue to grow following isolation.

As described in Examples 2-4, a variety of different clonal root lines have been generated using the inventive methods. These root lines were generated using viral vectors containing polynucleotides encoding growth hormone or a pharmaceutically active portion thereof (e.g., encoding, hGH). The root lines were tested by Western blot. Root lines displayed a variety of different expression levels of the various polypeptides. Root lines displaying high expression were selected and further cultured. These root lines were subsequently tested again and shown to maintain high levels of expression over extended periods of time, indicating stability. The level of expression was comparable to or greater than expression in intact plants infected with the same viral vector used to generate the clonal root lines. In addition, the stability of expression of the root lines was superior to that obtained in plants infected with the same viral vector. Up to 80% of such virus-infected plants reverted to wild type after 2-3 passages. (Such passages involved inoculating plants with transcripts, allowing the infection (local or systemic) to become established, taking a leaf sample, and inoculating fresh plants that are subsequently tested for expression.)

The root lines may be cultured on a large scale for production of growth hormone or a pharmaceutically active portion thereof polypeptides as discussed further below. It is noted that the clonal root lines (and cell lines derived from the clonal root lines) can generally be maintained in medium that does not include various compounds, e.g., plant growth hormones such as auxins, cytokinins, etc., that are typically employed in the culture of root and plant cells. This feature greatly reduces the expense associated with tissue culture, and the inventors expect that it will contribute significantly to the economic feasibility of protein production using plants.

Any of a variety of methods may be used to select clonal roots that express a polynucleotide encoding growth hormone or a pharmaceutically active portion thereof. Western blots, ELISA assays, etc., can be used to detect an encoded polypeptide. In the case of detectable markers such as GFP, alternative methods such as visual screens can be performed. If a viral vector that contains a polynucleotide that encodes a selectable marker is used, an appropriate selection can be imposed (e.g., the leaf material and/or roots derived therefrom can be cultured in the presence of an appropriate antibiotic or nutritional condition and surviving roots identified and isolated). Certain viral vectors contain two or more polynucleotides encoding growth hormone or a pharmaceutically active portion thereof, e.g., two or more polynucleotides encoding different polypeptides. If one of these is a selectable or detectable marker, clonal roots that are selected or detected by selecting for or detecting expression of the marker will have a high probability of also expressing the second polynucleotide. Screening for root lines that contain particular polynucleotides can also be performed using PCR and other nucleic acid detection methods.

Alternatively, clonal root lines can also be screened for presence of the virus by inoculating host plants that will form local lesions as a result of virus infection (e.g., hypersensitive host plants). For example, 5 mg of root tissue can be homogenized in 50 ul of phosphate buffer and used to inoculate a single leaf of a tobacco plant. If the virus is present in root cultures, within two to three days characteristic lesions will appear on the infected leaves. This means that the root line contains recombinant virus that carries the polynucleotide encoding growth hormone or a pharmaceutically active portion thereof (target gene). If no local lesions are formed, there is no virus, and the root line is rejected as negative. This method is highly time and cost efficient. After initially screening for the presence of virus, roots that contain the virus are subjected to secondary screening, e.g., by Western blot or ELISA to select high expressers. Additional screens, e.g., screens for rapid growth, growth in particular media or under particular environmental conditions, etc., can also be applied. These screening methods may, in general, be applied in the development of any of the clonal root lines, clonal root cell lines, clonal plant cell lines, and/or clonal plants described herein.

As will be evident to one of ordinary skill in the art, a variety of modifications may be made to the description of the inventive methods for generating clonal root lines that contain a viral vector. Such modifications are within the scope of the invention. For example, while it is generally preferred to introduce the viral vector into an intact plant or portion thereof prior to introduction of the Ri T-DNA genes, in certain embodiments of the invention the Ri-DNA is introduced prior to introducing the viral vector. In addition, it is also possible to contact intact plants with A. rhizogenes rather than harvesting leaf portions and then exposing them to the bacterium.

Other methods of generating clonal root lines from single cells of the plant or portion thereof that harbor the viral vector can also be used (i.e., methods not using A. rhizogenes or genetic material from the Ri plasmid). For example, treatment with certain plant hormones or combinations of plant hormones is known to result in generation of roots from plant tissue.

B. Clonal Cell Lines Derived from Clonal Root Lines

As described above, the invention provides methods for generating clonal root lines, wherein cells in the root lines contain a viral vector. As is well known in the art, a variety of different cell lines can be generated from roots. For example, root cell lines can be generated from individual root cells obtained from the root using a variety of known methods. Such root cell lines may be obtained from various different root cell types within the root. In general, root material is harvested and dissociated (e.g., physically and/or enzymatically digested) to release individual root cells, which are then further cultured. Complete protoplast formation is generally not necessary. If desired, root cells can be plated at very dilute cell concentrations, so as to obtain root cell lines from single root cells. Root cell lines derived in this manner are clonal root cell lines contain the viral vector. Such root cell lines therefore exhibit stable expression of the polynucleotide encoding growth hormone or a pharmaceutically active portion thereof. Clonal plant cell lines can also be obtained in a similar manner from the clonal roots, e.g., by culturing dissociated root cells in the presence of the appropriate plant hormones. Screens and successive rounds of enrichment can be used to identify cell lines that express the polynucleotide encoding growth hormone or a pharmaceutically active portion thereof at high levels. However, if the clonal root line from which the cell line is derived already expresses at high levels, such additional screens may be unnecessary.

As in the case of the clonal root lines, cells of a clonal root cell line are derived from a single ancestral cell that contains the viral vector and will, therefore, also contain the viral vector since it will be replicated and will be transmitted during cell division. Thus a high proportion, preferably at least 50%, more preferably at least 75%, at least 80%, at least 90%, at least 95%, or all (100%) or substantially all (at least 98%) of the cells will contain the viral vector. It is noted that since the viral vector is inherited by daughter cells within the clonal root cell line, movement of the viral vector among the cells is not necessary to maintain the viral vector. The clonal root cell lines can be used for production of a polynucleotide encoding growth hormone or a pharmaceutically active portion thereof as described below.

C. Clonal Plant Cell Lines

The present invention provides methods for generating a clonal plant cell line in which a plant viral vector is used to direct expression of a polynucleotide encoding growth hormone or a pharmaceutically active portion thereof. According to the inventive method, one or more viral expression vector(s) including a polynucleotide encoding growth hormone or a pharmaceutically active portion thereof operably linked to a promoter is introduced into cells of a plant cell line that is maintained in cell culture. A number of plant cell lines from various plant types are known in the art, any of which can be used. Newly derived cell lines can also be generated according to known methods for use in practicing the invention. A viral vector is introduced into cells of the plant cell line according to any of a number of methods. For example, as described in Example 5, protoplasts can be made and viral transcripts then electroporated into the cells. Other methods of introducing a plant viral vector into cells of a plant cell line can also be used.

A method for generating clonal plant cell lines in accordance with the invention and a viral vector suitable for introduction into plant cells (e.g., protoplasts) can be used as follows: Following introduction of the viral vector, the plant cell line may be maintained in tissue culture. During this time the viral vector may replicate, and polynucleotides encoding growth hormone or a pharmaceutically active portion thereof may be expressed. Clonal plant cell lines are derived from the culture, e.g., by a process of successive enrichment. For example, samples may be removed from the culture, optionally with dilution so that the concentration of cells is low, and plated in Petri dishes in individual droplets. The droplets are then maintained to allow cell division.

It will be appreciated that the droplets may contain a variable number of cells, depending on the initial density of the culture and the amount of dilution. The cells can be diluted such that most droplets contain either 0 or 1 cell if it is desired to obtain clonal cell lines expressing the polynucleotide encoding growth hormone or a pharmaceutically active portion thereof after only a single round of enrichment. However, it can be more efficient to select a concentration such that multiple cells are present in each droplet and then screen the droplets to identify those that contain expressing cells. In general, any appropriate screening procedure can be employed. For example, selection or detection of a detectable marker such as GFP can be used. Western blots or ELISA assays can also be used. Individual droplets (100 ul) contain more than enough cells for performance of these assays. Multiple rounds of enrichment are performed to isolate successively higher expressing cell lines. Single clonal plant cell lines (i.e, populations derived from a single ancestral cell) can be generated by further limiting dilution using standard methods for single cell cloning. However, it is not necessary to isolate individual clonal lines. A population containing multiple clonal cell lines can also be used for expression of a polynucleotide encoding growth hormone or a pharmaceutically active portion thereof.

In general, certain considerations described above for generation of clonal root lines also apply to the generation of clonal plant cell lines. For example, a diversity of viral vectors containing one or more polynucleotides encoding growth hormone or a pharmaceutically active portion thereof can be used as can combinations of multiple different vectors. Similar screening methods can also be used. As in the case of the clonal root lines and clonal root cell lines, cells of a clonal plant cell line are derived from a single ancestral cell that contains the viral vector and will, therefore, also contain the viral vector since it will be replicated and will be transmitted during cell division. Thus a high proportion, preferably at least 50%, more preferably at least 75%, at least 80%, at least 90%, at least 95%, or all (100%) or substantially all (at least 98%) of the cells will contain the viral vector. It is noted that since the viral vector is inherited by daughter cells within the clonal plant cell line, movement of the viral vector among the cells is not necessary to maintain the viral vector. The clonal plant cell line can be used for production of a polypeptide encoding growth hormone or a pharmaceutically active portion thereof as described below.

D. Clonal Plants

Clonal plants can be generated from the clonal roots, clonal root cell lines, and/or clonal plant cell lines produced according to the various methods described above. Methods for the generation of plants from roots, root cell lines, and plant cell lines such as the clonal root lines, clonal root cell lines, and clonal plant cell lines described herein are well known in the art (See, e.g., Peres et al., Plant Cell, Tissue, and Organ Culture 65, 37-44, 2001 and standard reference works on plant molecular biology and biotechnology cited elsewhere herein. The invention therefore provides a method of generating a clonal plant comprising steps of (i) generating a clonal root line, clonal root cell line, or clonal plant cell line according to any of the inventive methods described above; and (ii) generating a whole plant from the clonal root line, clonal root cell line, or clonal plant. The clonal plants may be propagated and grown according to standard methods. Example 7 describes generation of a clonal plant from a clonal root line containing a viral vector that encodes human growth hormone.

As in the case of the clonal root lines, clonal root cell lines, and clonal plant cell lines, the cells of a clonal plant are derived from a single ancestral cell that contains the viral vector and will, therefore, also contain the viral vector since it will be replicated and will be transmitted during cell division. Thus a high proportion, preferably at least 50%, more preferably at least 75%, at least 80%, at least 90%, at least 95%, or all (100%) or substantially all (at least 98%) of the cells will contain the viral vector. It is noted that since the viral vector is inherited by daughter cells within the clonal plant, movement of the viral vector is not necessary to maintain the viral vector.

III. Sprouts and Sprouted Seedling Plant Expression Systems

The present invention provides systems and methods of producing pharmaceutical peptides and proteins in edible sprouted seedlings. The present invention further provides edible sprouted seedlings as a biomass containing a pharmaceutical peptide or protein. In certain aspects, the biomass is provided directly for consumption. In other aspects, the biomass is processed prior to consumption, for example, by homogenizing, crushing, drying, or extracting. In yet other aspects, the pharmaceutical protein is purified from the biomass and formulated into a pharmaceutical composition.

Additionally provided are methods for producing pharmaceutical proteins in sprouted seedlings that can be consumed or harvested live (e.g., sprouts, sprouted seedlings of the Brassica species). In certain aspects, the present invention involves growing a seed to an edible sprouted seedling in a contained, regulatable environment (e.g., indoors, in a container, etc.). The seed can be a genetically engineered seed that contains an expression cassette encoding a pharmaceutically active protein, which expression is driven by an exogenously inducible promoter. A variety of exogenously inducible promoters can be used that are inducible, for example, by light, heat, phytohormones, nutrients, etc.

In related embodiments, the present invention provides methods of producing pharmaceutically active proteins in sprouted seedlings by first generating a seed stock for the sprouted seedling by transforming plants with an expression cassette that encodes pharmaceutically active protein using an Agrobacterium transformation system, wherein expression of the pharmaceutical protein is driven by an inducible promoter. Transgenic seeds can be obtained from the transformed plant, grown in a contained, regulatable environment, and induced to express the pharmaceutical protein.

In other embodiments methods are provided that involves infecting sprouted seedlings with a viral expression cassette encoding a pharmaceutically active protein whose expression is driven by a constitutive (or inducible) promoter. The sprouted seedlings are grown for two to fourteen days in a contained, regulatable environment or at least until sufficient levels of the pharmaceutical protein have been obtained for consumption or harvesting.

The present invention further provides systems for producing pharmaceutically active proteins in sprouted seedlings that include a housing unit with climate control and a sprouted seedling containing an expression cassette that encodes a pharmaceutically active protein, wherein the pharmaceutically active protein is driven by a constitutive or inducible promoter. The inventive systems can provide unique advantages over the outdoor environment or greenhouse, which cannot be controlled. This enables the grower to precisely time the induction of expression of the pharmaceutical protein. It can also greatly reduce the cost of producing the pharmaceutical protein.

In certain aspects, genetically engineered seeds or embryos that contain a transgene encoding a pharmaceutically active growth hormone peptide or protein are grown to the sprouted seedling stage in a contained, regulatable environment. The contained, regulatable environment may be a housing unit or room in which the seeds can be grown indoors. All environmental factors of the contained, regulatable environment may be controlled. Since sprouts do not require light to grow, and lighting can be expensive, the genetically engineered seeds or embryos may be grown to the sprouted seedling stage indoors in the absence of light.

Other environmental factors that can be regulated in the contained, regulatable environment of the present invention include temperature, humidity, water, nutrients, gas (e.g., O₂ or CO₂ content or air circulation), chemicals (small molecules such as sugars and sugar derivatives or hormones such as such as the phytohormones gibberellic or absisic acid, etc.) and the like.

According to certain methods of the present invention, expression of the transgene encoding the pharmaceutical protein may be controlled by an exogenously inducible promoter. Exogenously inducible promoters are caused to increase or decrease expression of a transgene in response to an external, rather than an internal stimulus. A number of these environmental factors can act as inducers for expression of the transgenes carried by the expression cassettes of the genetically engineered sprouts. The promoter may be a heat-inducible promoter, such as a heat-shock promoter. For example, using as heat-shock promoter the temperature of the contained environment may simply be raised to induce expression of the transgene. Other promoters include light inducible promoters. Light-inducible promoters can be maintained as constitutive promoters if the light in the contained regulatable environment is always on. Alternatively, expression of the transgene can be turned on at a particular time during development by simply turning on the light. The promoter may be a chemically inducible promoter is used to induce expression of the transgene. According to these embodiments, the chemical could simply be misted or sprayed onto the seed, embryo, or seedling to induce expression of the transgene. Spraying and misting can be precisely controlled and directed onto the target seed, embryo, or seedling to which it is intended. The contained environment is devoid of wind or air currents, which could disperse the chemical away from the intended target, so that the chemical stays on the target for which it was intended.

According to the present invention, the time expression is induced can be selected to maximize expression of the pharmaceutical protein in the sprouted seedling by the time of harvest. Inducing expression in an embryo at a particular stage of growth, for example, inducing expression in an embryo at a particular number of days after germination, may result in maximum synthesis of the pharmaceutical protein at the time of harvest. For example, inducing expression from the promoter 4 days after germination may result in more protein synthesis than inducing expression from the promoter after 3 days or after 5 days. Those skilled in the art will appreciate that maximizing expression can be achieved by routine experimentation. In preferred methods, the sprouted seedlings are harvested at about 1, 2, 3, 4, 5, 6, 7, 8, 9, 10, 11, or 12 days after germination.

In cases where the expression vector has a constitutive promoter instead of an inducible promoter, the sprouted seedling may be harvested at a certain time after transformation of the sprouted seedling. For example, if a sprouted seedling were virally transformed at an early stage of development, for example, at the embryo stage, the sprouted seedlings may be harvested at a time when expression is at its maximum post-transformation, e.g., at about 1, 2, 3, 4, 5, 6, 7, 8, 9, 10, 11, 12, 13, or 14 days post-transformation. It could also be that sprouts develop one, two, three or more months post-transformation, depending on the germination of the seed.

Generally, once expression of the pharmaceutical protein begins, the seeds, embryos, or sprouted seedlings are allowed to grow until sufficient levels of the pharmaceutical protein are expressed. In certain aspects, sufficient levels are levels that would provide a therapeutic benefit to a patient if the harvested biomass were eaten raw. Alternatively, sufficient levels are levels from which the pharmaceutical protein can be concentrated or purified from the biomass and formulated into a pharmaceutical composition that provides a therapeutic benefit to a patient upon administration. Typically, the pharmaceutical protein is not a protein expressed in the sprouted seedling in nature. At any rate, the pharmaceutical protein is preferably expressed at concentrations above that which would be present in the sprouted seedling in nature.

Once expression of the pharmaceutical protein is induced, growth is allowed to continue until the sprouted seedling stage, at which time the sprouted seedlings are harvested. The sprouted seedlings can be harvested live. Harvesting live sprouted seedlings has several advantages including minimal effort and breakage. The sprouted seedlings of the present invention may be preferably grown hydroponically, making harvesting a simple matter of lifting the sprouted seedling from its hydroponic solution. No soil is required for the growth of the sprouted seedlings of the invention, but may be provided if deemed necessary or desirable by the skilled artisan. Because sprouts can be grown without soil, no cleansing of sprouted seedling material is required at the time of harvest. Being able to harvest the sprouted seedling directly from its hydroponic environment without washing or scrubbing minimizes breakage of the harvested material. Breakage and wilting of plants induces apoptosis. During apoptosis, certain proteolytic enzymes become active, which can degrade the pharmaceutical protein expressed in the sprouted seedling, resulting in decreased therapeutic activity of the protein. Apoptosis-induced proteolysis significantly decreases the yield of protein from mature plants. Using the methods of the present invention, apoptosis is avoided because no harvesting takes place until the moment the proteins are extracted from the plant.

For example, live sprouts may be ground, crushed, or blended to produce a slurry of sprouted seedling biomass, in a buffer containing protease inhibitors. The buffer may be maintained at about 4° C. In other aspects, the sprouted seedling biomass is air-dried, spray dried, frozen, or freeze-dried. As in mature plants, some of these methods, such as air-drying, may result in a loss of activity of the pharmaceutical protein. However, because sprouted seedlings are very small and have a large surface area to volume ratio, this is much less likely to occur. Those skilled in the art will appreciate that many techniques for harvesting the biomass that minimize proteolysis of the pharmaceutical protein are available and could be applied to the present invention.

In some embodiments, the sprouted seedlings are edible. In certain embodiments, sprouted seedlings expressing sufficient levels of pharmaceutical proteins are consumed upon harvesting (e.g., immediately after harvest, within minimal period following harvest) so that absolutely no processing occurs before the sprouted seedlings are consumed. In this way, any harvest-induced proteolytic breakdown of the pharmaceutical protein before administration of the pharmaceutical protein to a patient in need of treatment is minimized. For example, sprouted seedlings that are ready to be consumed can be delivered directly to a patient. Alternatively, genetically engineered seeds or embryos are delivered to a patient in need of treatment and grown to the sprouted seedling stage by the patient. In one aspect, a supply of genetically engineered sprouted seedlings are provided to a patient, or to a doctor who will be treating patients, so that a continual stock of sprouted seedlings expressing certain desirable pharmaceutical proteins may be cultivated. This may be particularly valuable for populations in developing countries, where expensive pharmaceuticals are not affordable or deliverable. The ease with which the sprouted seedlings of the invention can be grown makes the sprouted seedlings of the present invention particularly desirable for such developing populations.

The regulatable nature of the contained environment imparts advantages to the present invention over growing plants in the outdoor environment. In general, growing genetically engineered sprouted seedlings that express pharmaceutical proteins in plants provides a pharmaceutical product faster (because the plants are harvested younger) and with less effort, risk, and regulatory considerations than growing genetically engineered plants. The contained, regulatable environment used in the present invention reduces or eliminates the risk of cross-pollinating plants in the nature.

For example, a heat inducible promoter could never be used in the outdoors because the outdoor temperature cannot be controlled. The promoter would be turned on any time the outdoor temperature rose above a certain level. Similarly, the promoter would be turned off every time the outdoor temperature dropped. Such temperature shifts could occur in a single day, for example, turning expression on in the daytime and off at night. A heat inducible promoter, such as those described herein, would not even be practical for use in a greenhouse, which is susceptible to climatic shifts to almost the same degree as the outdoors. Growth of genetically engineered plants in a greenhouse is quite costly. In contrast, in the present system, every variable can be controlled so that the maximum amount of expression can be achieved with every harvest.

In certain aspects, the sprouted seedlings of the present invention are grown in trays that can be watered, sprayed, or misted at any time during the development of the sprouted seedling. For example, the tray may be fitted with one or more watering, spraying, misting, and draining apparatus that can deliver and/or remove water, nutrients, chemicals etc. at specific time and at precise quantities during development of the sprouted seedling. For example, seeds require sufficient moisture to keep them damp. Excess moisture drains through holes in the trays into drains in the floor of the room. Preferably, drainage water is treated as appropriate for removal of harmful chemicals before discharge back into the environment.

Another advantage of the trays of the present system is that they can be contained within a very small space. Since no light is required for the sprouted seedlings to grow, the trays containing seeds, embryos, or sprouted seedlings may be tightly stacked vertically on top of one another, providing a large quantity of biomass per unit floor space in a housing facility constructed specifically for these purposes. In addition, the stacks of trays can be arranged in horizontal rows within the housing unit. Once the seedlings have grown to a stage appropriate for harvest (about two to fourteen days) the individual seedling trays are moved into a processing facility, either manually or by automatic means, such as a conveyor belt.

The system of the present invention is unique in that it provides a sprouted seedling biomass, which is a source of a pharmaceutically active protein. Whether consumed directly or processed into the form of a pharmaceutical composition, because the sprouted seedlings are grown in a contained, regulatable environment, the sprouted seedling biomass and/or pharmaceutical composition derived from the biomass can be provided to a consumer at low cost. In addition, the fact that the conditions for growth of the sprouted seedlings can be controlled makes the quality and purity of the product consistent. The contained, regulatable environment of the invention also obviates many safety regulations of the EPA that can prevent scientists from growing genetically engineered agricultural products out of doors.

A. Generating Transformed Sprouts

A variety of methods can be used to transform plant cells and produce genetically engineered sprouted seedlings. Two available methods for the transformation of plants that require that transgenic plant cell lines be generated in vitro, followed by regeneration of the cell lines into whole plants include Agrobacterium tumefaciens mediated gene transfer and microprojectile bombardment or electroporation. Viral transformation is a more rapid and less costly methods of transforming embryos and sprouted seedlings that can be harvested without an experimental or generational lag prior to obtaining the desired product. For any of these techniques, the skilled artisan would appreciate how to adjust and optimize transformation protocols that have traditionally been used for plants, seeds, embryos, or spouted seedlings.

Agrobacterium Transformation Expression Cassettes. Agrobacterium is a representative genus of the gram-negative family Rhizobiaceae. This species is responsible for plant tumors such as crown gall and hairy root disease. In dedifferentiated plant tissue, which is characteristic of tumors, amino acid derivatives known as opines are produced by the Agrobacterium and catabolized by the plant. The bacterial genes responsible for expression of opines are a convenient source of control elements for chimeric expression cassettes. According to the present invention, the Agrobacterium transformation system may be used to generate edible sprouted seedlings, which are merely harvested earlier than the mature plants. Agrobacterium transformation methods can easily be applied to regenerate sprouted seedlings expressing pharmaceutical proteins.

In general, transforming plants involves the transformation of plant cells grown in tissue culture by co-cultivation with an Agrobacterium tumefaciens carrying a plant/bacterial vector. The vector contains a gene encoding a pharmaceutical protein. The Agrobacterium transfers the vector to the plant host cell and is then eliminated using antibiotic treatment. Transformed plant cells expressing the pharmaceutical protein are selected, differentiated, and finally regenerated into complete plantlets (Hellens et al., Plant Molecular Biology (2000) 42(819-832); Pilon-Smits et al, Plant Physiolog. (January 1999) 119(1):123-132; Barfield and Pua Plant Cell Reports (1991)10(6/7):308-314); Riva et al., Journal of Biotechnology (Dec. 15, 1998) 1(3), each incorporated by reference herein.

Expression vectors for use in the present invention include a gene (or expression cassette) encoding a pharmaceutical protein designed for operation in plants, with companion sequences upstream and downstream of the expression cassette. The companion sequences are generally of plasmid or viral origin and provide necessary characteristics to the vector to transfer DNA from bacteria to the desired plant host.

The basic bacterial/plant vector construct preferably provides a broad host range prokaryote replication origin, a prokaryote selectable marker. Suitable prokaryotic selectable markers include resistance toward antibiotics such as ampicillin or tetracycline. Other DNA sequences encoding additional functions that are well known in the art may also be present in the vector.

Agrobacterium T-DNA sequences are required for Agrobacterium mediated transfer of DNA to the plant chromosome. The tumor-inducing genes of the T-DNA are typically removed and replaced with sequences encoding the pharmaceutical protein. The T-DNA border sequences are retained because they initiate integration of the T-DNA region into the plant genome. If expression of the pharmaceutical protein is not readily amenable to detection, the bacterial/plant vector construct will also include a selectable marker gene suitable for determining if a plant cell has been transformed, e.g., the nptII kanamycin resistance gene. On the same or different bacterial/plant vector (Ti plasmid) are Ti sequences. Ti sequences include the virulence genes, which encode a set of proteins responsible for the excision, transfer and integration of the T-DNA into the plant genome (Schell, Science (1987) 237:1176-1183). Other sequences suitable for permitting integration of the heterologous sequence into the plant genome may also include transposon sequences, and the like, for homologous recombination.

Certain constructs will include the expression cassette encoding the growth hormone protein. One, two, or more expression cassettes may be used in a given transformation. The recombinant expression cassette contains, in addition to the pharmaceutical protein encoding sequence, at least the following elements: a promoter region, plant 5′ untranslated sequences, initiation codon (depending upon whether or not the expressed gene has its own), and transcription and translation termination sequences. In addition, transcription and translation terminators may be included in the expression cassettes or chimeric genes of the present invention. Signal secretion sequences that allow processing and translocation of the protein, as appropriate, may also be included in the expression cassette. A variety of promoters, signal sequences, and transcription and translation terminators are described, for example, in Lawton et al., Plant Mol. Biol (1987) 9:315-324 or U.S. Pat. No. 5,888,789, incorporated herein by reference. In addition, structural genes for antibiotic resistance are commonly utilized as a selection factor (Fraley et al. Proc. Natl. Acad. Sci., USA (1983) 80:4803-4807), incorporated herein by reference. Unique restriction enzyme sites at the 5′ and 3′ ends of the cassette allow for easy insertion into a pre-existing vector. Other binary vector systems for Agrobacterium-mediated transformation, carrying at least one T-DNA border sequence are described in PCT/EP99/07414, incorporated herein by reference.

Regeneration. Seeds of transformed plants are harvested, dried, cleaned, and tested for viability and for the presence and expression of a desired gene product. Once this has been determined, seed stock is stored under appropriate conditions of temperature, humidity, sanitation, and security to be used when necessary. Whole plants are then regenerated from cultured protoplasts, e.g., as described in Evans et al., Handbook of Plant Cell Cultures, Vol. 1: MacMillan Publishing Co. New York, 1983); and Vasil I. R. (ed.), Cell Culture and Somatic Cell Genetics of Plants, Acad. Press, Orlando, Vol. 1, 1984, and Vol. III, 1986, incorporated herein by reference. In certain aspects, the plants are regenerated only to the sprouted seedling stage. In other aspects, whole plants are regenerated to produce seed stocks and sprouted seedlings are generated from the seeds of the seed stock.

All plants from which protoplasts can be isolated and cultured to give whole, regenerated plants can be transformed by the present invention so that whole plants are recovered that contain the transferred gene. It is known that practically all plants can be regenerated from cultured cells or tissues, including, but not limited to, all major species of plants that produce edible sprouts. Some suitable plants include alfalfa, mung bean, radish, wheat, mustard, spinach, carrot, beet, onion, garlic, celery, rhubarb, a leafy plant such as cabbage or lettuce, watercress or cress, herbs such as parsley, mint, or clovers, cauliflower, broccoli, soybean, lentils, edible flowers such as the sunflower etc.

Means for regeneration vary from one species of plants to the next. However, those skilled in the art will appreciate that generally a suspension of transformed protoplants containing copies of the heterologous gene is first provided. Callus tissue is formed and shoots may be induced from callus and subsequently rooted. Alternatively, embryo formation can be induced from the protoplast suspension. These embryos germinate as natural embryos to form plants. Steeping the seed in water or spraying the seed with water to increase the moisture content of the seed to between 35-45% initiates germination. For germination to proceed, the seeds are typically maintained in air saturated with water under controlled temperature and airflow conditions. The culture media will generally contain various amino acids and hormones, such as auxin and cytokinins. It is also advantageous to add glutamic acid and proline to the medium, especially for such species as alfalfa. Shoots and roots normally develop simultaneously. Efficient regeneration will depend on the medium, the genotype, and the history of the culture. If these three variables are controlled, then regeneration is fully reproducible and repeatable.

The mature plants, grown from the transformed plant cells, are selfed and non-segregating, homozygous transgenic plants are identified. The inbred plant produces seeds containing the chimeric gene of the present invention. These seeds can be germinated and grown to the sprouted seedling stage to produce the pharmaceutical growth hormone protein or polypeptide or a pharmaceutically active fragment thereof.

In related embodiments, the seeds of the present invention are formed into seed products and sold with instructions on how to grow the seedlings to the appropriate sprouted seedling stage for administration or harvesting into a pharmaceutical composition. In other related embodiments, hybrids or novel varieties embodying the desired traits are developed from the inbred plants of the invention.

Direct Integration. Direct integration of DNA fragments into the genome of plant cells by microprojectile bombardment or electroporation may also be used in the present invention (see, e.g., Kikkert, J. R. Humiston et al., In Vitro Cellular & Developmental Biology. Plant: Journal of the Tissue Culture Association. (Jan/February 1999) 35 (1):43-50; Bates, G. W. Florida State University, Tallahassee, Fla. Molecular Biotechnology (October 1994) 2(2):135-145). More particularly, vectors containing a chimeric gene of the present invention can be introduced into plant cells by a variety of techniques. As described above, the vectors may include selectable markers for use in plant cells. The vectors may also include sequences that allow their selection and propagation in a secondary host, such as sequences containing an origin of replication and selectable marker. Typically, secondary hosts include bacteria and yeast. In one preferred embodiment, the secondary host is bacteria (e.g., Escherichia coli, the origin of replication is a colE 1-type origin of replication) and the selectable marker is a gene encoding ampicillin resistance. Such sequences are well known in the art and are commercially available (e.g., Clontech, Palo Alto, Calif. or Stratagene, La Jolla, Calif.).

The vectors of the present invention may also be modified to intermediate plant transformation plasmids that contain a region of homology to an Agrobacterium tumefaciens vector, a T-DNA border region from Agrobacterium tumefaciens, and chimeric genes or expression cassettes described above. Further vectors may include a disarmed plant tumor inducing plasmid of Agrobacterium tumefaciens.

According to the present embodiment, direct transformation of the vectors invention involves microinjecting the vectors directly into plant cells by the use of micropipettes to mechanically transfer the recombinant DNA (see, e.g., Crossway, Mol. Gen. Genet., 202:179-185, 1985, incorporated herein by reference). The genetic material may also be transferred into the plant cell by using polyethylene glycols (see, e.g., Krens et al., Nature (1982) 296:72-74). Another method of introducing nucleic acid segments is high velocity ballistic penetration by small particles with the nucleic acid either within the matrix of small beads or particles, or on the surface (see, e.g., Klein et al., Nature (1987) 327:70-73; Knudsen and Muller Planta (1991) 185:330-336)). Yet another method of introduction is fusion of protoplasts with other entities, either minicells, cells, lysosomes, or other fusible lipid-surfaced bodies (see, e.g., Fraley et al., Proc. Natl. Acad. Sci. USA (1982) 79:1859-1863). Vectors of the invention may also be introduced into plant cells by electroporation (see, e.g., Fromm et al. Proc. Natl. Acad. Sci. USA (1985) 82:5824). According to this technique, plant protoplasts are electroporated in the presence of plasmids containing the gene construct. Electrical impulses of high field strength reversibly permeabilize biomembranes allowing the introduction of the pasmids. Electroporated plant protoplasts reform the cell wall divide and form plant callus, which can be regenerated to form the sprouted seedlings of the invention. Those skilled in the art would appreciate how to utilize these methods to transform plants cells that can be used to generate edible sprouted seedlings.

Viral Transformation. Expression and inexpensive recovery of peptides with adequate biological activity is important for different applications including development of subunit vaccines. Some applications, however, require full-length, biologically active proteins. Similarly to conventional expression systems, plant virus vectors can also be used to produce full-length proteins, including growth hormone. According to the present invention, plant virus vectors are used to infect and produce growth hormone protein in seeds, embryos, sprouted seedlings. Expression of high levels of foreign genes encoding short peptides as well as large complex proteins by tobamoviral vectors is described, for example, by McCormick et al. (Proc. Natl. Acad. Sci. USA (1999) 96:703-708; Kumagai et al. (Gene (2000) 245:169-174 and Verch et al. (J. Immunol. Methods (1998) 220, 69-75, each incorporated herein by reference). Thus, plant virus vectors have a demonstrated ability to express short peptides as well as large complex proteins.

In certain embodiments, transgenic sprouts, which express pharmaceutically active growth hormone, are generated utilizing a host/virus system. Transgenic sprouts produced by viral infection provide a source of transgenic protein that has already been demonstrated to be safe. For example, sprouts are free of contamination with animal pathogens. Unlike, for example, tobacco, proteins from an edible sprout could at least in theory be used in oral applications without purification, thus significantly reducing costs. In addition, a virus/sprout system also offers a much simpler, less expensive route for scale-up and manufacturing, since the trangenes are introduced into the virus, which can be grown up to a commercial scale within a few days. In contrast, transgenic plants can require up to 5-7 years before sufficient seeds or plant material are available for large-scale trials or commercialization.

According to the present invention, plant RNA viruses have certain advantages, which make them attractive as vectors for foreign protein expression. The molecular biology and pathology of a number of plant RNA viruses are well characterized and there is considerable knowledge of virus biology, genetics, and regulatory sequences. Most plant RNA viruses have small genomes and infectious cDNA clones are available to facilitate genetic manipulation. Once the infectious virus material enters the susceptible host cell, it replicates to high levels and spreads rapidly throughout the entire sprouted seedling (one to ten days post inoculation). Virus particles are easily and economically recovered from infected sprouted seedling tissue. Viruses have a wide host range, enabling the use of a single construct for infection of several susceptible species. These characteristics are easily transferable to sprouts.

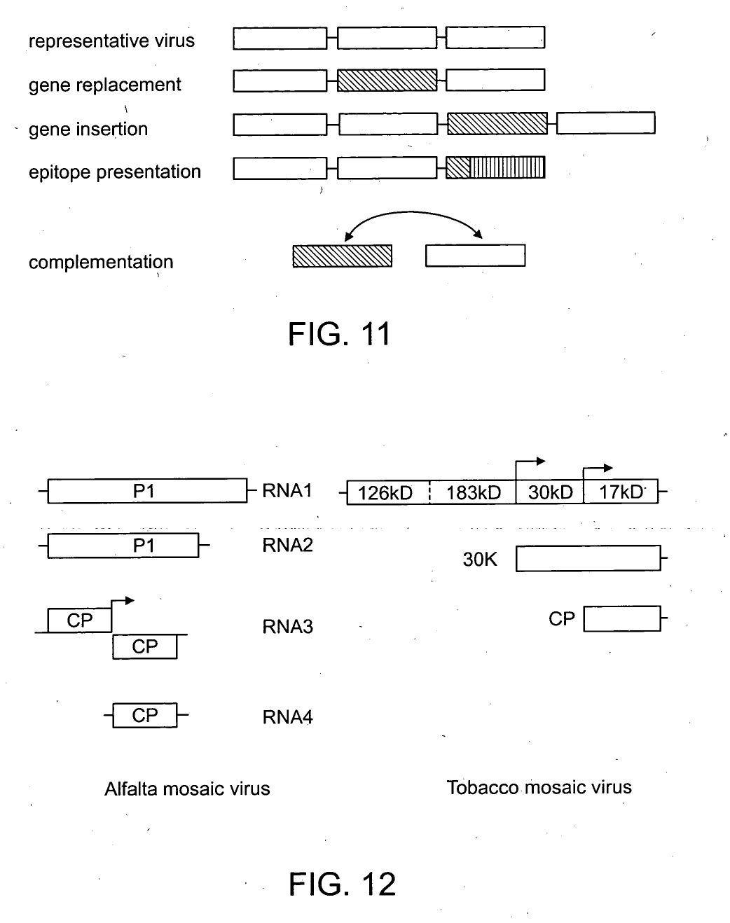

FIG. 11 illustrates several different strategies for expressing foreign genes using plant viruses. Foreign sequences can be expressed by replacing one of the viral genes with desired sequence, by inserting foreign sequences into the virus genome at an appropriate position, or by fusing foreign peptides to the structural proteins of a virus. Moreover, any of these approaches can be combined to express foreign sequences by trans-complementation of vital functions of a virus. A number of different strategies exist as tools to express foreign sequences in virus-infected plants using tobacco mosaic virus (TMV), alfalfa mosaic virus (AIMV), and chimeras thereof.

The genome of AIMV is a representative of the Bromoviridae family of viruses and consists of three genomic RNAs (RNAs1-3) and subgenomic RNA (RNA4) (FIG. 12). Genomic RNAs1 and 2 encode virus replicase proteins P1 and 2, respectively. Genomic RNA3 encodes the cell-to-cell movement protein P3 and the coat protein (CP). The CP is translated from subgenomic RNA4, which is synthesized from genomic RNA3, and is required to start the infection. Studies have demonstrated the involvement of the CP in multiple functions, including genome activation, replication, RNA stability, symptom formation, and RNA encapsidation (see e.g., Bol et al., Virology (1971) 46: 73-85; Van Der Vossen et al., Virology (1994) 202: 891-903; Yusibov et al., Virology 208: 405-407; Yusibov et al., Virology (1998) 242: 1-5; Bol et al., (Review, 100 refs.). J. Gen. Virol. (1999) 80: 1089-1102; De Graaff, Virology (1995) 208: 583-589; Jaspars et al., Adv. Virus Res (1974). 19, 37-149; Loesch-Fries, Virology (1985)146: 177-187; Neeleman et al., Virology (1991) 181: 687-693; Neeleman et al., Virology (1993) 196: 883-887; Van Der Kuyl et al., Virology (1991) 183: 731-738; Van Der Kuyl et al., Virology (1991) 185: 496-499).

Encapsidation of viral particles is typically required for long distance movement of virus from inoculated to un-inoculated parts of the seed, embryo, or sprouted seedling and for systemic infection. According to the present invention, inoculation can occur at any stage of plant development. In embryos and sprouts, spread of the inoculated virus should be very rapid. Virions of AIMV are encapsidated by a unique CP (24 kD), forming more than one type of particle. The size (30- to 60-nm in length and 18 nm in diameter) and shape (spherical, ellipsoidal, or bacilliform) of the particle depends on the size of the encapsidated RNA. Upon assembly, the N-terminus of the AIMV CP is thought to be located on the surface of the virus particles and does not appear to interfere with virus assembly (Bol et al., Virology (1971) δ: 73-85). Additionally, the AIMV CP with an additional 38-amino acid peptide at its N-terminus forms particles in vitro and retains biological activity (Yusibov et al., J. Gen. Virol. (1995) 77: 567-573).

AIMV has a wide host range, which includes a number of agriculturally valuable crop plants, including plant seeds, embryos, and sprouts. Together, these characteristics make the AIMV CP an excellent candidate as a carrier molecule and AIMV an attractive candidate vector for the expression of foreign sequences in the plant at the sprout stage of development. Moreover, upon expression from a heterologous vector such as TMV, the AIMV CP encapsidates TMV genome without interfering with virus infectivity (Yusibov et al., Proc. Natl. Acad. Sci. USA (1997) 94: 5784-5788, incorporated herein by reference). This allows the use of TMV as a carrier virus for AIMV CP fused to foreign sequences.

TMV, the prototype of the tobamoviruses, has a genome consisting of a single plus-sense RNA encapsidated with a 17.0 kD CP, which results in rod-shaped particles (300 nm in length) (FIG. 12). The CP is the only structural protein of TMV and is required for encapsidation and long distance movement of the virus in an infected host (Saito et al., Virology (1990) 176: 329-336). The 183 and 126 kD proteins are translated from genomic RNA and are required for virus replication (Ishikawa et al., Nucleic Acids Res. (1986) 14: 8291-8308). The 30 kD protein is the cell-to-cell movement protein of virus (Meshi et al., EMBO J. (1987) δ: 2557-2563). Movement and coat proteins are translated from subgenomic mRNAs (Hunter et al., Nature (1976) 260: 759-760; Bruening et al., Virology (1976) 71: 498-517; Beachy et al., Virology (1976) 73: 498-507, each incorporated herein by reference).

Schematic representation of AIMV and TMV genomes are shown in FIG. 12. RNAs1 and 2 of AIMV encode replicase proteins P1 and P2, respectively; genomic RNA3 encodes cell-to-cell movement protein P3 and the viral coat protein (CP). The CP is translated from subgenomic RNA4 synthesized from genomic RNA3. The 126 kD and 183 kD proteins of TMV are required for replication; the 30 kD protein is the viral cell-to-cell movement protein; and the 17 kD protein is the CP of virus. The CP and the 30 kD protein are translated from subgenomic RNAs. Arrows indicate position of subgenomic promoters.

Other methods of transforming plant tissues include transforming the flower of the plant. Transformation of Arabidopsis thaliana can be achieved by dipping the plant flowers into a solution of Agrobacterium tumefaciens (Curtis and Nam, Transgenic Research (August 2001) 10 4:363-371; Qing et al., Molecular Breeding: New Strategies in Plant Improvement (February 2000). (1):67-72). Transformed plants are formed in the population of seeds generated by the “dipped” plants. At a specific point during flower development, a pore exists in the ovary wall through which Agrobacterium tumefaciens gains access to the interior of the ovary. Once inside the ovary, the Agrobacterium tumefaciens proliferates and transforms individual ovules (Desfeux et al., Plant Physiology (July 2000) 123(3):895-904). The transformed ovules follow the typical pathway of seed formation within the ovary.

IV. Plant Species

Any plant susceptible to viral infection may be utilized in accordance with the present invention. In general, it will often be desirable to utilize plants that are amenable to growth under defined conditions, for example in a greenhouse and/or in aqueous systems. It may also be desirable to select plants that are not typically consumed by human beings or domesticated animals and/or are not typically part of the human food chain, so that they may be grown outside without concern that the expressed polynucleotide may be undesirably ingested. In other embodiments, however, it will be desirable to employ edible plants.

Often, certain desirable plant characteristics will be determined by the particular polynucleotide to be expressed. To give but a few examples, when the polynucleotide encodes a protein to be produced in high yield (as will often be the case, for example, when therapeutic proteins are to be expressed), it will often be desirable to select plants with relatively high biomass (e.g., tobacco, which has the additional advantages that it is highly susceptible to viral infection, has a short growth period, and is not in the human food chain). Where the polynucleotide encodes a protein whose full activity requires (or is inhibited by) a particular post-translational modification, the ability (or inability) of certain plant species to accomplish the relevant modification (e.g., a particular glycosylation) may direct selection.

In certain preferred embodiments of the invention, crop plants, or crop-related plants are utilized. In some particularly preferred embodiments, edible plants are utilized.

Plants for use in accordance with the present invention include Angiosperms, Bryophytes (e.g., Hepaticae, Musci, etc.), Pteridophytes (e.g., ferns, horsetails, lycopods), Gymnosperms (e.g., conifers, cycase, Ginko, Gnetales), and Algae (e.g., Chlorophyceae, Phaeophyceae, Rhodophyceae, Myxophyceae, Xanthophyceae, and Euglenophyceae). Preferred are members of the family Leguminosae (Fabaceae; e.g., pea, alfalfa, soybean); Gramineae (Poaceae; e.g., corn, wheat, rice); Solanaceae, particularly of the genus Lycopersicon (e.g., tomato), Solanum (e.g., potato, eggplant), Capsium (e.e., pepper), or Nicotiana (e.g., tobacco); Umbelliferae, particularly of the genus Daucus (e.g., carrot), Apium (e.g., celery), or Rutaceae (e.g., oranges); Compositae, particularly of the genus Lactuca (e.g., lettuce); Brassicaceae (Cruciferae), particularly of the genus Brassica or Sinapis. In certain aspects, preferred plants of the invention may be plants of the Brassica or Arabidopsis species. Some preferred Brassicaceae family members include Brassica campestris, B. carinata, B. juncea, B. napus, B. nigra, B. oleraceae, B. tournifortii, Sinapis alba, and Raphanus sativus. Some suitable plants that are amendable to transformation and are edible as sprouted seedlings include alfalfa, mung bean, radish, wheat, mustard, spinach, carrot, beet, onion, garlic, celery, rhubarb, a leafy plant such as cabbage or lettuce, watercress or cress, herbs such as parsley, mint, or clovers, cauliflower, broccoli, soybean, lentils, edible flowers such as the sunflower etc.

V. Polynucleotides and Polypeptides Encoding Growth Hormone

The teachings of the present invention may be employed to deliver to and/or express in plant cells any polynucleotide encoding growth hormone protein. A polynucleotide encoding growth hormone may comprise naturally occurring nucleic acid sequence of growth hormone (e.g., human growth hormone). Additionally, in certain embodiments, polynucleotide sequence(s) may be modified to optimize expression of growth hormone polypeptide in plant systems. Encoded growth hormone proteins may be naturally-occurring growth hormone proteins, or may be designed or engineered proteins. For example, encoded protein may be full length growth hormone (e.g., human growth hormone) which consists of the naturally occurring growth hormone sequence. Growth hormone proteins also may be a protein fragment of full length growth hormone which retains functional activity of full length growth hormone. Furthermore, growth hormone proteins of use in the present invention include a modified amino acid sequence of full length growth hormone, which is at least 85%, at least 90%, at least 95%, at least 99% or more identical to the naturally occurring growth hormone protein sequence, and wherein the variant protein retains functional activity of full length, pharmaceutically active growth hormone.

Growth hormone proteins also include, for instance fusion proteins (e.g., fusion proteins incorporating part or all of a plant virus protein such as MP or CP). See, e.g., U.S. Pat. Nos. 6,448,070 and 6,660,500. Numerous types of fusion proteins may be encoded. A heterologous sequence may be fused to the 5′ or 3′ end of a plant virus protein or located internally. Numerous sequences of diverse origin may be included within a single fusion protein. The encoded protein may comprise a cleavage site, which may be encoded by the inserted polynucleotide or by the viral vector. See, e.g., U.S. Pat. No. 6,740,740. For example, the vector may comprise a portion that encodes a cleavage site upstream of a portion that encodes CP so that when a polynucleotide encoding growth hormone or a pharmaceutically active portion thereof is inserted between the CP promoter and the portion that encodes a cleavage site, the resulting open reading frame encodes a fusion protein containing a portion encoded by the polynucleotide encoding growth hormone or a pharmaceutically active portion thereof, a cleavage site, and part or all of the CP. Cleavage of the fusion protein at the cleavage site releases the encoded growth hormone or a pharmaceutically active portion thereof polypeptide. The cleavage site may be a site for cleavage by chemical means (e.g., cyanogen bromide) or by enzymatic means (e.g., by a protease such as trypsin, chymotrypsin, thrombin, pepsin, Staphylococcus aureus V8 protease, and Factor Xa protease).

In certain embodiments of the invention the polynucleotide encoding growth hormone or a pharmaceutically active portion thereof comprises a portion encoding a tag, e.g., a 6X-His tag, HA tag, Myc tag, FLAG tag, etc. Such tags may simplify the detection, isolation and/or purification of the protein. In certain embodiments of the invention the tag is a cleavable tag, e.g., a tag cleavable by chemical means or by enzymatic means as described above. Including a cleavage site allows the tag to be readily be removed from the translated polypeptide, e.g., after purification, resulting in a protein with wild type sequence. It is to be understood that the tag and/or cleavage site may be present within a viral vector into which a particular polynucleotide encoding growth hormone or a pharmaceutically active portion thereof is to be inserted and need not be present within the inserted polynucleotide itself. Once the polynucleotide is inserted, the entire portion comprising the region(s) that encode the tag, cleavage site, and newly inserted polynucleotide is considered a polynucleotide encoding growth hormone or a pharmaceutically active portion thereof.

In some instances, it may be desirable to utilize the inventive system to express more than one polypeptide chain in the same plant ccell or tissue (e.g., clonal root, clonal plant cell, clonal plant cell line, clonal plant, sprout, sprouted seedling, orther plant cell, plant tissue, plant) for example, using two different viral vectors each of which directs expression of a polynucleotide, inserting two different polynucleotides into one viral vector, utilizing a transgenic plant that expresses one or more polynucleotides to generate a clonal root, clonal plant cell, clonal plant cell line, clonal plant, etc. Such a strategy may be particularly useful, for example in order to produce a multimeric protein or to simultaneously produce two different proteins such as a growth hormone protein and a detectable or selectable marker.

The inventive system may be employed to infect, and/or to express a polynucleotide in plants at any stage of development including, for example, mature plants, seedlings, sprouts, and seeds. The system may be employed to infect any part of a plant (e.g., roots, leaves, stems, etc.) In certain aspects of the invention, the system is used to infect sprouts. Generally, a plant is considered to be a sprout when it is a seedling that does not require external nutrients or energy in the form of light or heat beyond what is required to achieve normal germination temperatures. Often, a seedling that is less than two weeks old, preferably less than 10 days old, is considered to be a sprout.

VI. Culturing or Growing Plants, Plant Cells and Plant Tissues

In general, standard methods known in the art may be used for culturing or growing the plants, plant cells, and/or plant tissues of the invention (e.g., clonal plants, clonal plant cells, clonal roots, clonal root lines, sprouts, sprouted seedlings, plants, etc.). A wide variety of culture media and bioreactors have been employed to culture hairy root cells, root cell lines, and plant cells. See, for example, Giri, A. and Narasu, M. L., Biotechnol. Adv. 18:1-22, 2000; Rao, S.R. and Ravishankar, G. A., Biotechnol. Adv. 20:101-153, 2002, and references in both of the foregoing, all of which are incorporated herein by reference. Clonal plants may be grown in any suitable manner.

VII. Pharmaceuticals

The pharmaceutical proteins of the present invention which are expressed in plants, plant cells, and/or plant tissues (e.g., sprouts, sprouted seedlings, roots, root culture, clonal cells, clonal cell lines, clonal plants, etc.), include any pharmaceutically active growth hormone protein or peptide, either prokaryotic or eukaryotic. Generally, the pharmaceutically active growth hormone proteins of interest include full length growth hormone (e.g., human grown hormone), a pharmaceutically active portion thereof, or a pharmaceutically active variant thereof.

The present invention also provides pharmaceutical proteins for veterinary use, such as growth hormone protein or a pharmaceutically active portion thereof which is active in veterinary use, which may be produced by the plant(s) or portion thereof (e.g., root, cell, sprout, cell line, plant, etc.) of the invention.

VIII. Isolation of Protein Products, Administration and Pharmaceutical Compositions

The present invention provides plants, plant cells, and plant tissues expressing a pharmaceutically active protein that maintains its pharmaceutical activity when administered to a subject in need thereof. Preferred subjects include vertebrates, preferably mammals, more preferably human. According to the present invention, the subjects include veterinary subjects such as bovines, ovines, canines, felines, etc. In certain aspects, the edible sprout is administered orally to a subject in a therapeutically effective amount. In other aspects, the pharmaceutically active protein is provided in a pharmaceutical preparation, as described herein.

According to the present invention, treatment of a subject with a pharmaceucially active growth hormone is intended to elicit a physiological effect. A pharmaceutically active protein may have healing curative or palliative properties against a disorder or disease and can be administered to ameliorate relieve, alleviate, delay onset of, reverse or lessen symptoms or severity of a disease or disorder. A pharmaceutically active growth hormone also may have prophylactic properties and can be used to prevent or delay the onset of a disease or to lessen the severity of such disease, disorder, or pathological condition when it does emerge. A physiological effect elicited by treatement of a subject with growth hormone according to the present invention can include an effect selected from the group consisting of an increase in lean body mass, an increase in bone density, an increase in bone growth, an increase in energy level, and an improved quality of life

The pharmaceutical preparations of the present invention can be administered in a wide variety of ways to the subject, such as, for example, orally enterally, nasally, parenterally, intramuscularly or intravenously, rectally, vaginally, topically, ocularly, pulmonarily, or by contact application. In a preferred aspect, a pharmaceutical protein expressed in a transgenic sprout is administered to a subject orally. In other aspects a pharmaceutically active protein expressed in a transgenic sprout is extracted and/or purified, and used for the preparation of a pharmaceutical composition. Proteins are isolated and purified in accordance with conventional conditions and techniques known in the art. These include methods such as extraction, precipitation, chromatography, affinity chromatography, electrophoresis, and the like.

In many embodiments of the present invention, it will be desirable to isolate polynucleotide expression products from the plant tissue(s), e.g., roots, root cells, plants, plant cells, that express them. It may also be desirable to formulate such isolated products for their intended use (e.g., as a pharmaceutical or diagnostic agent, or as a reagent, etc.). In other embodiments, it will be desirable to formulate the products together with some or all of the plant tissues that express them.

Where it is desirable to isolate the expression product from some or all of the plant cells or tissues that express it, any available purification techniques may be employed. Those of ordinary skill in the art are familiar with a wide range of fractionation and separation procedures (see, for example, Scopes et al., Protein Purification. Principles and Practice, 3^{rd} Ed., Janson et al., 1993; Protein Purification: Principles, High Resolution Methods, and Applications, Wiley-VCH, 1998; Springer-Verlag, NY, 1993; Roe, Protein Purification Techniques, Oxford University Press, 2001, each of which is incorporated herein by reference). Often, it will be desirable to render the product more than about 50%, preferably more than about 60%, 70%, 80%, 85%, 90%, 91%, 92%, 93%, 94%, 95%, 96%, 97%, 98%, or 99% pure. See, e.g., U.S. Pat. Nos. 6,740,740 and 6,841,659 for discussion of certain methods useful for purifying substances from plant tissues or fluids.

Where it is desirable to formulate the product together with the plant material, it will often be desirable to have utilized a plant that is not toxic to the relevant recipient (e.g., a human or other animal). Relevant plant tissue (e.g., cells, roots, leaves) may simply be harvested and processed according to techniques known in the art, with due consideration to maintaining activity of the expressed product. In certain embodiments of the invention, it is desirable to have expressed the polynucleotide in an edible plant (and, specifically in edible portions of the plant) so that the material can subsequently be eaten. For instance, where the polynucleotide encodes a nutritionally relevant protein, or a therapeutic protein that is active after oral delivery (when properly formulated), it may be desirable to produce the protein in an edible plant portion, and to formulate the expressed polynucleotide for oral delivery together with the some or all of the plant material with which the polynucleotide was expressed.

Where the polynucleotide encodes or produces a therapeutic agent, it may be formulated according to known techniques. For example, an effective amount of a pharmaceutically active product can be formulated together with one or more organic or inorganic, liquid or solid, pharmaceutically suitable carrier materials. A pharmaceutically active product produced according to the present invention may be employed in dosage forms such as tablets, capsules, troches, dispersions, suspensions, solutions, gelcaps, pills, caplets, creams, ointments, aerosols, powder packets, liquid solutions, solvents, diluents, surface active agents, isotonic agents, thickening or emulsifying agents, preservatives, and solid bindings, as long as the biological activity of the protein is not destroyed by such dosage form.

In general, the compositions may comprise any of a variety of different pharmaceutically acceptable carrier(s), adjuvant(s), or vehicle(s), or a combination of one or more such carrier(s), adjuvant(s), or vehicle(s). As used herein the language “pharmaceutically acceptable carrier, adjuvant, or vehicle” includes solvents, dispersion media, coatings, antibacterial and antifungal agents, isotonic and absorption delaying agents, and the like, compatible with pharmaceutical administration. Materials that can serve as pharmaceutically acceptable carriers include, but are not limited to sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening agents, flavoring agents, and perfuming agents, preservatives, and antioxidants can also be present in the composition, according to the judgment of the formulator (see also Remington's Pharmaceutical Sciences, Fifteenth Edition, E.W. martin (Mack Publishing Co., Easton Pa., 1975). For example, the polynucleotide expression product may be provided as a pharmaceutical composition by means of conventional mixing granulating dragee-making, dissolving, lyophilizing, or similar processes.

In certain situations, it may be desirable to prolong the effect of a pharmaceutical preparation by slowing the absorption of the pharmaceutically active product (e.g., protein) that is subcutaneously or intramuscularly injected. This may be accomplished by the use of a liquid suspension of crystalline or amorphous material with poor water solubility. The rate of absorption of the product then depends upon its rate of dissolution, which in turn, may depend upon size and form. Alternatively, delayed absorption of a parenterally administered product is accomplished by dissolving or suspending the product in an oil vehicle. Injectable depot forms are made by forming microcapsule matrices of the protein in biodegradable polymers such as polylactide-polyglycolide. Depending upon the ratio of product to polymer and the nature of the particular polymer employed, the rate of release can be controlled. Examples of other biodegradable polymers include poly(orthoesters) and poly(anhydrides). Depot injectable formulations may be prepared by entrapping the product in liposomes or microemulsions, which are compatible with body tissues. Alternative polymeric delivery vehicles can be used for oral formulations. For example, biodegradable, biocompatible polymers such as ethylene vinyl acetate, polyanhydrides, polyglycolic acid, collagen, polyorthoesters, and polylactic acid, etc., can be used. Growth hormone or a pharmaceutically active portion thereof may be formulated as microparticles, e.g., in combination with a polymeric delivery vehicle.

Enterally administered preparations of pharmaceutically active products may be introduced in solid, semi-solid, suspension or emulsion form and may be compounded with any pharmaceutically acceptable carriers, such as water, suspending agents, and emulsifying agents. The expression products may also be administered by means of pumps or sustained-release forms, especially when administered as a preventive measure, so as to prevent the development of disease in a subject or to ameliorate or delay an already established disease. Supplementary active compounds, e.g., compounds independently active against the disease or clinical condition to be treated, or compounds that enhance activity of an inventive compound, can also be incorporated into the compositions. Flavorants and coloring agents can also be used.

Pharmaceutically active products, optionally together with plant tissue, are particularly well suited for oral administration as pharmaceutical compositions. Oral liquid formulations can also be used and may be of particular utility for pediatric populations. Harvested plant material may be processed in any of a variety of ways (e.g., air drying, freeze drying, extraction etc.), depending on the properties of the desired therapeutic product and its desired form. Such compositions as described above are ingested orally alone or ingested together with food or feed or a beverage. Compositions for oral administration include plants; extractions of the plants, and proteins purified from infected plants provided as dry powders, foodstuffs, aqueous or non-aqueous solvents, suspensions, or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medial parenteral vehicles including sodium chloride solution, Ringer's dextrose solution, dextrose plus sodium chloride solution, Ringer's solution containing lactose or fixed oils. Examples of dry powders include any plant biomass that has been dried, for example, freeze dried, air dried, or spray dried. For example, the plants may be air dried by placing them in a commercial air dryer at about 120 degrees Fahrenheit until the biomass contains less than 5% moisture by weight. The dried plants may be stored for further processing as bulk solids or further processed by grinding to a desired mesh sized powder. Alternatively, freeze-drying may be used for products that are sensitive to air-drying. Products may be freeze dried by placing them into a vacuum drier and dried frozen under a vacuum until the biomass contains less than about 5% moisture by weight. The dried material can be further processed as described herein.

Plant-derived material may be administered as or together with one or more herbal preparations. Useful herbal preparations include liquid and solid herbal preparations. Some examples of herbal preparations include tinctures, extracts (e.g., aqueous extracts, alcohol extracts), decoctions, dried preparations (e.g., air-dried, spray dried, frozen, or freeze-dried), powders (e.g., lyophilized powder), and liquid. Herbal preparations can be provided in any standard delivery vehicle, such as a capsule, tablet, suppository, liquid dosage, etc. Those skilled in the art will appreciate the various formulations and modalities of delivery of herbal preparations that may be applied to the present invention.

Those skilled in the art will also appreciate that a method of obtaining the desired pharmaceutically active products is by extraction. Plant material (e.g., roots, leaves, etc.) may be extracted to remove the desired products from the residual biomass, thereby increasing the concentration and purity of the product. Plants may also be extracted in a buffered solution. For example, the plant material may be transferred into an amount of ice-cold water at a ratio of one to one by weight that has been buffered with, e.g., phosphate buffer. Protease inhibitors can also be added as required. The plant material can be disrupted by vigorous blending or grinding while suspended in the buffer solution and the extracted biomass removed by filtration or centrifugation. The product carried in solution can be further purified by additional steps or converted to a dry powder by freeze-drying or precipitation. Extraction can also be carried out by pressing. Plants or roots can also be extracted by pressing in a press or by being crushed as they are passed through closely spaced rollers. The fluids expressed from the crushed plants or roots are collected and processed according to methods well known in the art. Extraction by pressing allows the release of the products in a more concentrated form. However, the overall yield of the product may be lower than if the product were extracted in solution.

Inventive root lines, cell lines, plants, extractions, powders, dried preparations and purified protein or nucleic acid products, etc., can also be in encapsulated form with or without one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active product may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

In other methods, a plant or portion thereof expressing a pharmaceutically active product according to the present invention, or biomass thereof, is administered orally as medicinal food. Such edible compositions are consumed by eating raw, if in a solid form, or by drinking, if in liquid form. The plant material can be directly ingested without a prior processing step or after minimal culinary preparation. For example, the pharmaceutically active protein is expressed in a sprout of which can be eaten directly. For example, the polynucleotide is expressed in an alfalfa sprout, mung bean sprout, or spinach or lettuce leaf sprout, etc. In an alternative embodiment, the plant biomass is processed and the material recovered after the processing step is ingested.

Processing methods used in the present invention are methods commonly used in the food or feed industry. The final products of such methods still include a substantial amount of the expressed pharmaceutically active polynucleotide or polypeptide and can be conveniently eaten or drunk. The final product may also be mixed with other food or feed forms, such as salts, carriers, favor enhancers, antibiotics, and the like, and consumed in solid, semi-solid, suspension, emulsion, or liquid form. Such methods can include a conservation step, such as, e.g., pasteurization, cooking, or addition of conservation and preservation agents. Any plant is used and processed in the present invention to produce edible or drinkable plant matter. The amount of pharmaceutically active polynucleotide or polypeptide expression product in a plant-derived preparation may be tested by methods standard in the art, e.g., gel electrophoresis, ELISA, or Western blot analysis, using a probe or antibody specific for the product. This determination may be used to standardize the amount of polynucleotide or protein ingested. For example, the amount of therapeutically active product may be determined and regulated, for example, by mixing batches of product having different levels of product so that the quantity of material to be drunk or eaten to ingest a single dose can be standardized. The contained, regulatable environment of the present invention, however, should minimize the need to carry out such standardization procedures.

A pharmaceutically active polynucleotide or protein produced in a plant cell or tissue and eaten by a subject may be preferably absorbed by the digestive system. One advantage of the ingestion of plant tissue that has been only minimally processed is to provide encapsulation or sequestration of the polynucleotide or protein in cells of the plant. Thus, the product may receive at least some protection from digestion in the upper digestive tract before reaching the gut or intestine and a higher proportion of active product would be available for uptake.

The pharmaceutical compositions of the present invention can be administered therapeutically or prophylactically. The compositions may be used to treat or prevent a disease. For example, any individual who suffers from a disease or who is at risk of developing a disease may be treated. It will be appreciated that an individual can be considered at risk for developing a disease without having been diagnosed with any symptoms of the disease. For example, if the individual has a particular genetic marker identified as being associated with increased risk for developing a particular disease, that individual will be considered at risk for developing the disease. Similarly, if members of an individual's family have been diagnosed with a particular disease, e.g., cancer, the individual may be considered to be at risk for developing that disease.

Liquid dosage forms for oral administration include, but are not limited to, pharmaceutically acceptable emulsions, microemulsions, solutions, suspensions, syrups, and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Compositions for rectal or vaginal administration are preferably suppositories or retention enemas, which can be prepared by mixing the compositions of this invention with suitable non-irritating excipients or carriers such as cocoa butter, polyethylene glycol or a suppository wax which are solid at ambient temperature but liquid at body temperature and therefore melt in the rectum or vaginal cavity and release the active protein.

Dosage forms for topical, transmucosal or transdermal administration of a pharmaceutical composition of this invention include ointments, pastes, creams, lotions, gels, powders, solutions, sprays, inhalants or patches. The active product, or preparation thereof, is admixed under sterile conditions with a pharmaceutically acceptable carrier and any needed preservatives or buffers as may be required. For transmucosal or transdermal administration, penetrants appropriate to the barrier to be permeated may be used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, growth hormone or a pharmaceutically active portion thereof may also be formulated into ointments, salves, gels, or creams as generally known in the art. Ophthalmic formulation, eardrops, and eye drops are also contemplated as being within the scope of this invention. Additionally, the present invention contemplates the use of transdermal patches, which have the added advantage of providing controlled delivery of a pharmaceutically active protein to the body. Such dosage forms can be made by suspending or dispensing the pharmaceutically active product in the proper medium. Absorption enhancers can also be used to increase the flux of the pharmaceutically active protein across the skin. The rate can be controlled by either providing a rate controlling membrane or by dispersing the pharmaceutically active protein in a polymer matrix or gel.

The compositions are administered in such amounts and for such time as is necessary to achieve the desired result. As described above, in certain embodiments of the present invention a “therapeutically effective amount” of a pharmaceutical composition is that amount effective for treating, attenuating, or preventing a disease in a subject. Thus, the “amount effective to treat, attenuate, or prevent disease”, as used herein, refers to a nontoxic but sufficient amount of the pharmaceutical composition to treat, attenuate, or prevent disease in any subject. As but one example, the “therapeutically effective amount” can be an amount to treat, attenuate, or prevent growth hormone deficiency, etc.

The exact amount required will vary from subject to subject, depending on the species, age, and general condition of the subject, the stage of the disease, the particular pharmaceutical mixture, its mode of administration, and the like. The infected plants of the invention and/or protein preparations thereof are preferably formulated in dosage unit form for ease of administration and uniformity of dosage. The expression “dosage unit form,” as used herein, refers to a physically discrete unit of pharmaceutically active polynucleotide or polypeptide expression product appropriate for the patient to be treated. It will be understood, however, that the total daily usage of the compositions of the present invention is preferably decided by an attending physician within the scope of sound medical judgment. The specific therapeutically effective dose level for any particular patient or organism may depend upon a variety of factors including the disorder being treated and the severity of the disorder; the activity of the specific compound employed; the specific composition employed; the age, body weight, general health, sex of the patient, diet of the patient, pharmacokinetic condition of the patient, the time of administration, route of administration, and rate of excretion of the specific compound employed; the duration of the treatment; drugs used in combination or coincidental with the specific compound employed; and like factors well known in the medical arts.

It will also be appreciated that the pharmaceutical compositions of the present invention can be employed in combination therapies, that is, the pharmaceutical compositions can be administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another growth hormone activating agent), or they may achieve different effects.

IX. Kits

In still another aspect, the present invention also provides a pharmaceutical pack or kit including the live sprouted seedlings, clonal entity or plant producing growth hormone or a pharmaceutically active portion thereof of the present invention, or preparations, extracts, or pharmaceutical compositions containing the pharmaceutically active protein expressed by the sprouted seedlings, clonal entity or plant in one or more containers filled with one or more of the ingredients of the pharmaceutical compositions of the invention. In certain embodiments, the pharmaceutical pack or kit includes an additional approved therapeutic agent for use as a combination therapy. Optionally associated with such container(s) can be a notice in the form prescribed by a governmental agency regulating the manufacture, use or sale of pharmaceutical products, which notice reflects approval by the agency of manufacture, use, or sale for human administration.

The present invention involves the purification and affordable scaling up of the production of pharmaceutical proteins using any of a variety of plant expression systems known in the art and provided herein, including viral plant expression systems described herein. Kits are provided that include therapeutic reagents. As but one non-limiting example, the growth hormone, associated with the hypopituitarism and other disorders related to growth hormone deficiency, can be provided as oral formulations and administered as therapy. Alternatively, therapeutic growth hormone protein can be provided in an injectable formulation for administration. Pharmaceutical doses or instructions therefor are provided in the kit for administration to an individual diagnosed with a disease, e.g., a growth hormone deficient individual, or an individual at risk for developing a disease, e.g., hypopituitarism.

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. The following examples contain information, exemplification and guidance, which can be adapted to the practice of this invention in its various embodiments and the equivalents thereof.

### EXEMPLIFICATION

### Example 1

### Production of Pharmaceuticals in Sprouts by Agrobacterium Transformation

Seeds of plants are transformed by Agrobacterium are harvested, dried, cleaned, and tested for viability and presence of desired genetic material. Seed stock is stored under appropriate conditions until use. At the time of use, appropriate amounts of seeds are soaked in water containing an amount of surface sterilizing agent (e.g., Clorox® bleach) for 20 minutes to 4 hours. Seeds are spread onto a flat of trays, which contain provisions for sustenance of growth and drainage of water. Trays containing the seeds are put on racks in the contained, regulatable environment under controlled temperature, lighting, access, air circulation, water supply, and drainage. Trays are misted with water from misters equipped with automatic timers for one to 30 minutes at intervals of 30 minutes to four hours, sufficient to keep seeds damp. Excess moisture drains through holes in the trays into drains in the floor of the room.

Seeds are allowed to germinate and develop under controlled conditions. Seeds are incubated for about two to fourteen days before harvest and processing. At some point during the incubation process, from four hours to seven days prior to harvest, seeds are exposed to environmental conditions that cause the induction of an introduced or indigenous DNA promoter sequence that causes an increase in the synthesis of one or more desired proteins in the tissues of the sprouting seedling. A transient increase in the incubation temperature from about 30° C. to about 37° C. to cause induction of a heat shock promoter.

After incubation of the seedlings for two to fourteen days, the seedlings are harvested by moving the individual trays into a processing facility on a conveyor belt. The harvested seedlings are processed by extraction in phosphate buffered solution containing protease inhibitors. The seedlings are disrupted by vigorous blending or grinding while suspended in the buffer solution and the extracted biomass removed by centrifugation.

### Example 2

### Sprouts of Seedlings Transiently Infected by a Plants Virus

Seeds of desired plants are obtained from a contract of commercial grower as wild-type seeds. The seed stock is stored under appropriate conditions of temperature, humidity, sanitation, and security until use. At the time of use, appropriate amounts of seeds are soaked in water and incubated on trays as described above under controlled conditions.

After incubation for two to fourteen days, the germinated seedlings are sprayed with a solution containing a transgenic virus, and further or simultaneously treated with a material that causes mechanical abrasion of the plant leaf tissue. In this example, the leaves are abraded with a spray of air containing abrasive particles. The virus is allowed to systemically infect the plants for an appropriate period; from about one to about ten days and expression of the desired transgenic protein is monitored.

After infection of the seedlings for one to ten days, the seedlings are harvested as described in Example 1 above.

### Example 3

### Expression of Human Growth Hormone in Plants

Testing the stability and movement. To conduct viral stability and movement tests, small quantities of each construct are synthesized. Each construct contains a T7 or SP6 RNA polymerase promoter fused to the exact 5′ terminus of viral genomic RNA and a unique restriction site at the 3′ end that is used to linearize the plasmid prior to in vitro transcription. The T7 or SP6 RNA polymerase then generates run-off transcripts, which are used to inoculate plants. Plants are inoculated mechanically at two-leaf stage, by gently rubbing the inoculum onto the leaf surface in the presence of an abrasive agent, such as carborundum powder (320-grit; Fisher, Pittsburgh, Pa.). Five to ten plants are inoculated per construct and 1-2 μg of each RNA transcript is used per inoculation. The plants are monitored for severity of symptoms, spread of virus throughout entire plant, and product recovery. At 10-15 days post infection (dpi) leaf samples from infected leaf samples are harvested to assess the presence of full-size recombinant protein. A portion of the harvested material (10 leaves) is frozen in −80° C. and retained as a seed inoculum for the subsequent production scale-up of selected constructs. The rest of the tissue is processed immediately.

At 15-20 days post infection (dpi) recombinant proteins are recovered. The procedure is optimized to recover optimum quantities of high purity product (90-95% purity). Once products with the expected sizes are recovered and serological identity (recognized by specific antibodies using Western blot and ELISA) determined, the stability of the constructs is tested by three passages on healthy plants. Problems with assembly, recovery or stability of recombinant virus with proteins in the size range employed are manage at the level of nucleotide sequence or amino acid sequence by changing the conditions of infection or by using an alternative host plant.

Establishing seed-lot and procedures for medium scale production. When stage 1 is completed, a small quantity (100 ul) of in vitro synthesized transcripts of the recombinant constructs is prepared and used to inoculate 10 plants. Within 10-12 days after inoculation the leaves are harvested, tested for the presence of protein by Western blot, and stored at −70° C. as seed material. A portion of this material (3-4) is used to inoculate 150-200 plants (1-2 kg of fresh tissue). Fifteen to twenty days after inoculation, recombinant protein is recovered and used for functional studies. An average of 60 mg of product per batch is expected.

Plant inoculation and product recovery. In vitro transcripts of recombinant virus containing target are synthesized using T7 RNA polymerase and purified plasmid DNA. Transcripts are capped using the RNA cap structure analog m7G(5)ppp(5)G. For inoculation, a mixture of in vitro transcription products is applied to the leaves of the target host plants after abrading the leaf surface with carborundum and gently rubbing on the leaf surface to spread the inoculum and further abrade the surface. The purity and activity of the plant produced protein are tested. The antibody binding capacity of the plant-produced antigens is tested by ELISA during and after purification of the proteins.

Protein expression in Nicotiana benthamiana: To express full-length proteins in virus-infected plants, we used a functional complementation approach. During plant-to-plant passages, the amount of Alfalfa mosaic virus (Av)/target in the infected tissue gradually decreased and after the third transfer, only Av/A4 was detectable. (This is an advantage from an environmental safety point of view.) Using this approach, we could express an average of 100 μg of target per gram fresh tissue. An important component of this system, Alfalfa mosaic virus CP, is unique in its ability to encapsidate the genomic RNAs of unrelated viruses into infectious particles in the infected host. This unique ability of Alfalfa mosaic virus CP is exploited to engineer hybrid vectors that specifically target selected crop species.

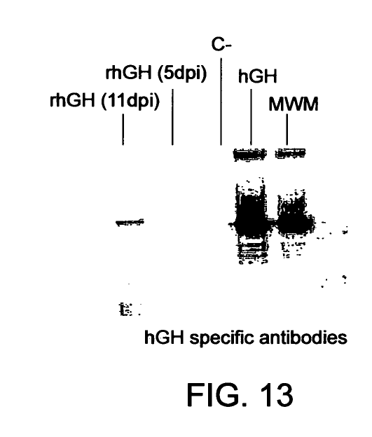

Expression of recombinant human growth hormone in Nicotiana benthamiana plants inoculated with Av/A4 and Av/A4hGH was analyzed by Western Blot (see FIG. 13). Nicotiana benthamiana plants were inoculated with in vitro transcripts and the plants monitored for production of hGH. No signal specific to the protein could be detected at 5 dpi (days post inoculation), although at 11 dpi we could detect a signal for hGH in the inoculated plants. Protein extracts from leaves infected systematically as described herein were separated by electrophoresis on a 12% SDS-polyacrylamide gel, transferred to a nitrocellulose membrane and reacted with protein-specific antibodies. FIG. 13 is a Western Blot of hGH produced in N. benthamiana plants infected with in vitro transcripts of 125C/hGH. Samples were analyzed 24 hours post inoculation. 1 μg of purified hGH was loaded as standard. MWM is molecular weight marker. The arrow in FIG. 13 points toward the hGH band on the blot detected by hGH-specific antibodies.

### Example 4

### Expression of Growth Hormone in Plants

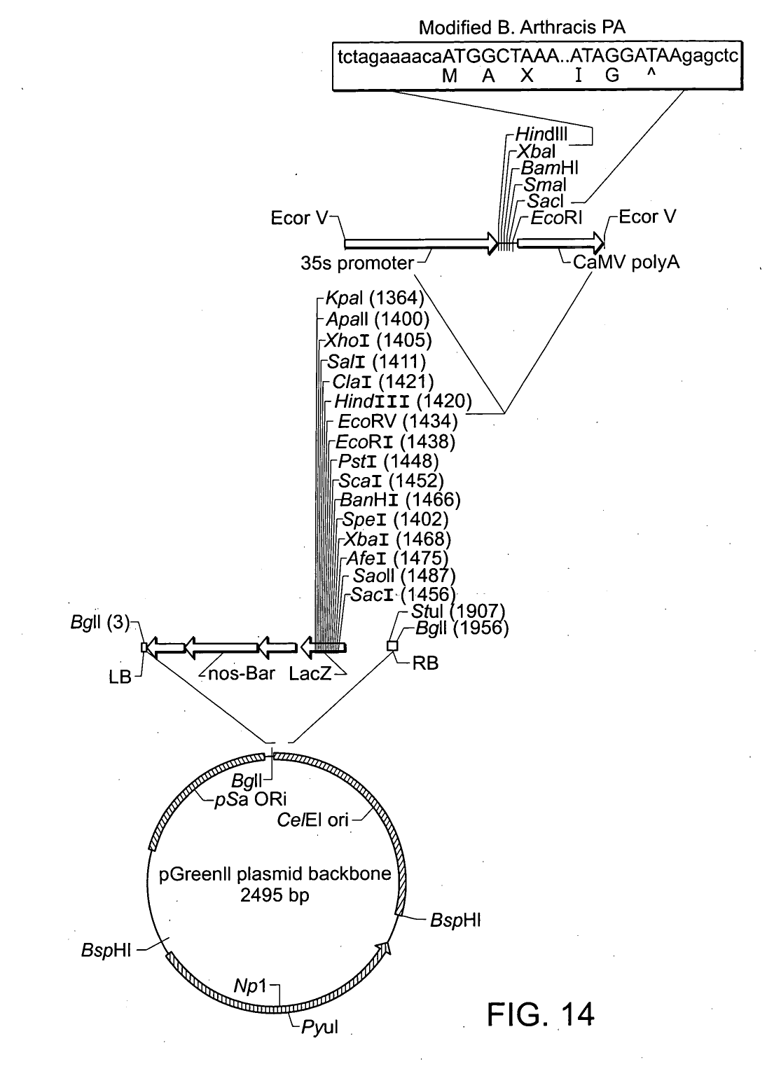

Transformation vector: The binary vector pGREENII 0229 (Hellens et al., Plant Molecular Biology (April 2000) 42(6):819-832) is used for plant transformation (see FIG. 14). This plasmid includes the following components. 1) The pGREENII plasmid backbone includes sequences necessary for replication in Escherichia coli and Agrobacterium tumefaciens. 2) The Agrobacterium tumefaciens T-DNA left and right border (LB and RB) sequences are necessary for integration of all sequences between LB and RB into the plant genome. 3) The npt gene encoding kanamycin resistance for selection of Escherichia coli and Agrobacterium tumefacients tranformants. 4) The nos-bar gene, which encodes resistance to the herbicide Bialaphos (resistance to Bialophos is used to select transgenic plants. The nos-bar gene is transcribed from the Cauliflower mosaic virus (CAMV) 35S promoter with constitutive activity in plants, and transcription of the gene is terminated using the CaMV terminator. 5) Expression of vir genes PA and LF are driven either by the CAMV 35S promoter as shown in FIG. 14, or by the HSP18.2 promoter (GenBank accession # X17295, locus At5g59720) for the Arabidopsis thaliana low molecular weight heat shock protein (Matsuhara et al., The Plant Journal: for Cell & Molecular Biolog (April 2000) 22(1):79-86). Transcriptional termination is mediated by the terminator of the nopaline synthase gene (nos). The 35S and HSP18.2 promoters where chosen for their differing activities. The 35S promoter is constitutively active in most plant tissues. In many cases the 35S promoter drives high-level expression of the protein encoding growth hormone. By contrast the HSP18.2 promoter is nearly inactive unless the plants are challenged by heat shock. High-level expression of some proteins can only be achieved using an inducible promoter system.

Vector construction and plant transformation. The vectors are constructed using DH5, a laboratory strain of Escherichia coli. The construction is analyzed by restriction endonuclease mapping and sequencing. After construction is confirmed the vector is used to transform Agrobacterium tumefaciens strain GV3101, lacking a native Ti plasmid and suitable for transfer of the TDNA of binary vectors into plants. Brassica juncea plants are transformed by introduction of GV3101 carrying the transformation vector into flowers by any available transformation method.

Selection and analysis of transgenic plants. After transformation of Brassica juncea with Agrobacterium tumefaciens strain GV3101 carrying the transformation construct the plants are allowed to progress through their normal developmental program. Within 25 days, mature seeds are produced, dried, and harvested. The seeds are sown in potting soil and seedling plants are grown for 15 days. The leaves of each plant are then sprayed with a 0.0058% (w/v) solution of Bialophos (FINALE, Farnam, Inc., Phoenix, Ariz.). Sensitive, non-transgenic plants are typically killed within 5 to 7 days. Bialophos-resistant plants are evident by their green and healthy appearance. The putative transgenic plants are confirmed and analyzed using a series of protocols. Polymerase chain reaction using oligonucleotide primers specific for the construct are used to confirm the presence of the TDNA in putative transgenic plants. The presence of the TDNA is then be examined by blotting of genomic DNA using a specific DNA probe to the target gene sequence. Finally, expression of the protein encoding growth hormone is examined by SDS-PAGE followed by staining with Coomassie Blue or immunoblotting. The protocol for analysis of protein expression differs depending upon the construct used to produce the transgenic plant. Plants carrying 35S promoter constructions are analyzed directly since expression from this promoter is constitutive. Plants carrying the HSP18.2 promoter are heat shocked before analysis. The kinetics of recombinant protein expression might differ in different transgenic plants. Ultimately, the induction conditions are optimized for each using standard methods. However, for initial analysis it is possible to carry out a standardized heat shock protocol that is established for expression of other recombinant proteins in Brassica juncea.

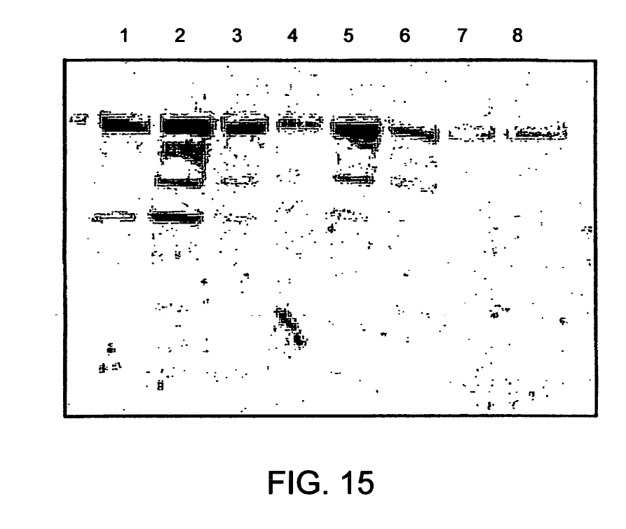

FIG. 15 shows an immunoblot of transgenic Brassica juncea expressing human growth hormone (hGH) under control of the HSP18.2 promoter. Transgenic Brassica juncea were grown in potting mix. A leaf weighing 1 to 3 grams fresh weight was detached from the plant and placed in a petri dish containing a filter paper moistened with water. The petri dish is covered and placed in a high humidity, 37° C. incubator for 1.5 hours. The petri dish containing the leaf was then removed from the incubator and placed for 5 hours in a 24° C. growth chamber under fluorescent lights at an intensity of 100 μmol photons m⁻² s⁻¹. The plant material was then harvested and analyzed by immunoblotting using a monoclonal antibody against hGH (Sigma Chemical Co., Product #G-8523). The lower band is the 16 kDa recombinant hGH. This band is not observed before heat shock. Lane 8 shows the results from a transgenic plant transformed with the vector alone. The higher molecular weight band is a non-specific reaction with the horseradish peroxidase-linked secondary antibody used to detect immune-complexes.

After an initial screen for expression a more detailed analysis of expression is carried out. An ELISA method is used to quantitate the level of expression. Further analysis is carried out on subsequent plant generation. In order to avoid the need to select for transgenic plants in subsequent generations it is ultimately necessary to isolate transgenic lines that are non-segregating for the TDNA construct. This is accomplished by self-pollinating the primary transgenic plants, raising the secondary generation plants to maturity, and testing the tertiary generation for segregation of Bialophos resistance. Secondary generation individuals are identified that are non-segregating. Thereafter, progeny of non-segregating plants are bulked and used for analysis of production scale conditions (e.g., 1,600 Kg per month of dried biomass). For production scale the growth and induction conditions are optimized for plants grown as seedlings. At this point it is also desirable to characterize the insertion site and TDNA copy number of elite lines and to characterize expression at the level of mRNA expression.

### Example 5

### Construction of Viral Vectors

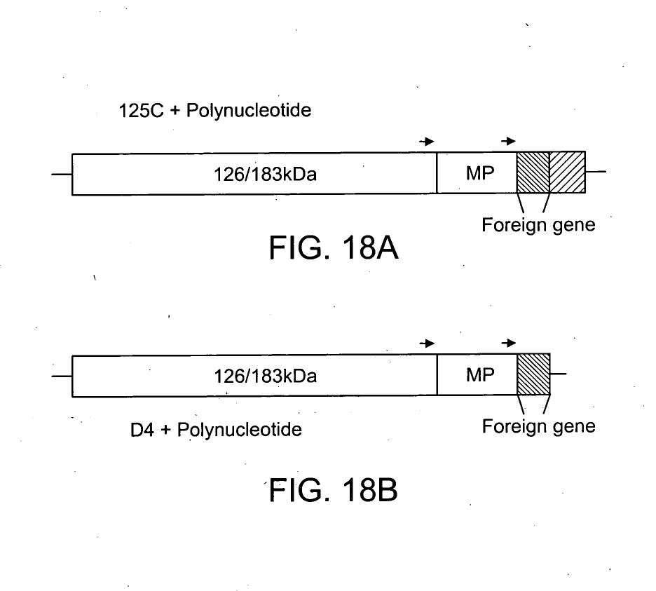

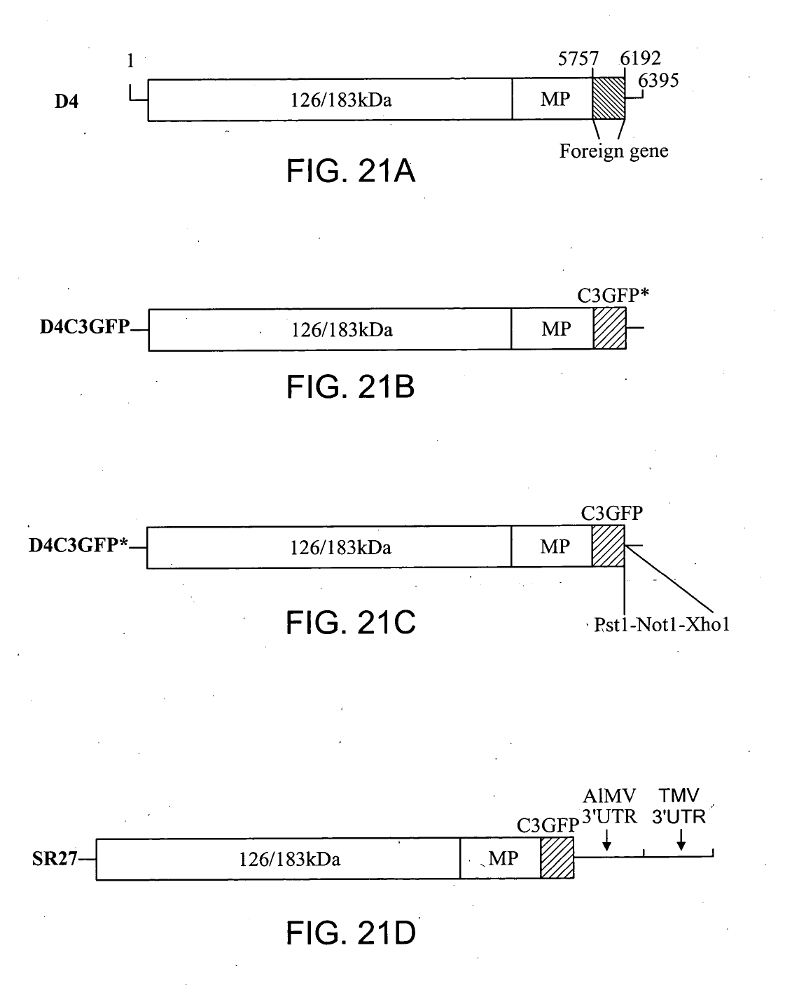

We have constructed a vector based on the Tobacco Mosaic Virus that is adapted for insertion of a polynucleotide encoding growth hormone to generate a producer vector according to the present invention. Specifically, we have generated vectors that are deficient in CP production (see FIGS. 18 and 21; vector D4 is represented with a generic polynucleotide inserted; vector SR-27 and related vectors are derived from D4 as described further in Example 7). We have demonstrated that infection with such vectors is limited to locally inoculated leaves. These vectors depends upon a second vector for systemic movement.

We have used a protoplast system to test vector replication, replication-dependent stability, and efficacy of protein production. We have also inoculated Nicotiana benthamiana plants to test the cell-to cell movement and stability of the vector, and have demonstrated systemic infection when this vector is administered together with a wild type AIMV vector including an AIMV CP gene. An AIMV-based vector referred to as Av/A4, which contains a functional AIMV coat protein gene, has been constructed. We have established a tobacco protoplast system and tested the components of this vector. Western blot analysis demonstrated accumulation of virus coat protein, indicating infection of protoplasts and verifying that we are able to reliably detect expression of CP in our protoplast system.

We have successfully infected two host plant species, Nicotiana benthamiana and pepper plants. Western blot analysis of upper leaves (not initially infected) analyzed 12 days after inoculation demonstrated AIMV CP protein is readily detectable, indicating that we are able to reliably detect expression of CP in infected plant hosts.

### Example 6

### Expression of a Polynucleotide Encoding Human Growth Hormone

FIG. 18 shows two TMV-based vectors, 125C and D4, that were engineered to accept insertion of a polynucleotide of interest, following insertion of the polynucleotide (indicated as “foreign gene”). 125C includes TMV coat protein sequences (i.e. sequences extending downstream from nucleotide 5757 of the TMV genome) that contain a cis element that may be required for optimal replication. We inserted the gene for human growth hormone (hGH) into each of these vectors between the Pac1 and Xho1 sites. An AUG was introduced in the 5′ primer used to amplify the gene from a plasmid, and the amino acids KDEL were introduced at the 3′ end of the coding sequence in order to enhance translation due to retention in the ER. HGH was cloned with and without its native leader sequence; hGH2 lacks the leader and hGH4 includes the leader.

Primer SR22 (5′-CCG TTAATTAATG TTC CCA ACT ATT CCA) was used to clone hGH without its leader, and introducing a Pac1 site at the 5′ end; primer SR23 (5′-CCG TTAATTAATG GCA ACT GGA TCA AGG) was used to clone hGH with its leader. Primer SR24 (5′-CGG CTC GAG TTA AAA ACC ACA TGA) was used to clone the hGH gene without KDEL and introducing a Xho1 site at the 3′ end; primer SR25 (5′-CGG CTC GAG TTC ATC TTT AAA ACC TGA TCC) was used to clone the gene with KDEL.

In vitro transcripts of the 125C vector constructs including hGH were prepared by linearizing approximately 20 ug of DNA in 100 uL volume. Extent of linearization was assessed by gel electrophoresis of a 2 uL sample. Linearized DNA was cleaned using a PCT purification kit, from which it was eluted in 50 uL. A transcription mix was prepared in a 25 uL volume with 2.5 uL of 10×T7 buffer, 2.5 uL of 100 mM DTT, 0.5 uL of RNAsin (Promega), 1.25 uL NTP mix (20 mM A, C, U; 2 mM G; Pharmacia-Amersham); 1.25 uL Cap (5 mM diguanosine triphosphate; Pharmacia-Amersham), and 4 uL 25 mM MgCl₂. The mixture was warmed to 37° C. for 1 minute. 1.5-2 ug DNA were added in 12 uL of water, and the combination was warmed at 37° C. for 2 minutes. 1 uL of T7 polymerase (50 U/uL; New England Biolabs) was added, and the reaction were incubated for 15 minutes. 2 ul of 12.5 mM GTP were added by touching the tip of a pipette to the liquid (do not pipette up and down). The reaction was incubated at 37° C. for 1 h 15 minutes. A 2.5 uL aliquot was visualized on a gel; the remainder was frozen.

The resulting constructs were tested in both a protoplast system and in intact plants. Tobacco protoplasts were inoculated with each the various transcripts via electroporation (i.e., plants were inoculated with transcripts from individual constructs, not with a combination of different transcripts). Plant leaves were inoculated by diluting the transcription reaction through addition of 25 uL water and 50 uL FES. Plants were dusted with carborundum powder that acts as an abrasive. 25 uL aliquots of the transcription reaction/FES solution were then gently rubbed on the surface of each of two leaves. The plants were then maintained in the growth room at 21° C. under 12 hour light and 12 hour dark conditions.

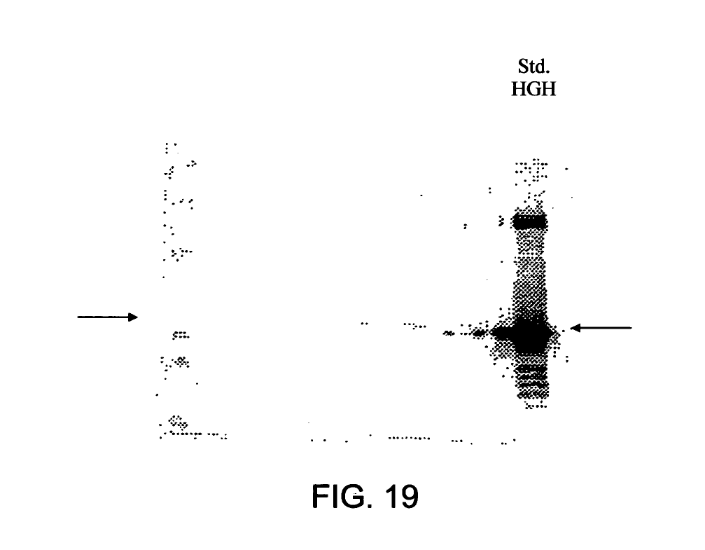

Nicotiana tabacum suspension protoplasts were harvested at two time points: 24 and 48 hours post inoculation, so that each aliquot contained 500,000 protoplasts. Approximately 2 million protoplasts were used per inoculation of 25 uL transcript. The protoplasts were pelleted by centrifugation and the pellet was resuspended in 50 uL buffer (a mixture of Bradley's protein extraction buffer and Laemmli loading buffer). The samples (10 uL) were analyzed by PAGE followed by Western blot hybridization analysis using antiserum to hGH from chicken and anti-chicken IgG conjugated to alkaline phosphatase. Standard hGH was run as a standard. NBT-BCIP was used to develop the blots. FIG. 19 shows the results of the experiment. The results indicate that a higher yield of hGH was obtained from tobacco suspension protoplasts at 24 h than at 48 h post inoculation. The position of the band corresponding to hGH from infected protoplasts indicates a slightly higher molecular weight than standard hGH. This could be due to the KDEL sequence attached to the 3′ end of the hGH protein.

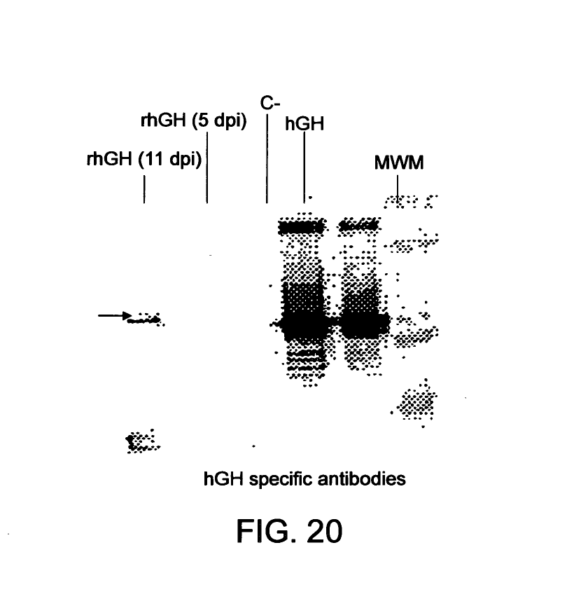

Nicotiana benthamiana plants were also inoculated with in vitro transcripts, and the plants were monitored for production of hGH. No signal specific to the protein could be detected at 5 dpi, although at 11 dpi we could detect a signal for hGH in the upper leaves of inoculated plants (FIG. 20).

### Example 7

### Co-infection and Cross-Complementation of Viral Vectors

This example demonstrates that a coat protein defective TMV-based expression vector can be complemented by an AIMV vector that supplies CP in trans.

D4C3GFP is a TMV-based expression vector that is deficient in CP production (Shivprasad et al., 1999: TTT-GFP) as a result of deletion of the TMV CP coding region and the its replacement with the C3GFP gene, which is placed under the control of the TMVCP subgenomic promoter (see FIG. 21b). The C3GFP gene was recloned into D4 by overlapping PCR to eliminate the Nco1 and Xho1 sites in the C3GFP nucleotide sequence to facilitate further cloning steps. A polylinker PstI-NotI-XhoI was introduced at the 3′end of C3GFP gene. The PCR product digested with PacI-XhoI was cloned into D4 resulting in the version of D4C3GFP shown in FIG. 21 c.

The primers we used to modify the C3GFP gene and eliminate Nco1 and Xho1 sites are:

1) C3GFP.Pac1.For(N):
GGGAG.ATCTT.AATTA.ATGGC.TAGCA.AAGGA.GAAGA.A
(36 nt)
2) C3GFP.Xho1.Rev(N):
CCCCT.CGAGC.GGCCG.CTGCA.GTTAT.TTGTA.GAGCT.CATCC.
ATGCC (45 nt)
3) C3GFP.Nco1.For:
GTTCC.CTGGC.CAACA.CTTGT.CAC (23 nt)
4) C3GFP.Nco1.Rev:
TAGTG.ACAAG.TGTTG.GCCAG.GG (22 nt)
5) C3GFP.Xho1.For:
GGACA.CAAAC.TGGAG.TACAA.CTATA (25 nt)
6) C3GFP.Xho1.Rev:
AGTTA.TAGTT.GTACT.CCAGT.TTGTG (25 nt)
7) (BglII)-PacI
> AUG...HindIII...NcoI...Ndel...BsrGI...MluI...
XhoI...BamHI...MfeI(MunI)...SalI...SacI...TAA <
PstI...NotI...XhoI

Three constructs that contained full-length or portions of the 3′-untranslated region (3′ UTR) of AIMV RNA3 were then generated. In each of these constructs, sequences encoding C3GFP under control of the subgenomic TMV CP promoter were present upstream of AIMV RNA3 3′-UTR sequences (either full-length or a portion of the UTR), to allow us to precisely identify the sequences of the AIMV RNA3 3′ UTR required for assembly and movement of TMV genomic RNA (either in trans or in cis). The RNA3 sequences were inserted between the Not1 and XhoI sites of the new D4C3GFP vector as Not1-Sal1 fragments, resulting in the constructs SR25 (nts 1859-1941 of RNA3), SR26 (nts. 1859-1969) and SR27 (nts. 1859-2037) (FIG. 21d). In addition to sequences from the AIMV RNA3 3′ UTR, SR25, SR26, and SR27 also include sequences from the TMV 3′ UTR (i.e., the UTR from the TMV genomic transcript) downstream of the inserted AIMV sequences. These sequences are TMV nucleotides 6192-6395, as in the D4 construct. The TMV-based viruses (SR25, SR26, and SR27) are defective in long-distance movement because the TMV coat protein is essential for effective phloem-mediated long distance transport and systemic infection of TMV.

The primers used to generate D4-based constructs with AIMV RNA3 3′-UTR sequences were:

1) SR-52 5′ primer with Xho1-Pst1 sites at nt 1859
(plus sense)
5′-CCGCTCGAGCTGCAGTGTACCCCATTAATTTGG-3′
2) SR-53 3′ primer at nt 1941 of AIMV RNA3 with
Not1-Sal1 sites: minus sense
5′-CGGGTCGACGCGGCCGCGAATAGGACTTCATACCT-3′
3) SR-54 3′ primer with Not1-Sal1 sites at nt 1969
of AIMV RNA3: minus sense
5′- CGGGTCGACGCGGCCGCAATATGAAGTCGATCCTA-3′
4) SR-55 3′ primer with Not1-Sal1 sites at nt 2037
(minus sense)
5′-CGGGTCGACGCGGCCGCGCATCCCTTAGGGGCATT-3′.

The resulting plasmids were then transcribed using T7 polymerase and the in vitro transcripts used to inoculate Nicotiana benthamiana plants. In vitro transcripts of SR25, SR26, SR27, and a wild type AIMV construct were prepared by linearizing approximately 20 ug of DNA in 100 uL volume. Extent of linearization was assessed by gel electrophoresis of a 2 uL sample. Linearized DNA was cleaned using a PCT purification kit, from which it was eluted in 50 uL. A transcription mix was prepared in a 25 uL volume with 2.5 uL of 10×T7 buffer, 2.5 uL of 100 mM DTT, 0.5 uL of RNAsin (Promega), 1.25 uL NTP mix (20 mM A, C, U; 2 mM G; Pharmacia-Amersham); 1.25 uL Cap (5 mM diguanosine triphosphate; Pharmacia-Amersham), and 4 uL 25 mM MgCl₂. The mixture was warmed to 37° C. for 1 minute. 1.5-2 ug DNA were added in 12 uL of water, and the combination was warmed at 37° C. for 2 minutes. 1 uL of T7 polymerase (50 U/uL; New England Biolabs) was added, and the reaction was incubated for 15 minutes (SR25, SR26, SR27 constructs) or 2 hours (AIMV construct). 2 ul of 12.5 mM GTP were added by touching the tip of a pipette to the liquid (do not pipette up and down). The reaction was incubated at 37° C. for 1 h 15 minutes (SR25, SR26, SR27 constructs) or 30 minutes (AIMV construct). A 2.5 uL aliquot was visualized on a gel; the remainder was frozen.

Plant leaves were inoculated with SR25, SR26, or SR27 by diluting the transcription reaction through addition of 25 uL water and 50 uL FES. Plants were dusted with carborundum powder that acts as an abrasive. 25 uL aliquots of the transcription reaction/FES solution were then gently rubbed on the surface of each of two leaves. The plants were then maintained in the growth room at 21° C. under 12 hour light and 12 hour dark conditions.

Two weeks post inoculation, when SR25, SR26, SR27 had spread in the inoculated leaves, which was visualized by exposing the plants to long-wave ultraviolet light (366 nm), the same leaves were inoculated with wild type AIMV transcripts as described for the TMV-based vectors.

Two weeks post infection with AIMV, diffuse GFP fluorescence could be observed in upper leaves of plants infected with SR27 and AIMV but not with SR25 or SR26 and AIMV, which demonstrates spread of virus into the upper un-inoculated leaves. Lack of fluorescence in the upper leaves indicates that virus infection was limited to locally inoculated leaves. These results indicate that the CP-deficient TMV-based virus (SR27) containing the GFP transgene moved through the phloem into the upper leaves with the help of AIMV. Generally (e.g., in the absence of trans-complementation from another virus) D4C3GFP only moves into the major veins of the upper leaves 40-45 d.p.i., and SR27 requires similar or even longer periods of time to move into the upper leaves in this system. This result indicates that AIMV can be used as a source for the coat protein that will complement and allow movement of a viral vector that is deficient in one or more coat protein components systemically and provide expression of foreign proteins, including complex proteins such as antibodies. The complementing CP components can be from related (other alfamoviruses, ilarviruses, bromoviruses) or unrelated viruses (TMV, CMV, etc.).

Similar methods described above have been use to generate constructs related to SR27 but containing the hGH gene.

### Example 8

### Construction of Recombinant Plant Virus Vectors

We employed vectors based on the Tobacco Mosaic Virus that are adapted for insertion of a polynucleotide encoding growth hormone to create a vector for use in generating clonal root lines, clonal root cell lines, clonal plant cell lines, and/or clonal plants that express a polynucleotide encoding growth hormone according to the present invention. FIG. 1 shows a schematic diagram of a TMV-based vector, D4, that was engineered to accept insertion of a polynucleotide of interest (Shivprasad et al., Virology, 255(2):312-23, 1999), and illustrates insertion of various polynucleotides of interest into the vector. D4 contains a deletion of the TMV coat protein (CP) coding sequences but retains the TMV CP subgenomic promoter and the TMV 3′ untranslated region (UTR), as indicated on the figure. The 126 and 183 kD proteins are required for TMV replication. The 30 kD protein is movement protein (MP), used for cell-to-cell movement. D4 contains Pac I and Xho I sites downstream of the CP subgenomic promoter, providing a site for convenient insertion of a polynucleotide encoding growth hormone.

D4C3GFP was prepared as described in Example 7 above. Viral vectors in which polynucleotides of interest (e.g., GFP, hGH,) are inserted into SR25, SR26, and/or SR27 are in the process of being tested for generation of clonal root lines, clonal plant cell lines, and clonal plants as described herein.

To generate TMV-based constructs suitable for expression of human growth hormone (hGH) we inserted the gene for hGH into the D4 vector between the Pac1 and Xho1 sites. An AUG was introduced in the 5′ primer used to amplify the gene from a plasmid, and the amino acids KDEL were introduced at the 3′ end of the coding sequence in order to enhance translation due to retention in the ER. For the experiments described herein, hGH was cloned without its native leader sequence, resulting in D4-hGH, which was used in the experiments described herein.

Primer SR22 (5′-CCG TTAATTAATG TTC CCA ACT ATT CCA) was used to clone hGH without its leader, and introducing a Pac1 site at the 5′ end; primer SR23 (5′-CCG TTAATTAATG GCA ACT GGA TCA AGG) was used to clone hGH with its leader. Primer SR24 (5′-CGG CTC GAG TTA AAA ACC ACA TGA) was used to clone the hGH gene without KDEL and introducing a Xho1 site at the 3′ end; primer SR25 (5′-CGG CTC GAG TTC ATC TTT AAA ACC TGA TCC) was used to clone the gene with KDEL.

### Example 9

### Generation and Testing of Clonal Root Lines Expressing GFP

Synthesis of viral transcripts and viral infection. In vitro transcripts of vector D4C3GFP, described above, which contains an open reading frame encoding GFP under control of the TMV CP subgenomic promoter, were synthesized using T7 polymerase. Approximately 10 μg of DNA was linearized with 30 units of KpnI overnight in a reaction volume of 100 μl. Four μl of the restriction digest was used to produce in vitro transcripts using the AmpliCap T7 High Yield message Maker Kit (Epicentre) according the manufacturers recommendations. Transcripts from one such reaction were used to infect six-week-old Nicotiana benthamiana plants by manually applying the transcripts dissolved in FES onto young, fully expanded leaves.

Agrobacterium rhizogenes stimulated root generation. Agrobacterium rhizogenes strain A4RSII was grown to OD₆₀₀ 0.8-1. Bacterial cells were pelleted and resuspended in MS-2 medium (MS salts, 2% sucrose, 10 mM MES, pH 5.5) to a final OD₆₀₀ of 0.5. Acetosyringone was added to a final concentration 200 μM 1 hour before transformation. Local or systemically infected leaves of Nicotiana benthamiana were harvested 5-14 days after inoculation with transcript. Leaves were surface sterilized for 6 min with 10% Clorox and washed several times with sterile distilled water.

Surface sterilized leaves of N. benthamiana were cut into pieces ˜1 cm². They were dipped into bacterial suspension for 5 min, drained on filter paper and placed on the surface of solidified MS-2 medium. Plates were kept under dim light conditions at 24° C. for 48 hours. After 48 hours the excess Agrobacterial suspension was removed, and leaf explants were placed on solid hormone free K₃ (Kao K. N. and Michayluk M. R., Plants, 115:355-367, 1974.) modified according to Nagy and Maliga, (Nagy J. J. and Maliga P., Z.Pflanzenphysiol. 78:453-455, 1976) and Menczel et al. (Menczel L., Nagy F., Kiss L. R. and Maliga P., Theor. Appl. Genet. 59:191-195, 1981) medium. Plates were maintained at 25° C. with a 16 hr day/8 hr night light regime.

Three weeks after transformation, hairy roots were cut off and placed in a line on solid hormone free K₃ medium. Four to six days later, the most actively grown roots were isolated and transferred to liquid K₃ medium in individual Petri dishes. The roots were cultured on a rotary shaker at 24° C. and subcultured ˜weekly by dissecting and harvesting a portion of the root mass and transferring the harvested roots to a Petri dish containing fresh K₃ medium. Roots were screened for the presence of the protein encoding growth hormone by Western blot analysis and/or by fluorescence under UV light, depending on the particular polynucleotide of interest.

Western blot assays. For Western blot assays 10 mg of fresh root material was placed into an Eppendorf tube and homogenized in 50 ul of phosphate buffer, followed by the addition of 20 ul of 5× loading buffer and 10 minutes of boiling. After boiling, the homogenate was centrifuged for 5 to 10 minutes to clear the debris. Following centrifugation, 10 ul of sample was loaded on an SDS polyacrylamide gel, and proteins were separated by electrophoresis. Commercially available GFP protein (5 ng) (BD Biosciences Clontech) was loaded as a positive control. Leaf samples (10 mg) from N. benthamiana plants systemically infected with the same vector (D4C3GFP) were harvested at the time of peak expression, and an extract was prepared in an identical manner as described above for the root material and loaded on the gel for comparison with the root cell lines. Upon completion of electrophoresis proteins were electroblotted onto a nylon membrane, blocked using casein and reacted with GFP-specific antibodies (BD Biosciences Clontech). Proteins reacting with antibodies were visualized using a chemiluminescent substrate.

Western blot analyses demonstrated GFP production in 3 clonal root lines derived from plant cells into which a viral vector whose genome contains a gene that encodes GFP under control of the TMV CP promoter (D4C3GFP) was introduced. GFP expression in the clonal root lines after 30 and 60 days of propagation in culture (i.e., 30 and 60 days after separation of the root from the leaf from which it was derived). These results demonstrate that the clonal root lines maintain high level expression of a protein of interest (GFP) over an extended period of time, indicating the stability of the viral transcript in the clonal root lines.

It is noted that Western analysis demonstrated expression of GFP throughout all portions of the root mass. However, when screened using a visual approach, expression generally appears stronger in the more mature portions of the root mass than in the growing tips, where cell division is proceeding rapidly. This appears to be due both to the time required for new cell to synthesize sufficient GFP for visibility and to the fact that when viewed from above, one is looking through multiple layers of cells in the thicker portion of the roots. It is also noted that the most mature portions of the roots may become somewhat “woody”, which can obscure visual detection of GFP.

### Example 10

### Generation and Testing of Clonal Root Lines Expressing hGH

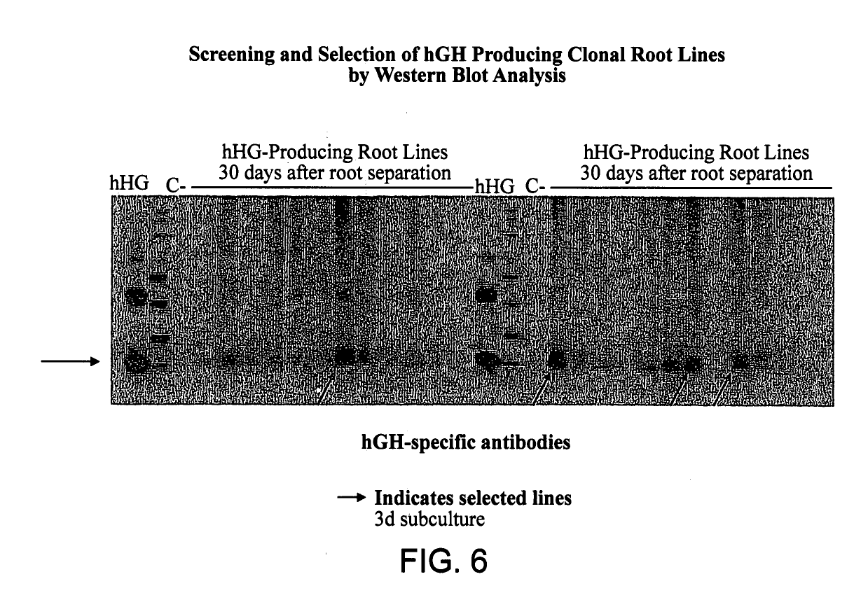

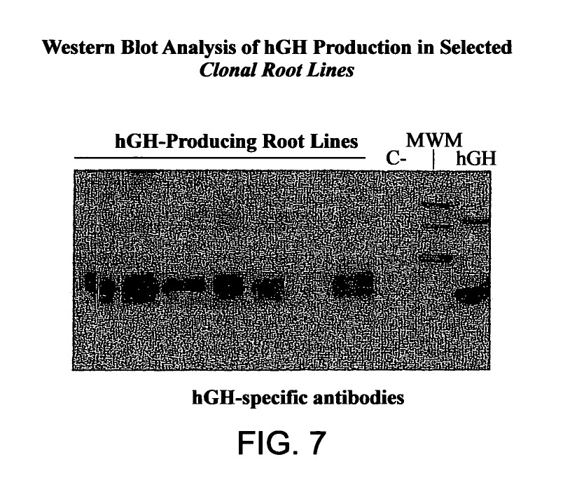

N. benthamiana plants were inoculated with a TMV-based vector, D4-hGH, containing an open reading frame encoding hGH under control of the TMV CP subgenomic promoter. Hairy roots were obtained and subcultured essentially as described in Example 9. Two weeks after separation from leaf discs, during the third round of subculture, the segments of roots were analyzed for hGH expression by Western blot assay essentially as described in Example 2. See FIG. 6. Five ng hGH protein (Research Diagnostics) was used as a control in all Western blots in which expression of hGH was tested. Anti-hGH antibodies were from Research Diagnostics. As can be seen from FIG. 6, up to 80% of the clonal root lines had detectable levels of hGH. We selected the highest producers and propagated them further. After 10 passages (subculturings), samples were taken and analyzed for hGH accumulation. FIG. 7 shows a Western blot, demonstrating that the clonal root lines maintained stable expression of hGH after 10 passages in which hGH expression in selected lines was several fold higher (250 ug/gram fresh root tissue) than that in leaves infected with the same virus construct (70 ug/gram fresh leaf tissue) when compared by Western blot.

### Example 11

### Generation and Testing of Clonal Plant Cell Lines

Clonal plant cell lines were derived by introducing a TMV-based viral vector containing an open reading frame that encodes target protein under control of the TMV CP subgenomic promoter into BY-2 cells. Cell culture and electroporation. Cell lines derived from Nicotiana tabacum cv Bright yellow (BY-2) were maintained in MS medium (Murashige T. and Skoog F., Physiol. Plant. 15:473-497, 1962) supplemented with 0.2 mg/l 2,4-D and 0.1 mg/l Kinetin, 20 mM MES, pH 5.6-5.8 on a shaker, 140 rpm at 25° C., and subcultured weekly. For electroporation, protoplasts were generated from cells that had been subcultured for 3-4 days. Cells were spun at 1000 rpm for 8 min, washed 2× with Mannitol 0.4M and MES 20 mM, pH 5.5. Cells were then taken to 30-50 ml with filter sterilized protoplasting solution: 0.4M mannitol, MES 20 mM, pH5.5, Cellulase Onozuka RS (Yakult Honsha Co.) 1%, Pectolyase Y23 (Seishin Pharmaceutical Co.) 0.1%. Cells were incubated in 250 ml flasks at 25° C. for 20-25 min. The protoplast solution was filtered through a 100/μm sieve, spun at 700 rpm for 6 min, and washed 2× with ice-cold 0.4M Mannitol. Protoplasts were counted using a hemacytometer and resuspended in electroporation buffer: 10 mM HEPES, 150 mM NaCl. 5 mM CaCl₂, 0.4M mannitol, pH 7.2 to a final concentration 1×10⁶ protoplasts/ml.

Transcript (25-30 μl) was placed into an electroporation cuvette, 0.4 cm (Biorad) kept on ice, and after 10-15 min was mixed with 0.5 ml of protoplast suspension by Pasteur pipette and immediately used to electroporate cells. Electroporation was performed using a Biorad Gene Pulser at 250 volts and 175 capacitance. Electroporated protoplasts were resuspended in 8 ml of PBS buffer containing 0.4 M mannitol and maintained for formation of the cell wall.

Enrichment for stable producer cell lines. Within 4-5 days following electroporation, dividing cells were diluted and sampled (10 ul of infected cells into 100 ul of medium) to enrich for cells that expressed the polynucleotide of interest (target molecule) at high levels. The diluted cells were spotted onto individual sections of a Petri dish. Two to three weeks later each sample was tested by visual or other means (e.g., Western blot) for the presence of target molecule (e.g., GFP, hGH, etc.). Stably infected cells producing target molecule were selected for further enrichment until producer cell line is obtained.

### Example 12

### Generation and Testing of Clonal Cell Lines Expressing GFP

Clonal plant cell lines were derived by introducing a TMV-based viral vector containing an open reading frame that encodes GFP under control of the TMV CP subgenomic promoter (D4C3GFP) into BY-2 cells essentially as described in Example 11. Enrichment for cells that express GFP was performed using a visual screen for fluorescence until populations of cells (either single clonal cell lines or populations containing several clonal cell lines) that stably express GFP were obtained. Clonal plant cell lines expressing GFP are readily seen. It is noted that the droplets may contain either a single clonal plant cell line or multiple clonal plant cell lines. Single clonal plant cell lines (i.e, populations derived from a single ancestral cell) can be generated by further limiting dilution using standard methods for single cell cloning.

### Example 13

### Generation and Testing of a Clonal Plant

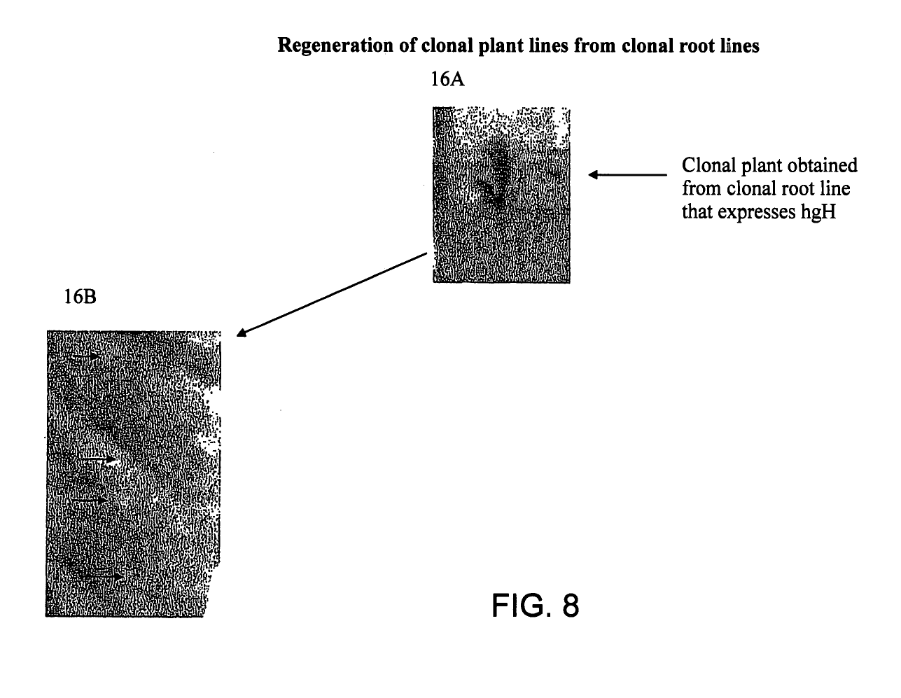

Clonal root lines expressing hGH were obtained as described in Example 10. Root cells were isolated by enzymatic digestion and cultured as described in Peres et al., Plant Cell, Tissue, and Organ Culture 65, 37-44, 2001, to generate clonal plants. FIG. 8A shows a plant that was obtained from a clonal root line. To determine whether the plant contained the viral vector, a small leaf sample was used to inoculate a tobacco variety that is a sensitive host for formation of local lesions upon viral infection. Formation of lesions within 2 days of inoculation, as indicated by arrows in FIG. 8B, indicated that the clonal plant regenerated from the clonal root line maintains active viral replication, strongly suggesting that the clonal plant also expresses hGH. Additional experiments showed that this was indeed the case.

### Example 14

### Therapeutic Activity of Plant Derived Growth Hormone

The gene for hGH was engineered into plant virus expression vectors, producer construct was selected, and N. benthamiana plant producing hGH as described in Example 3. Similarly, Western blot hybridization confirmed serological identity of plant, and levels of hGH production determined. Levels of accumulation in N. benthamiana plants was determined to yield approximately 60 μg/g of fresh leaf tissue in systemically infected leaves. The following optimized nucleic acid sequence utilized which encodes for human growth hormone (shown below):

TTAATTAAATGTTCCCAACTATTCCACTTTCTAGGCCATTCGATAACGCT
ATGCTTAGGGCTCATAGGCTTCATCAGCTTGCTTTCGATACTTACCAAGA
GTTCGAGGAGGCTTACATTCCAAAGGAACAGAAGTACTCTTTCCTTCAGA
ACCCACAGACTTCACTTTGCTTCTCTGAGTCTATTCCAACTCCATCTAAC
AGGGAGGAGACTCAGCAGAAGTCTAACCTTGAGCTTCTTAGGATTTCTCT
TCTTCTTATTCAGTCTTGGCTTGAGCCAGTTCAGTTCCTTAGATCTGTGT
TCGCTAACTCTCTTGTGTACGGAGCTTCTGATTCTAACGTGTACGATCTT
CTTAAGGATCTTGAGGAGGGAATTCAGACTCTTATGGGAAGGCTTGAGGA
TGGATCTCCAAGGACTGGACAGATTTTCAAGCAGACTTACTCTAAGTTCG
ATACAAACTCTCACAACGATGATGCTTTGCTTAAGAACTACGCACTTCTT
TACTGCTTTAGGAAGGATATGGATAAGGTGGAGACTTTCCTTAGGATTGT
GCAATGCAGATCTGTTGAGGGATCTTGCGGATTCTGACTCGAG
M F P T I P L S R P F D N A M L R A H R L H Q L A
F D T Y Q E F E E A Y I P K E Q K Y S F L Q N P Q
T S L C F S E S I P T P S N R E E T Q Q K S N L E
L L R I S L L L I Q S W L E P V Q F L R S V F A N
S L V Y G A S D S N V Y D L L K D L E E G I Q T L
M G R L E D G S P R T G Q I F K Q T Y S K F D T N
S H N D D A L L K N Y G L L Y C F R K D M D K V E
T F L R I V Q C R S V E G S C G F

Plant tissue was homogenized in PBS and produced growth hormone isolated from supernatant using different size exclusion columns selecting for molecules between 10 kD and 100 kD, followed by concentration of isolated samples. From 1.4 Kg wet weight of plant biomass we recovered a total of 8.0 mg (app. 10% of total hGH) partially purified hGH.

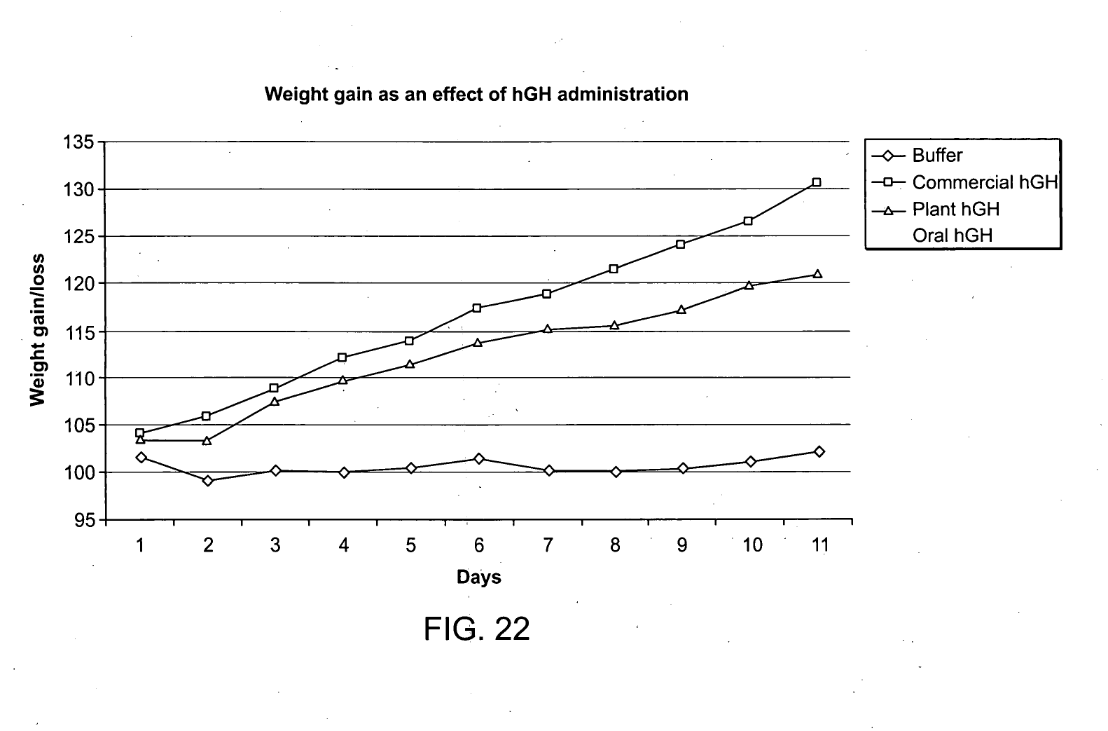

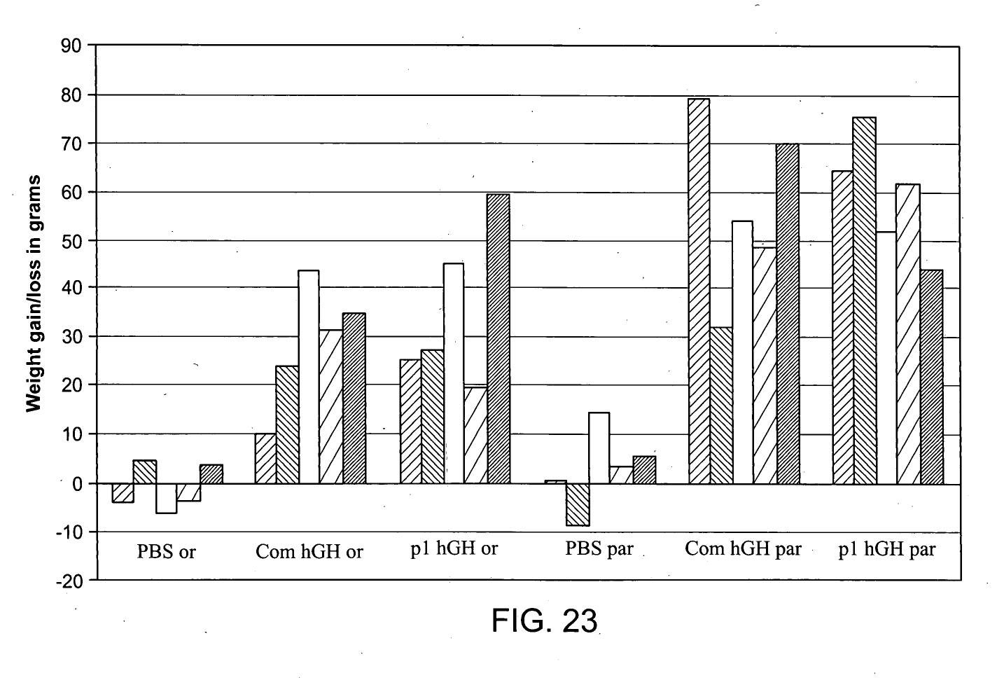

Biological activity of plant produced hGH was tested in vivo using hypoxed (hypophysectomized, i.e., having had the pituitary gland removed) Sprague Dawley rats. Lab Animals and randomly separated into 4 groups of 10 animals. Each animal was given 10 doses (one dose/day) of material:Control groups (Buffer and Commercial hGH), respectively, received 200 μl of phosphate buffer/dose/day or 60 μg of hGH in 200 μl of phosphate buffer/dose/day subcutaneously; Experimental group (Plant hGH) was given 60 μg/dose/day of Semi-Pure plant-produced hGH (in 200 μl of phosphate buffer) subcutaneously; and a group of animals received approximately 60 μg of hGH orally (I gram of systemically infected leaf tissue was homogenized and administered to each rat by gastric intubation in one dose). Following 10 hGH doses, only the groups that received commercial hGH or Semi-Pure plant-produced hGH gained weight (average 0.38, 26.31, or 17.31, buffer/commercial/plant respectively, See FIG. 22) The group of rats that received buffer or hGH orally, however, did not gain weight (average weight loss 1.38 gram). Activity of plant-produced hGH was determined to yield approximately 0.2 IU/g of fresh plant tissue or 3 IU/mg of hGH activity produced by the plant. These results are comparable to the activity of commercially manufactured hGH, indicating the plant-produced hGH has biological activity comparable to that of commercially available hGH.

### Example 15

### Production and Formulation of Growth Hormone for Oral Delivery

To determine if the plant-produced hGH would be efficient if delivered orally, we produced larger quantities of plant biomass to produce ˜400 mg of hGH, formulate for oral delivery and conduct in vivo studies in a hypoxed rat model. 250 μg hGH per rat per dose was administered by oral delivery (versus 60 μg given IP)

58 kilograms of N. benthamiana tissue infected with the TMV viral vector expressing hGH was processed. The processing involved grinding the material, clarification, ultrafiltration and chromatography to remove the brown coloration of the material. The final product was in a total of 630 mL extract. Material was aliquoted into 100 mL aliquots and kept frozen at −80° C. Material was then lyophilized, yielding a total amount of lyophilized material of 36 g, and total amount of 360 mg hGH. Emerson Pharma Services, Inc (Pennsylvania) formulated tablets that contained ˜42 μg hGH. Plant-produced material and commercially available hGH (Humatrope® somatropin, Eli Lilly) were formulated to make 600 tablets of each, each tablet containing ˜42 μg of hGH.

The tablets were prepared as follows: Blending/Filling/Coating: The lyophilized protein was incorporated at 250 micrograms/capsule. To more accurately dispense and fill capsules with this material, a blend of microcrystalline cellulose (MCC) and the protein were uniformly mixed prior to encapsulation. 30 capsules were filled with this blend. Weight variation of filled capsules is monitored and reported, and should not exceed 10%. Tablet formulation comprised: Emcompress, dicalcium phosphate (47.64%, W/W), Prosolve HD90, silicified microcrystalline cellulose (38.26%, W/W), Mg Stearate(0.99%, W/W), sodium starch glycolate (2.86%, W/W), lyophilized HGH (10.25%, W/W). The capsules were coated with an enteric polymer system for release in the small intestine. Capsules were coated with a solvent based, acrylic acid/acrylate blend for release above pH 6.0 according to the formulation in Table 1. The tablets were sent to QualTech Laboratories, Inc in New Jersey for the rat study.

TABLE 1
Coating formulation

| | | | amount | |
|---|---|---|---|---|
| Material | Manufacturer | Lot number | (grams) | % solids |
| Eastacryl 30D | Eastman | TS203050000 | 537.50 | 30% |
| Plasacryl AC | Emerson | PAC040702 | 130.00 | 20% |
| Triacetin | Tessenderlo | 6726054 | 12.00 | 100.00% |
| H2O DI | Emerson | | 320.50 | |
| Total | | | 1000.00 | |

The Eastacryl 30D is methacrylic acid and ethyl acrylate
The Plasacryl AC is triaceting and acetylated monoglyceride

Since we planned to administer 250 ug hGH per rat per dose, 6 tablets were given per dose per rat (for a total of ˜250 ug per dose per rat). Animals were treated according to the Group schedule, and weight of animals monitored. The details of the study groups are as follows:

GROUP 1: 10×Hypoxed: 6 tablets/dose/day (1 hr intervals), oral, daily for 10 days (lyophilized plant material)

GROUP 2: 10×Hypoxed: 6 tablets/dose/day (1 hr intervals), oral, daily for 10 days (Humatrope® somatropin)

GROUP 3: 10×Hypoxed: 100 ul/dose/day (60 ug), SC, daily for 10 days; (lyophilized plant material).

GROUP 4: 10×Hypoxed: 100 ul/dose/day (60 ug), SC, daily for 10 days; (Humatrope® somatropin).

Control groups:

GROUP 7: 5×Hypoxed: 100 ul of 1×PBS/dose/day, SC, daily for 10 days,

GROUP 8: 5×Hypoxed: 100 ul of 1×PBS/dose/day, by oral gavage, daily for 10 days

Hypoxed rats fed orally with plant-produced hGH or Humatrope® somatropin, manufactured by Eli Lilly, demonstrated equivalent weight gain over the period of study, demonstrating that oral delivery of hGH is effective.

This application refers to various patents, patent applications, and publications. The contents of all of these are incorporated herein by reference. In addition, the following publications are incorporated herein by reference: Current Protocols in Molecular Biology, Current Protocols in Immunology, Current Protocols in Protein Science, and Current Protocols in Cell Biology, all John Wiley & Sons, N.Y., edition as of July 2002; Sambrook, Russell, and Sambrook, Molecular Cloning. A Laboratory Manual, 3^{rd} ed., Cold Spring Harbor Laboratory Press, Cold Spring Harbor, 2001; Slater, A., Scott, N. W., and Fowler, M. R., Plant Biotechnology, Oxford University Press, 2003. In the event of a conflict between the instant specification and an incorporated reference the specification shall control.

Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the following claims.

1. A method of delivering a physiologically significant amount of growth hormone or a pharmaceutically active portion thereof to a mammalian subject comprising steps of: administering a composition comprising growth hormone or a pharmaceutically active portion thereof to the subject;
wherein the growth hormone of the composition is produced in plants and wherein a physiological effect of the administered growth hormone is elicited.

2. The method of claim 1, wherein the growth hormone is human growth hormone.

3. The method of claim 1, wherein the subject suffers from a growth hormone associated condition selected from the group consisting of hypopituitarism, Turner's syndrome, growth hormone deficiency, idiopathic short stature, short bowel syndrome, Prader Willi syndrome, and weight loss or wasting associated with HIV/AIDS.

4. The method of claim 1, wherein the composition comprises plant material.

5. The method of claim 1, wherein the composition comprising growth hormone is delivered orally or intraperitoneally.

6. The method of claim 1, wherein the growth hormone is formulated as any one of a tablet, a caplet, a gelcap, a capsule, a pill or a liquid vehicle.

7. The method of claim 6, wherein the growth hormone is formulated as a tablet.

8. The method of claim 6, wherein the growth hormone is formulated as an enterically coated tablet, caplet, gelcap, capsule, or pill.

9. The method of claim 1, wherein the physiological effect is an increase in weight.

10. A composition comprising pharmaceutically active growth hormone or a portion thereof, wherein the growth hormone is produced in a plant or a portion thereof.

11. The composition of claim 10, wherein the growth hormone is orally bioavailable in amounts sufficient to achieve a physiologically significant effect.

12. The composition of claim 10, wherein the growth hormone is formulated in any one of a tablet, a caplet, a gelcap, a capsule, a pill, or a liquid vehicle.

13. The composition of claim 11, wherein the growth hormone is formulated as a tablet.

14. The composition of claim 12, wherein the growth hormone is formulated as an enterically coated tablet, caplet, gelcap, capsule, or pill.

15. The composition of claim 10, wherein the growth hormone is human growth hormone.

16. The composition of claim 10, wherein the composition comprises plant material.

17. A method of treating a subject with growth hormone, comprising steps of: administering the composition claim 10 to the subject.

18. The method of claim 17, wherein the subject suffers from a condition selected from the group consisting of: growth hormone deficiency, idiopathic short stature, short stature associated with Turner's syndrome, growth retardation due to chronic renal disease, and neuroendocrine aging, and wherein the composition is delivered in an amount sufficient to at least in part treat the condition.

19. The method of claim 17 wherein the method of delivery is oral administration.

20.-102. (canceled)

103. A plant or portion thereof comprising a nucleic acid encoding growth hormone or a pharmaceutically active portion thereof, wherein the plant is capable of producing the growth hormone protein or pharmaceutically active portion thereof, and wherein the produced growth hormone has pharmaceutical activity when administered to a subject.


SEQUENCE LISTING (21 sequences)

SEQ ID NO: 1 - DNA, 28 bp, Artificial Sequence
  Primer
ccgttaattaatgttcccaactattcca

SEQ ID NO: 2 - DNA, 28 bp, Artificial Sequence
  Primer
ccgttaattaatggcaactggatcaagg

SEQ ID NO: 3 - DNA, 24 bp, Artificial Sequence
  Primer
cggctcgagttaaaaaccacatga

SEQ ID NO: 4 - DNA, 30 bp, Artificial Sequence
  Primer
cggctcgagttcatctttaaaacctgatcc

SEQ ID NO: 5 - DNA, 36 bp, Artificial Sequence
  Primer
gggagatcttaattaatggctagcaaaggagaagaa

SEQ ID NO: 6 - DNA, 45 bp, Artificial Sequence
  Primer
cccctcgagcggccgctgcagttatttgtagagctcatccatgcc

SEQ ID NO: 7 - DNA, 23 bp, Artificial Sequence
  Primer
gttccctggccaacacttgtcac

SEQ ID NO: 8 - DNA, 22 bp, Artificial Sequence
  Primer
tagtgacaagtgttggccaggg

SEQ ID NO: 9 - DNA, 25 bp, Artificial Sequence
  Primer
ggacacaaactggagtacaactata

SEQ ID NO: 10 - DNA, 25 bp, Artificial Sequence
  Primer
agttatagttgtactccagtttgtg

SEQ ID NO: 11 - DNA, 33 bp, Artificial Sequence
  Primer
ccgctcgagctgcagtgtaccccattaatttgg

SEQ ID NO: 12 - DNA, 35 bp, Artificial Sequence
  Primer
cgggtcgacgcggccgcgaataggacttcatacct

SEQ ID NO: 13 - DNA, 35 bp, Artificial Sequence
  Primer
cgggtcgacgcggccgcaatatgaagtcgatccta

SEQ ID NO: 14 - DNA, 35 bp, Artificial Sequence
  Primer
cgggtcgacgcggccgcgcatcccttaggggcatt

SEQ ID NO: 15 - DNA, 28 bp, Artificial Sequence
  Primer
ccgttaattaatgttcccaactattcca

SEQ ID NO: 16 - DNA, 28 bp, Artificial Sequence
  Primer
ccgttaattaatggcaactggatcaagg

SEQ ID NO: 17 - DNA, 24 bp, Artificial Sequence
  Primer
cggctcgagttaaaaaccacatga

SEQ ID NO: 18 - DNA, 29 bp, Artificial sequence
  Primer
cggctcgagttcatctttaaacctgatcc

SEQ ID NO: 19 - DNA, 593 bp, Artificial Sequence
  Human growth hormone
ttaattaaatgttcccaactattccactttctaggccattcgataacgctatgcttagggctcataggcttcatcagcttgctttcgatacttaccaagagttcgaggaggcttacattccaaaggaacagaagtactctttccttcagaacccacagacttcactttgcttctctgagtctattccaactccatctaacagggaggagactcagcagaagtctaaccttgagcttcttaggatttctcttcttcttattcagtcttggcttgagccagttcagttccttagatctgtgttcgctaactctcttgtgtacggagcttctgattctaacgtgtacgatcttcttaaggatcttgaggagggaattcagactcttatgggaaggcttgaggatggatctccaaggactggacagattttcaagcagacttactctaagttcgatacaaactctcacaacgatgatgctttgcttaagaactacggacttctttactgctttaggaaggatatggataaggtggagactttccttaggattgtgcaatgcagatctgttgagggatcttgcggattctgactcgag

SEQ ID NO: 20 - protein, 192 aa, Artificial Sequence
  Human growth hormone
MFPTIPLSRPFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 21 - DNA, 35 bp, Artificial Sequence
  B. Arthracis
tctagaaaacaatggctaaaataggataagagctc